(19) United States
(12) Patent Application Publication
(10) Pub. No.: US 2007/0105799 A1
(43) Pub. Date: May 10, 2007

(54) Codon-optimized polynucleotide-based vaccines against Bacillus anthracis infection

(75) Inventor: Gary G. Hermanson, Encinitas, CA (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Vical Incorporated

(21) Appl. No.: 10/658,688

(22) Filed: Sep. 10, 2003

Related U.S. Application Data
(60) Provisional application No. 60/419,089, filed on Oct. 18, 2002.
(60) Provisional application No. 60/409,307, filed on Sep. 10, 2002.

(51) Int. Cl. A61K 48/00; A61K 9/127; C12N 15/88; C07H 21/04

(52) U.S. Cl. 514/44; 424/450; 536/23.7; 435/458

(57) ABSTRACT

The invention is related to polynucleotide-based anthrax vaccines. In particular, the invention is plasmids operably encoding Bacillus anthracis antigens, in which the naturally-occurring coding regions for the B. anthracis antigens have been modified for improved translation in human or other mammalian cells through codon optimization. In certain embodiments, the coding regions are also modified so as to remove potential N-linked glycosylation sites. B. anthracis antigens which are useful in the invention include, but are not limited to protective antigen (PA), lethal factor (LF), and fragments, variants or derivatives of either of these antigens. The invention is further directed to methods to induce an immune response to B. anthracis in a mammal, for example, a human, comprising delivering a plasmid encoding a codon-optimized B. anthracis antigen as described above. The invention is also directed to pharmaceutical compositions comprising plasmids encoding a codon-optimized B. anthracis antigen as described above, and further comprising adjuvants, excipients, or immune modulators.

### CROSS-REFERENCE TO RELATED APPLICATIONS

The present application claims the benefit of the filing dates of U.S. Provisional Application No. 60/409,307, filed Sep. 10, 2002 and Provisional Application No. 60/419,089, filed Oct. 18, 2002, which are both incorporated herein by reference in their entireties.

### STATEMENT REGARDING FEDERALLY-SPONSORED RESEARCH

Part of the work performed during development of this invention utilized U.S. Government funds. The U.S. Government has certain rights in this invention.

### BACKGROUND OF THE INVENTION

1. Field of the Invention

Historically, anthrax infection is associated with herd animals and was not commonly seen as a human pathogen (Mock, M. and Fouet, A. Annual Review of Microbiology 55:647-671(2001)). Therefore, it is not surprising that zoonotic Bacillus anthracis infection and pathogenesis in humans is not well characterized. However, anthrax has become a greater human disease problem with the realization that anthrax spores could be weaponized. It is now widely accepted that B. anthracis spores can be inexpensively produced, are extremely stable when properly stored, and could be effectively distributed in populated areas. Consequently, B. anthracis becomes an ideal organism for use as a biological weapon and opens up the possibility of an intentional and major outbreak of infection in humans. Research during the past 10-15 years has provided an increasing amount of information about the molecular basis of disease in humans, providing the scientific basis for developing specific diagnostics and defined subunit vaccines.

2. Related Art

In addition to developing more rapid and sensitive diagnostics, molecular biological methods enable the development of defined subunit vaccines to counter bioterrorism. Indeed, safe, effective recombinant subunit vaccines would significantly reduce, and perhaps eliminate, the need for therapeutic treatments. In the case of B. anthracis, virulence is the results of a multi-component toxin secreted by the organism. The toxin consists of three separate gene products designated protective antigen (PA), lethal factor (LF) and edema factor (EF). The genes encoding these toxin components (pag, lef, and cya, respectively) are located on a 184-kb plasmid designated pXO1. pXO1, along with a second plasmid, pXO2 carrying capsule genes thought to protect bacilli from host cell phagocytosis, are required for full anthrax virulence and are carried by all virulent strains of B. anthracis (Mikesell, P., et al. Infect. Immun. 39: 371-376 (1983)). PA (735 aa, MW 82,684) is a single chain protein which binds to a mammalian cell surface receptor. Upon cleavage by furin (or a furin-like enzyme activity), it is cleaved into a 63-kDa receptor-bound product (Leppla, S. H., “Production and purification of anthrax toxin,” in Methods in Enzymology. S. Harshman, ed., Academic Press, Inc., Orlando, Fla. (1988), pp. 103-116; Klimpel, K. R., et al., Proc. Natl. Acad. Sci. (USA) 89:10277-10281 (1992); Gordon, V. M., et al., Infect. Immun. 63:82-87 (1995); Petosa, C., et al., Nature 385:8833-8838 (1997)). The 63-kDa PA fragment forms a heptameric complex on the mammalian cell surface which is capable of interacting with the 90-kDa LF protein and the 89-kDa EF protein, which are subsequently internalized (Milne, J. C., et al., J. Biol. Chem. 269:20607-20612 (1994); Petosa, C., et al., Nature 385:8833-8838 (1997)). LF (776 aa, MW 90,237) is a zinc metalloprotease that cleaves several isoforms of MAP kinase kinase (Mek1, Mek2, MKK3), thereby disrupting signal transduction events within the cell and eventually leading to cell death (Duesbery, N. S., et al., Science 280:734-737 (1998); Pellizari, R., et al., FEBS Ltrs 462:199-204. (1999)). The EF protein (767 aa, MW 88,808) is a calmodulin-dependent adenylate cyclase that causes deregulation of cellular physiology, leading to clinical manifestations that include edema (Leppla, S. H., Proc. Natl. Acad. Sci. (USA) 79:3162-3166 (1982)). The LF protein, which together with PA is referred to as lethal toxin (Letx), is considered responsible for the rapid lethality of anthrax infection (Pannifer, A., et al., Nature 414:229-232. (2001)).

Protection against anthrax infection is associated with a humoral immune response directed against PA (Ivins, B. E. and Welkos, S. L., Eur. J. Epidemiol. 4(1):12-19 (1988); Ivins, B., et al., Vaccine 13:1779-1784 (1995)). Some evidence suggests that EF and LF may also contribute to specific immunity (Little, S. F. and Knudson, G. B., Infect. Immun. 52:509-512. (1986); Ivins, B. E. and Welkos, S. L., Eur. J. Epidemiol. 4(1):12-19 (1988); Pezard, C., et al., Infect. Immun. 63:1369-1372 (1995)), although these components have not been formulated into a subunit vaccine.

The current FDA-approved anthrax vaccine, Anthrax Vaccine Adsorbed (AVA), is produced from the culture supernatant fraction of the V770-NP1-R strain of B. anthracis. Its principal component is the PA antigen adsorbed onto aluminum hydroxide. The production process is complex and the precise composition of the bacterial cell supernatant is not well characterized. Consequently, there is a significant lot-to-lot variation. In addition, the approved vaccination regimen is less than optimal for compliance and convenience: AVA is administered subcutaneously in a 0.5 ml volume, at 0, 2, and 4 weeks and then again at 6, 12, and 18 months. Annual boosts are also required.

Recently there has been a report of potential safety concerns in pregnant women, although the causal relationship has not been well established. As a result of these and other lay press reports, there is a negative public perception about the reliability and quality of the AVA vaccine even though the actual safety of the vaccine has never been seriously questioned in the scientific literature. A major concern with the current AVA anthrax vaccine is the paucity of analytical characterization of the actual composition of the vaccine preparation. It has been suggested that the presence of minute amounts of unspecified components may contribute to the adverse events that have been associated with administration of the AVA vaccine. In contrast, DNA vaccines are designed to elicit immunity against discrete, well-defined target antigens and are unlikely to be the subject of the same criticism. In short, DNA vaccines can be multivalent and yet highly defined.

During the past few years there has been substantial interest in testing DNA-based vaccines for a number of infectious diseases where the need for a vaccine, or an improved vaccine, exists. Several well-recognized advantages of DNA-based vaccines include the speed, ease and cost of manufacture, the versatility of developing and testing multivalent vaccines, the finding that DNA vaccines can produce a robust cellular response in a wide variety of animal models as well as in man, and the proven safety of using plasmid DNA as a delivery vector (Donnelly, J. J., et al., Annu. Rev. Immunol. 15:617-648 (1997); Manickan, E., et al., Crit. Rev. Immunol. 17(2):139-154 (1997)). DNA vaccines represent the next generation in the development of vaccines (Nossal, G., Nat. Med. 4(5 Supple):475-476 (1998)) and numerous DNA vaccines are in clinical trials.

DNA-based immunization have already been shown, in animal models, to protect against a lethal challenge of anthrax toxin. The initial published work indicated that a plasmid encoding the protease-cleaved fragment (PA₆₃) of PA (Gu, et al., Vaccine 17:340-344 (1999)) elicited protective immunity against a lethal toxin challenge. Price, et al., Infection and Immunity 69:4509-4515 (2001) extended these observations and demonstrated that DNA-based immunization with a fragment of the LF gene product would also contribute to or provide protection against a lethal toxin challenge. Having established proof of principle in pre-clinical studies, we now propose to develop an aggressive product development plan that will lead to an efficacious human vaccine against B. anthracis using a DNA-based immunization strategy.

Retooling coding regions encoding polypeptides from pathogens using codon frequencies preferred in a given mammalian species often results in a significant increase in expression in the cells of that mammalian species, and concomitant increase in immunogenicity. See, e.g., Deml, L., et al., J. Virol. 75:10991-11001 (2001), and Narum, D L, et al., Infect. Immun. 69:7250-7253 (2001).

There remains a need in the art for convenient, safe, and efficacious immunogenic compounds to protect vertebrates against Bacillus anthracis infection. The present invention provides safe yet effective immunogenic compounds and methods to protect vertebrates against Bacillus anthracis infection using such immunogenic compounds.

### SUMMARY OF THE INVENTION

The present invention is directed to enhancing immune response of a vertebrate in need of protection against anthrax infection by administering in vivo, into a tissue of a vertebrate, a polynucleotide comprising a codon-optimized coding region encoding a component of Bacillus anthracis lethal toxin or nucleic acid fragments of such coding regions encoding fragments, variants, or derivatives thereof. Nucleic acid fragments of the present invention are altered from their native state in one or more of the following ways. First, a nucleic acid fragment which encodes a component of the B. anthracis lethal toxin may be part or all of a codon-optimized coding region, optimized according to codon usage in a given species, e.g., a vertebrate species, e.g., a mammalian species, e.g., humans. In addition, a nucleic acid fragment which encodes a component of the B. anthracis lethal toxin may be a fragment which encodes only a portion of a full-length polypeptide, and/or may be mutated so as to, for example, remove from the encoded polypeptide adventitious protein motifs present in the encoded polypeptide or virulence factors associated with the encoded polypeptide. For example, the nucleic acid sequence could be mutated so as not to encode adventitious N-linked glycosylation motifs (N-X-(S or T), where X is any amino acid). The polynucleotides are incorporated into the cells of the vertebrate in vivo, and a prophylactically or therapeutically effective amount of a Bacillus anthracis lethal toxin component is produced in vivo.

The invention further provides immunogenic compositions comprising a polynucleotide which comprises one or more codon-optimized coding regions encoding components of Bacillus anthracis lethal toxin or nucleic acid fragments of such coding regions encoding fragments, variants, or derivatives thereof, and methods for enhancing the immune response of a vertebrate to Bacillus anthracis infection by administering to the tissues of a vertebrate one or more polynucleotides comprising one or more codon-optimized coding regions encoding components of Bacillus anthracis lethal toxin or nucleic acid fragments of such coding regions encoding fragments, variants, or derivatives thereof. The present invention further provides plasmids and other polynucleotide constructs for delivery of nucleic acid coding sequences to a vertebrate which provide expression of Bacillus anthracis toxin components, or fragments, variants, or derivatives thereof.

In certain embodiments, the invention further provides methods for enhancing the immune response of a vertebrate to Bacillus anthracis infection by sequentially administering two or more different immunogenic compositions to the tissues of the vertebrate. Such methods comprise initially administering one or more polynucleotides comprising one or more codon-optimized coding regions encoding components of Bacillus anthracis lethal toxin or nucleic acid fragments of such coding regions encoding fragments, variants, or derivatives thereof, to prime immunity, and then administering subsequently a different vaccine composition, for example a recombinant viral vaccine, a protein subunit vaccine, or a recombinant or killed bacterial vaccine or vaccines to boost the anti-Bacillus anthracis toxin immune response in the vertebrate.

### BRIEF DESCRIPTION OF THE DRAWINGS

FIG. 1 shows the nucleotide sequence (SEQ ID NO:1) and amino acid translation (SEQ ID NO:2) of TPA-PA63. SEQ ID NO:1 contains a nucleic acid fragment of a human codon-optimized PA coding region, encoding the 63 kD furin cleavage product of the Bacillus anthracis protective antigen (PA), fused to a nucleic acid encoding the human tissue plasminogen activator (TPA) signal peptide sequence. Nucleotides 1-12 of SEQ ID NO:1 is a Kozak translation initiation element and nucleotides 13-81 of SEQ ID NO:1 encode the TPA signal peptide. Nucleotides 82-1782 of SEQ ID NO:1 encode the 63 kD furin processed fragment of PA that can bind LF and EF, and heptamerize and form a pore in infected cells through which the toxin is delivered. The 63 kD furin processed fragment of PA corresponds to amino acids 199-764 of the native full-length PA amino acid sequence of GenBank accession No. AAA2263 (SEQ ID NO:4) encoded by GenBank accession number M22589 (SEQ ID NO:3).

FIG. 2 shows the nucleotide sequence (SEQ ID NO:5) and amino acid translation (SEQ ID NO:6) of TPA-PA63ΔF313-314. SEQ ID NO:5 is identical to SEQ ID NO:1, except that the nucleotides encoding the two phenylalanine residues at amino acids 313-314 of SEQ ID NO:2 are deleted, which results in a PA protein that cannot form the pore through which LF and EF are translocated. Nucleotides 1-12 of SEQ ID NO: 5 is a Kozak translation initiation element and nucleotides 13-81 of SEQ ID NO:5 encode the TPA signal peptide.

FIG. 3 shows the nucleotide sequence (SEQ ID NO:7) and amino acid translation (SEQ ID NO:8) encoding TPA-PA83 Δ Furin. SEQ ID NO:7 contains a nucleic acid fragment of a human codon-optimized PA coding region, encoding full-length mature PA (amino acids 30-764 of SEQ ID NO:4) with the furin cleavage site deleted (SRKKRS, amino acids 192-197 of SEQ ID NO:4). This mutant PA cannot be processed to the 63 kD fragment and cannot bind LF or EF. Nucleotides 1-12 of SEQ ID NO:7 is a Kozak translation initiation element and nucleotides 13-81 of SEQ ID NO:7 encode the TPA signal peptide.

FIG. 4 shows the nucleotide sequence (SEQ ID NO:9) and amino acid translation (SEQ ID NO:10) of TPA-LF HEXXH (H686A+H690A+E687D). SEQ ID NO:9 contains a nucleic acid fragment of a human codon-optimized LF coding region, encoding the mature Bacillus anthracis lethal factor with three inactivating point mutations. Either the H686A+H690A (decreased Zn binding and no protease activity) or E687D (no protease activity, no in vitro or in vivo macrophage killing) mutation inactivates the enzymatic activity of LF rendering it non-toxic (Hammond S. E. and Hanna P. C. Infect Immun. 66:2374-2378(1998)). This construct combines both sets of mutations. Nucleotides 1-12 of SEQ ID NO:9 is a Kozak translation initiation element and nucleotides 13-81 of SEQ ID NO:9 encode the TPA signal peptide. Nucleotides 82-2412 encode a non-toxic form of lethal factor. TPA-LF HEXXH (H686A+H690A+E687D) is derived from the native full-length LF amino acid sequence of GenBank accession No. AAA22569 (SEQ ID NO:12) encoded by GenBank accession number M30210 (SEQ ID NO:11).

FIG. 5 shows the nucleotide sequence (SEQ ID NO:13) and amino acid translation (SEQ ID NO:14) of TPA-LF Domain I-III. SEQ ID NO:13 contains a nucleic acid fragment of a human codon-optimized LF coding region, encoding an N-terminal fragment (domains I-III) of LF corresponding to amino acids 34-583 of SEQ ID NO:12. Nucleotides 1-12 of SEQ ID NO:13 is a Kozak translation initiation element and nucleotides 13-81 of SEQ ID NO:13 encode the TPA signal peptide. Nucleotides 82-1734 of SEQ ID NO:13 encode domains I-III of LF. The entire protease domain (domain IV) has been deleted.

FIG. 6 shows the nucleotide sequence (SEQ ID NO:15) and amino acid translation (SEQ ID NO:16) of TPA-LF Domain IA. SEQ ID NO:15 contains a nucleic acid fragment of a human codon-optimized LF coding region, encoding an LF N-terminal fragment of LF corresponding to amino acids 34-254 of SEQ ID NO:12. This truncated version of LF roughly corresponds to the domain I portion of LF that directly binds PA63. Pannifer A. D. et al. Nature 414:229-333 (2001). Nucleotides 1-12 of SEQ ID NO:15 is a Kozak translation initiation element and nucleotides 13-81 of SEQ ID NO:15 encode the TPA signal peptide. Nucleotides 82-747 of SEQ ID NO:15 encode domain I of LF.

FIG. 7 shows the nucleotide sequence (SEQ ID NO:17) and amino acid translation (SEQ ID NO:18) of TPA-PA63 with the N-linked glycosylation motifs mutated. SEQ ID NO:17 is identical to SEQ ID NO:1, except that all ten N-linked glycosylation sites have been mutated. The N residue in the glycosylation motif (N-X-S/T) has been changed to a Q residue (Q-X-S/T) resulting in a protein that cannot glycosylated at these sites. Nucleotides 1-12 of SEQ ID NO:17 is a Kozak translation initiation element and nucleotides 13-81 of SEQ ID NO:17 encode the TPA signal peptide. Nucleotides 82-747 of SEQ ID NO:15 encode domain I of LF. Nucleotides 82-1782 of SEQ ID NO:17 encode a mutated form of the 63 kD furin processed fragment of PA that can heptamerize, bind LF and EF, and form a pore in infected cells through which the toxin is delivered.

FIG. 8 shows the nucleotide sequence (SEQ ID NO:19) and amino acid translation (SEQ ID NO:20) of sugar-minus TPA-LF HEXXH mutant (H686A+H690A+E687D). SEQ ID NO:19 is identical to SEQ ID NO:9, except that all seven N-linked glycosylation sites have been mutated. The N residue in the glycosylation motif (N-X-S/T) has been changed to a Q residue (Q-X-S/T) resulting in a protein that cannot be glycosylated at these sites. Nucleotides 1-12 of SEQ ID NO:19 is a Kozak translation-initiation element and nucleotides 13-81 of SEQ ID NO:19 encode the TPA signal peptide. Nucleotides 82-2412 encode a non-toxic form of lethal factor which cannot be glycosylated.

FIG. 9 shows a nucleotide sequence comparison of a nucleic acid fragment of a human codon-optimized PA coding region, encoding PA63 (nucleotides 82-1782 of SEQ ID NO:1) vs. the native nucleotide sequence of Bacillus anthracis PA63 (nucleotides 2398-4095 of SEQ ID NO:3). Differences between the two sequences are denoted with a letter. There is approximately 25% difference in the two coding sequences.

FIG. 10 shows a nucleotide sequence comparison of a humanized nucleotide sequence encoding the mature PA Δ furin (nucleotides 82-2268 of SEQ ID NO: 7) vs. the native nucleotide sequence of Bacillus anthracis mature PA (nucleotides 1891-4095 of SEQ ID NO:3). Differences between the two sequences are denoted with a letter and gaps are denoted as a dash. There is approximately 25% difference in the two coding sequences.

FIG. 11 shows a nucleotide sequence comparison of a humanized nucleotide sequence encoding the mature LF Δ HEXXH (nucleotides 82-2409 of SEQ ID NO:9) vs. the native nucleotide sequence of Bacillus anthracis mature LF (nucleotides 784-3111 of SEQ ID NO:11). Differences between the two sequences are denoted with a letter and gaps are denoted by a gap. There is approximately 25% difference in the two coding sequences.

FIG. 12 shows an amino acid comparison between TPA-PA63 (SEQ ID NO:2) and sugar minus TPA-PA63 (SEQ ID NO:18). All ten N-linked glycosylation sites N-X-S/T in TPA-PA63 have been mutated to Q-X-S/T so that they will not be a substrate for glycosylation.

FIG. 13 shows an amino acid comparison between TPA-LFΔHEXXH (SEQ ID NO:10) and sugar minus TPA-LFΔHEXXH (SEQ ID NO:20). All seven N-linked glycosylation sites N-X-S/T in TPA-PA63 have been mutated to Q-X-S/T so that they will not be a substrate for glycosylation.

FIG. 14 shows the nucleotide sequence (SEQ ID NO:39) and amino acid translation (SEQ ID NO:40) of TPA-LF Domain IB. SEQ ID NO:39 contains a nucleic acid fragment of a human codon-optimized LF coding region, encoding an LF N-terminal fragment of LF corresponding to amino acids 34-295 of SEQ ID NO:12. This truncated version of LF roughly corresponds to the domain I portion of LF that directly binds PA63. Pannifer A. D. et al. Nature 414:229-333 (2001). Nucleotides 1-12 of SEQ ID NO:39 is a Kozak translation initiation element and nucleotides 13-81 of SEQ ID NO:39 encode the TPA signal peptide. Nucleotides 82-870 of SEQ ID NO:39 encode domain I of LF.

FIG. 15: Antibody titers measured in mouse immunization experiment 1 (Example 11). 15A: protective antigen (PA) titers; 15B: lethal factor (LF) titers; and 15C: lethal toxin (LT) neutralization titers.

FIG. 16: Antibody titers measured in mouse immunization experiment 2 (Example 11). 16A: protective antigen (PA) titers; 16B: lethal factor (LF) titers; and 16C: lethal toxin (LT) neutralization titers.

FIG. 17: Antibody titers measured in mouse immunization experiment 3 (Example 11). 17A: protective antigen (PA) titers; 17B: lethal factor (LF) titers; and 17C: lethal toxin (LT) neutralization titers.

FIG. 18: Antibody titers measured in mouse immunization experiment 4 (Example 11). 18A: protective antigen (PA) titers; 185B: lethal toxin (LT) neutralization titers.

FIG. 19: Pre-challenge lethal toxin (LT) neutralization titers in the rabbit immunization experiment (Example 12).

FIG. 20: Antibody titers measured in mouse immunization experiment 5 (Example 11).

FIG. 21: Lethal toxin (LT) neutralization titers in mouse immunization experiment 5 (Example 11).

### DETAILED DESCRIPTION OF THE INVENTION

The present invention is directed to compositions and methods for enhancing the immune response of a vertebrate in need of protection against Bacillus anthracis infection by administering in vivo, into a tissue of a vertebrate, a polynucleotide comprising a human codon-optimized coding region encoding a polypeptide of Bacillus anthracis, or a nucleic acid fragment of such a coding region encoding a fragment, variant, or derivative thereof. The polynucleotides are incorporated into the cells of the vertebrate in vivo, and an immunologically effective amount of the Bacillus anthracis polypeptide, or fragment or variant is produced in vivo.

The present invention provides polynucleotide-based vaccines and methods for delivery of Bacillus anthracis coding sequences to a vertebrate with optimal expression and safety conferred through codon optimization and/or other manipulations. These polynucleotide-based vaccines are prepared and administered in such a manner that the encoded gene products are optimally expressed in the particular vertebrate to which the composition is administered. As a result, these compositions and methods are useful in stimulating an immune response against Bacillus anthracis infection as the coding sequence encodes a polypeptide which stimulates the immune system to respond to anthrax infection. Also included in the invention are expression systems, delivery systems, and codon-optimized Bacillus anthracis coding sequences.

A polynucleotide vaccine of the present invention is capable of eliciting, without more, an immune response in a vertebrate against B. anthracis when administered to that vertebrate. Such polynucleotides are referred to herein as polynucleotide vaccines.

It is to be noted that the term “a” or “an” entity refers to one or more of that entity; for example, “a polynucleotide,” is understood to represent one or more polynucleotides. As such, the terms “a” (or “an”), “one or more,” and “at least one” can be used interchangeably herein.

The terms “nucleic acid” or “nucleic acid fragment” refers to any one or more nucleic acid segments, e.g., DNA or RNA fragments, present in a polynucleotide or construct. While the terms “nucleic acid,” as used herein, is meant to include any nucleic acid, the term “nucleic acid fragment” is used herein to specifically denote a fragment of a designed or synthetic codon-optimized coding region encoding a polypeptide, or fragment, variant, or derivative thereof, which has been optimized according to the codon usage of a given species. As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, and the like, are not part of a coding region. Two or more nucleic acids of the present invention can be present in a single polynucleotide construct, e.g., on a single plasmid, or in separate polynucleotide constructs, e.g., on separate plasmids. Furthermore, any nucleic acid or nucleic acid fragment may encode a single polypeptide, e.g., a single antigen, cytokine, or regulatory polypeptide, or may encode more than one polypeptide, e.g., a nucleic acid may encode two or more polypeptides. In addition, a nucleic acid may encode a regulatory element such as a promoter or a transcription terminator, or may encode a specialized element or motif of a polypeptide or protein, such as a secretory signal peptide or a functional domain.

The terms “fragment,” “variant,” “derivative” and “analog” when referring to B. anthracis polypeptides of the present invention include any polypeptides which retain at least some of the immunogenicity or antigenicity of the corresponding native polypeptide. Fragments of B. anthracis polypeptides of the present invention include proteolytic fragments, deletion fragments and in particular, fragments of B. anthracis polypeptides which exhibit reduced pathogenicity when delivered to an animal. Polypeptide fragments further include any portion of the polypeptide which comprises an antigenic or immunogenic epitope of the native polypeptide, including linear as well as three-dimensional epitopes. Variants of B. anthracis polypeptides of the present invention includes fragments as described above, and also polypeptides with altered amino acid sequences due to amino acid substitutions, deletions, or insertions. Variants may occur naturally, such as an allelic variant. By an “allelic variant” is intended alternate forms of a gene occupying a given locus on a chromosome of an organism. Genes II, Lewin, B., ed., John Wiley & Sons, New York (1985). Non-naturally occurring variants may be produced using art-known mutagenesis techniques. Variant polypeptides may comprise conservative or non-conservative amino acid substitutions, deletions or additions. Derivatives of B. anthracis polypeptides of the present invention, are polypeptides which have been altered so as to exhibit additional features not found on the native polypeptide. Examples include fusion proteins. An analog is another form of a B. anthracis polypeptide of the present invention, An example is a proprotein (e.g., B. anthracis PA83) which can be activated by cleavage of the proprotein to produce an active mature polypeptide (e.g., B. anthracis PA63).

The term “polynucleotide” is intended to encompass a singular nucleic acid or nucleic acid fragment as well as plural nucleic acids or nucleic acid fragments, and refers to an isolated molecule or construct, e.g., a virus genome (e.g., a non-infectious viral genome), messenger RNA (mRNA), plasmid DNA (pDNA), or derivatives of pDNA (e.g., minicircles as described in (Darquet, A-M et al., Gene Therapy 4:1341-1349 (1997)) comprising a polynucleotide. A nucleic acid may be provided in linear (e.g., mRNA), circular (e.g., plasmid), or branched form as well as double-stranded or single-stranded forms. A polynucleotide may comprise a conventional phosphodiester bond or a non-conventional bond (e.g., an amide bond, such as found in peptide nucleic acids (PNA)).

The terms “infectious polynucleotide” or “infectious nucleic acid” are intended to encompass isolated viral polynucleotides and/or nucleic acids which are solely sufficient to mediate the synthesis of complete infectious virus particles upon uptake by permissive cells. “Isolated” means that the viral nucleic acid does not require pre-synthesized copies of any of the polypeptides it encodes, e.g., viral replicases, in order to initiate its replication cycle.

The terms “non-infectious polynucleotide” or “non-infectious nucleic acid” as defined herein which cannot, without additional added materials, e.g, polypeptides, mediate the synthesis of complete infectious virus particles upon uptake by permissive cells. An infectious polynucleotide or nucleic acid is not made “non-infectious” simply because it is taken up by a non-permissive cell. For example, an infectious viral polynucleotide from a virus with limited host range is infectious if it is capable of mediating the synthesis of complete infectious virus particles when taken up by cells derived from a permissive host (i.e., a host permissive for the virus itself). The fact that uptake by cells derived from a non-permissive host does not result in the synthesis of complete infectious virus particles does not make the nucleic acid “non-infectious.” In other words, the term is not qualified by the nature of the host cell, the tissue type, or the species.

In some cases, an isolated infectious polynucleotide or nucleic acid may produce fully-infectious virus particles in a host cell population which lacks receptors for the virus particles, i.e., is non-permissive for the virus itself. Thus viruses produced will not infect surrounding cells. However, if the supernatant containing the virus particles is transferred to cells which are permissive for the virus, infection will take place.

The terms “replicating polynucleotide” or “replicating nucleic acid” are meant to encompass those polynucleotides and/or nucleic acids which, upon being taken up by a permissive host cell, are capable of producing multiple, e.g., one or more copies of the same polynucleotide or nucleic acid. Infectious polynucleotides and nucleic acids are a subset of replicating polynucleotides and nucleic acids; the terms are not synonymous. For example, a defective virus genome lacking the genes for virus coat proteins may replicate, e.g., produce multiple copies of itself, but is NOT infectious because it is incapable of mediating the synthesis of complete infectious virus particles unless the coat proteins, or another nucleic acid encoding the coat proteins, are provided.

In certain embodiments, the polynucleotide, nucleic acid, or nucleic acid fragment is DNA. In the case of DNA, a polynucleotide comprising a nucleic acid which encodes a polypeptide normally also comprises a promoter operably associated with the polypeptide-encoding nucleic acid. An operable association is when a nucleic acid encoding a gene product, e.g., a polypeptide, is associated with one or more regulatory sequences in such a way as to place expression of the gene product under the influence or control of the regulatory sequence(s). Two DNA fragments (such as a polypeptide-encoding nucleic acid and a promoter associated with the 5′ end of the nucleic acid) are “operably associated” if induction of promoter function results in the transcription of mRNA encoding the desired gene product and if the nature of the linkage between the two DNA fragments does not (1) result in the introduction of a frame-shift mutation, (2) interfere with the ability of the expression regulatory sequences to direct the expression of the gene product, or (3) interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), retroviruses (such as Rous sarcoma virus), and picornaviruses (particularly an internal ribosome entry site, or IRES, also referred to as a CITE sequence). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

In one embodiment, a DNA polynucleotide of the present invention is a circular or linearized plasmid, or other linear DNA which is, in certain embodiments, non-infectious and nonintegrating (i.e., does not integrate into the genome of vertebrate cells). A linearized plasmid is a plasmid that was previously circular but has been linearized, for example, by digestion with a restriction endonuclease.

Alternatively, DNA virus genomes may be used to administer DNA polynucleotides into vertebrate cells. In certain embodiments, a DNA virus genome of the present invention is noninfectious, and nonintegrating. Suitable DNA virus genomes include herpesvirus genomes, adenovirus genomes, adeno-associated virus genomes, and poxvirus genomes. References citing methods for the in vivo introduction of non-infectious virus genomes to vertebrate tissues are well known to those of ordinary skill in the art, and are cited supra.

In other embodiments, a polynucleotide of the present invention is RNA. In a suitable embodiment, the RNA is in the form of messenger RNA (mRNA). Methods for introducing RNA sequences into vertebrate cells are described in U.S. Pat. No. 5,580,859, the disclosure of which is incorporated herein by reference in its entirety.

Polynucleotide, nucleic acids, and nucleic acid fragments of the present invention may be associated with additional nucleic acids which encode secretory or signal peptides, which direct the secretion of a polypeptide encoded by a nucleic acid or polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native leader sequence is used, or a functional derivative of that sequence that retains the ability to direct the secretion of the polypeptide that is operably associated with it. Alternatively, a heterologous mammalian leader sequence, or a functional derivative thereof, may be used. For example, the wild-type leader sequence may be substituted with the leader sequence of human tissue plasminogen activator (TPA) or mouse β-glucuronidase.

In accordance with one aspect of the present invention, there is provided a plasmid for expression of a Bacillus anthracis PA or LF-derived coding sequence optimized for expression in the particular vertebrate species to be treated or immunized. When such a plasmid is delivered, in vivo to a tissue of the vertebrate to be treated or immunized, the transcriptional unit will thus express the encoded gene product. The level of expression of the gene product will depend to a significant extent on the strength of the associated promoter and the presence and activation of an associated enhancer element, as well as the optimization of the coding region.

As used herein, the term “plasmid” refers to a construct made up of genetic material (i.e., nucleic acids). Typically a plasmid contains an origin of replication which is functional in bacterial host cells, e.g., Escherichia coli, and selectable markers for detecting bacterial host cells comprising the plasmid. Plasmids of the present invention may include genetic elements as described herein arranged such that an inserted coding sequence can be transcribed in eukaryotic cells. Also, while the plasmid may include a sequence from a viral nucleic acid, such viral sequence normally does not cause the incorporation of the plasmid into a viral particle, and the plasmid is therefore a non-viral vector. In certain embodiments described herein, a plasmid is a closed circular DNA molecule.

The term “expression” refers to the biological production of a product encoded by a coding sequence. In most cases a DNA sequence, including the coding sequence, is transcribed to form a messenger-RNA (mRNA). The messenger-RNA is translated to form a polypeptide product which has a relevant biological activity. Also, the process of expression may involve further processing steps to the RNA product of transcription, such as splicing to remove introns, and/or post-translational processing of a polypeptide product.

As used herein, the term “polypeptide” is intended to encompass a singular “polypeptide” as well as plural “polypeptides,” and comprises any chain or chains of two or more amino acids. Thus, as used herein, terms including, but not limited to “peptide,” “dipeptide,” “tripeptide,” “protein,” “amino acid chain,” or any other term used to refer to a chain or chains of two or more amino acids, are included in the definition of a “polypeptide,” and the term “polypeptide” may be used instead of, or interchangeably with any of these terms. The term further includes polypeptides which have undergone post-translational modifications, for example, glycosylation, acetylation, phosphorylation, amidation, derivatization by known protecting/blocking groups, proteolytic cleavage, or modification by non-naturally occurring amino acids.

Also included as polypeptides of the present invention are fragments, derivatives, analogs, or variants of the foregoing polypeptides, and any combination thereof. Polypeptides, and fragments, derivatives, analogs, or variants thereof of the present invention can be antigenic and immunogenic polypeptides related to B. anthracis polypeptides, which are used to prevent or treat, i.e., cure, ameliorate, lessen the severity of, or prevent or reduce contagion of infectious disease caused by B. anthracis.

As used herein, an antigenic polypeptide or an immunogenic polypeptide is a polypeptide which, when introduced into a vertebrate, reacts with the immune system molecules of the vertebrate, i.e., is antigenic, and/or induces an immune response in the vertebrate, i.e., is immunogenic. It is quite likely that an immunogenic polypeptide will also be antigenic, but an antigenic polypeptide, because of its size or conformation, may not necessarily be immunogenic. Examples of antigenic and immunogenic polypeptides of the present invention include, but are not limited to, B. anthracis protective antigen (PA) or lethal factor (LF), fragments thereof, e.g., PA63, LF domains I-III or domain I, variants thereof, e.g., PA63Δ FF, PA83 Δ furin, PA63 sugar minus, LF HEXXH, or LF sugar minus (all described in more detail herein) and derivatives thereof, e.g., any of the foregoing polypeptides fused to a TPA signal peptide.

The term “epitopes,” as used herein, refers to portions of a polypeptide having antigenic or immunogenic activity in an animal, for example a mammal, for example, a human. An “immunogenic epitope,” as used herein, is defined as a portion of a protein that elicits an immune response in an animal, as determined by any method known in the art. The term “antigenic epitope,” as used herein, is defined as a portion of a protein to which an antibody can immunospecifically bind its antigen as determined by any method well known in the art. Immunospecific binding excludes non-specific binding but does not necessarily exclude cross-reactivity with other antigens. Antigenic epitopes need not necessarily be immunogenic.

In the present invention, antigenic epitopes preferably contain a sequence of at least 4, at least 5, at least 6, at least 7, at least 8, at least 9, at least 10, at least 15, at least 20, at least 25, or between about 15 to about 30 amino acids contained within the amino acid sequence of a polypeptide of the invention. Certain polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length. Antigenic as well as immunogenic epitopes may be linear, i.e., be comprised of contiguous amino acids in a polypeptide, or may be three dimensional, i.e., where an epitope is comprised of non-contiguous amino acids which come together due to the secondary or tertiary structure of the polypeptide, thereby forming an epitope.

The present invention is directed towards polynucleotides comprising nucleic acid fragments of codon-optimized coding regions which encode polypeptides of Bacillus anthracis, and in particular, Bacillus anthracis protective antigen (PA) or lethal factor (LF), and fragments, variants, or derivatives thereof.

“Codon optimization” is defined as modifying a nucleic acid sequence for enhanced expression in the cells of the vertebrate of interest by replacing at least one, more than one, or a significant number, of codons of the native sequence with codons that are more frequently or most frequently used in the genes of that vertebrate. Various species exhibit particular bias for certain codons of a particular amino acid.

The present invention relates to polynucleotides comprising nucleic acid fragments of codon-optimized coding regions which encode Bacillus anthracis polypeptides, with the codon usage adapted for optimized expression in the cells of a given vertebrate. These polynucleotides are prepared by incorporating codons preferred for use in the genes of a given species into the DNA sequence. Also provided are polynucleotide expression constructs, vectors, host cells comprising nucleic acid fragments of codon-optimized coding regions which encode Bacillus anthracis polypeptides, and various methods of using the polynucleotide expression constructs, vectors, host cells to treat or prevent anthrax in a vertebrate.

Codon Optimization

As used herein the term “codon optimized coding region” means a nucleic acid coding region that has been adapted for expression in the cells of a given vertebrate by replacing at least one, or more than one, or a significant number, of codons with one or more codons that are more frequently used in the genes of that vertebrate.

Deviations in the nucleotide sequence that comprise the codons encoding the amino acids of any polypeptide chain allow for variations in the sequence coding for the gene. Since each codon consists of three nucleotides, and the nucleotides comprising DNA are restricted to four specific bases, there are 64 possible combinations of nucleotides, 61 of which encode amino acids (the remaining three codons encode signals ending translation). The “genetic code” which shows which codons encode which amino acids is reproduced herein as Table 1. As a result, many amino acids are designated by more than one codon. For example, the amino acids alanine and proline are coded for by four triplets, serine and arginine by six, whereas tryptophan and methionine are coded by just one triplet. This degeneracy allows for DNA base composition to vary over a wide range without altering the amino acid sequence of the proteins encoded by the DNA.

TABLE 1
The Standard Genetic Code

| | | | |
|---|---|---|---|
| T | C | A | G |

| | | | | |
|---|---|---|---|---|
| T | TTT Phe (F) | TCT Ser (S) | TAT Tyr (Y) | TGT Cys (C) |
| | TTC″ | TCC″ | TAC″ | TGC |
| | TTA Leu(L) | TCA″ | TAA Ter | TGA Ter |
| TTG″ | TCG″ | TAG Ter | TGG Trp(W) | |
| C | CTT Leu (L) | CCT Pro (P) | CAT His (H) | CGT Arg (R) |
| | CTC″ | CCC″ | CAC″ | CGC″ |
| | CTA″ | CCA″ | CAA Gln (Q) | CGA″ |
| | CTG″ | CCG″ | CAG″ | CGG″ |
| A | ATT Ile (I) | ACT Thr (T) | AAT Asn (N) | AGT Ser (S) |
| | ATC | ACC″ | AAC″ | AGC″ |
| | ATA | ACA″ | AAA Lys (K) | AGA Arg (R) |
| | ATG Met (M) | ACG″ | AAG″ | AGG″ |
| G | GTT Val (V) | GCT Ala (A) | GAT Asp (D) | GGT Gly (G) |
| | GTC″ | GCC″ | GAC″ | GGC″ |
| | GTA″ | GCA″ | GAA Glu (E) | GGA″ |
| | GTG″ | GCG″ | GAG″ | GGG″ |

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

Given the large number of gene sequences available for a wide variety of animal, plant and microbial species, it is possible to calculate the relative frequencies of codon usage. Codon usage tables are readily available, for example, at the “Codon Usage Database” available at http://www.kazusa.or.jp/codon/ (visited Jul. 9, 2002), and these tables can be adapted in a number of ways. See Nakamura, Y., et al. “Codon usage tabulated from the international DNA sequence databases: status for the year 2000” Nucl. Acids Res. 28:292 (2000). Codon usage tables for human, mouse, domestic cat, and cow, calculated from GenBank Release 128.0 [15 February 2002], are reproduced below as Tables 2-5. These tables use mRNA nomenclature, and so instead of thymine (T) which is found in DNA, the tables use uracil (U) which is found in RNA. The tables have been adapted so that frequencies are calculated for each amino acid, rather than for all 64 codons.

TABLE 2
Codon Usage Table for Human Genes (Homo sapiens)

| Amino Acid | Codon | Number | Frequency |
|---|---|---|---|
| Phe | UUU | 326146 | 0.4525 |
| Phe | UUC | 394680 | 0.5475 |
| Total | | 720826 | |
| Leu | UUA | 139249 | 0.0728 |
| Leu | UUG | 242151 | 0.1266 |
| Leu | CUU | 246206 | 0.1287 |
| Leu | CUC | 374262 | 0.1956 |
| Leu | CUA | 133980 | 0.0700 |
| Leu | CUG | 777077 | 0.4062 |
| Total | | 1912925 | |
| Ile | AUU | 303721 | 0.3554 |
| Ile | AUC | 414483 | 0.4850 |
| Ile | AUA | 136399 | 0.1596 |
| Total | | 854603 | |
| Met | AUG | 430946 | 1.0000 |
| Total | | 430946 | |
| Val | GUU | 210423 | 0.1773 |
| Val | GUC | 282445 | 0.2380 |
| Val | GUA | 134991 | 0.1137 |
| Val | GUG | 559044 | 0.4710 |
| Total | | 1186903 | |
| Ser | UCU | 282407 | 0.1840 |
| Ser | UCC | 336349 | 0.2191 |
| Ser | UCA | 225963 | 0.1472 |
| Ser | UCG | 86761 | 0.0565 |
| Ser | AGU | 230047 | 0.1499 |
| Ser | AGC | 373362 | 0.2433 |
| Total | | 1534889 | |
| Pro | CCU | 333705 | 0.2834 |
| Pro | CCC | 386462 | 0.3281 |
| Pro | CCA | 322220 | 0.2736 |
| Pro | CCG | 135317 | 0.1149 |
| Total | | 1177704 | |
| Thr | ACU | 247913 | 0.2419 |
| Thr | ACC | 371420 | 0.3624 |
| Thr | ACA | 285655 | 0.2787 |
| Thr | ACG | 120022 | 0.1171 |
| Total | | 1025010 | |
| Ala | GCU | 360146 | 0.2637 |
| Ala | GCC | 551452 | 0.4037 |
| Ala | GCA | 308034 | 0.2255 |
| Ala | GCG | 146233 | 0.1071 |
| Total | | 1365865 | |
| Tyr | UAU | 232240 | 0.4347 |
| Tyr | UAC | 301978 | 0.5653 |
| Total | | 534218 | |
| His | CAU | 201389 | 0.4113 |
| His | CAC | 288200 | 0.5887 |
| Total | | 489589 | |
| Gln | CAA | 227742 | 0.2541 |
| Gln | CAG | 668391 | 0.7459 |
| Total | | 896133 | |
| Asn | AAU | 322271 | 0.4614 |
| Asn | AAC | 376210 | 0.5386 |
| Total | | 698481 | |
| Lys | AAA | 462660 | 0.4212 |
| Lys | AAG | 635755 | 0.5788 |
| Total | | 1098415 | |
| Asp | GAU | 430744 | 0.4613 |
| Asp | GAC | 502940 | 0.5387 |
| Total | | 933684 | |
| Glu | GAA | 561277 | 0.4161 |
| Glu | GAG | 787712 | 0.5839 |
| Total | | 1348989 | |
| Cys | UGU | 190962 | 0.4468 |
| Cys | UGC | 236400 | 0.5532 |
| Total | | 427362 | |
| Trp | UGG | 248083 | 1.0000 |
| Total | | 248083 | |
| Arg | CGU | 90899 | 0.0830 |
| Arg | CGC | 210931 | 0.1927 |
| Arg | CGA | 122555 | 0.1120 |
| Arg | CGG | 228970 | 0.2092 |
| Arg | AGA | 221221 | 0.2021 |
| Arg | AGG | 220119 | 0.2011 |
| Total | | 1094695 | |
| Gly | GGU | 209450 | 0.1632 |
| Gly | GGC | 441320 | 0.3438 |
| Gly | GGA | 315726 | 0.2459 |
| Gly | GGG | 317263 | 0.2471 |
| Total | | 1283759 | |
| Stop | UAA | 13963 | |
| Stop | UAG | 10631 | |
| Stop | UGA | 24607 | |

TABLE 3
Codon Usage Table for Mouse Genes (Mus musculus)

| Amino Acid | Codon | Number | Frequency |
|---|---|---|---|
| Phe | UUU | 150467 | 0.4321 |
| Phe | UUC | 197795 | 0.5679 |
| Total | | 348262 | |
| Leu | UUA | 55635 | 0.0625 |
| Leu | UUG | 116210 | 0.1306 |
| Leu | CUU | 114699 | 0.1289 |
| Leu | CUC | 179248 | 0.2015 |
| Leu | CUA | 69237 | 0.0778 |
| Leu | CUG | 354743 | 0.3987 |
| Total | | 889772 | |
| Ile | AUU | 137513 | 0.3367 |
| Ile | AUC | 208533 | 0.5106 |
| Ile | AUA | 62349 | 0.1527 |
| Total | | 408395 | |
| Met | AUG | 204546 | 1.0000 |
| Total | | 204546 | |
| Val | GUU | 93754 | 0.1673 |
| Val | GUC | 140762 | 0.2513 |
| Val | GUA | 64417 | 0.1150 |
| Val | GUG | 261308 | 0.4664 |
| Total | | 560241 | |
| Ser | UCU | 139576 | 0.1936 |
| Ser | UCC | 160313 | 0.2224 |
| Ser | UCA | 100524 | 0.1394 |
| Ser | UCG | 38632 | 0.0536 |
| Ser | AGU | 108413 | 0.1504 |
| Ser | AGC | 173518 | 0.2407 |
| Total | | 720976 | |
| Pro | CCU | 162613 | 0.3036 |
| Pro | CCC | 164796 | 0.3077 |
| Pro | CCA | 151091 | 0.2821 |
| Pro | CCG | 57032 | 0.1065 |
| Total | | 535532 | |
| Thr | ACU | 119832 | 0.2472 |
| Thr | ACC | 172415 | 0.3556 |
| Thr | ACA | 140420 | 0.2896 |
| Thr | ACG | 52142 | 0.1076 |
| Total | | 484809 | |
| Ala | GCU | 178593 | 0.2905 |
| Ala | GCC | 236018 | 0.3839 |
| Ala | GCA | 139697 | 0.2272 |
| Ala | GCG | 60444 | 0.0983 |
| Total | | 614752 | |
| Tyr | UAU | 108556 | 0.4219 |
| Tyr | UAC | 148772 | 0.5781 |
| Total | | 257328 | |
| His | CAU | 88786 | 0.3973 |
| His | CAC | 134705 | 0.6027 |
| Total | | 223491 | |
| Gln | CAA | 101783 | 0.2520 |
| Gln | CAG | 302064 | 0.7480 |
| Total | | 403847 | |
| Asn | AAU | 138868 | 0.4254 |
| Asn | AAC | 187541 | 0.5746 |
| Total | | 326409 | |
| Lys | AAA | 188707 | 0.3839 |
| Lys | AAG | 302799 | 0.6161 |
| Total | | 491506 | |
| Asp | GAU | 189372 | 0.4414 |
| Asp | GAC | 239670 | 0.5586 |
| Total | | 429042 | |
| Glu | GAA | 235842 | 0.4015 |
| Glu | GAG | 351582 | 0.5985 |
| Total | | 587424 | |
| Cys | UGU | 97385 | 0.4716 |
| Cys | UGC | 109130 | 0.5284 |
| Total | | 206515 | |
| Trp | UGG | 112588 | 1.0000 |
| Total | | 112588 | |
| Arg | CGU | 41703 | 0.0863 |
| Arg | CGC | 86351 | 0.1787 |
| Arg | CGA | 58928 | 0.1220 |
| Arg | CGG | 92277 | 0.1910 |
| Arg | AGA | 101029 | 0.2091 |
| Arg | AGG | 102859 | 0.2129 |
| Total | | 483147 | |
| Gly | GGU | 103673 | 0.1750 |
| Gly | GGC | 198604 | 0.3352 |
| Gly | GGA | 151497 | 0.2557 |
| Gly | GGG | 138700 | 0.2341 |
| Total | | 592474 | |
| Stop | UAA | 5499 | |
| Stop | UAG | 4661 | |
| Stop | UGA | 10356 | |

TABLE 4
Codon Usage Table for Domestic Cat Genes (Felis cattus)

| Amino Acid | Codon | Number | Frequency of usage |
|---|---|---|---|
| Phe | UUU | 1204.00 | 0.4039 |
| Phe | UUC | 1777.00 | 0.5961 |
| Total | | 2981 | |
| Leu | UUA | 404.00 | 0.0570 |
| Leu | UUG | 857.00 | 0.1209 |
| Leu | CUU | 791.00 | 0.1116 |
| Leu | CUC | 1513.00 | 0.2135 |
| Leu | CUA | 488.00 | 0.0688 |
| Leu | CUG | 3035.00 | 0.4282 |
| Total | | 7088 | |
| Ile | AUU | 1018.00 | 0.2984 |
| Ile | AUC | 1835.00 | 0.5380 |
| Ile | AUA | 558.00 | 0.1636 |
| Total | | 3411 | |
| Met | AUG | 1553.00 | 0.0036 |
| Total | | 1553 | |
| Val | GUU | 696.00 | 0.1512 |
| Val | GUC | 1279.00 | 0.2779 |
| Val | GUA | 463.00 | 0.1006 |
| Val | GUG | 2164.00 | 0.4702 |
| Total | | 4602 | |
| Ser | UCU | 940.00 | 0.1875 |
| Ser | UCC | 1260.00 | 0.2513 |
| Ser | UCA | 608.00 | 0.1213 |
| Ser | UCG | 332.00 | 0.0662 |
| Ser | AGU | 672.00 | 0.1340 |
| Ser | AGC | 1202.00 | 0.2397 |
| Total | | 5014 | |
| Pro | CCU | 958.00 | 0.2626 |
| Pro | CCC | 1375.00 | 0.3769 |
| Pro | CCA | 850.00 | 0.2330 |
| Pro | CCG | 465.00 | 0.1275 |
| Total | | 3648 | |
| Thr | ACU | 822.00 | 0.2127 |
| Thr | ACC | 1574.00 | 0.4072 |
| Thr | ACA | 903.00 | 0.2336 |
| Thr | ACG | 566.00 | 0.1464 |
| Total | | 3865 | |
| Ala | GCU | 1129.00 | 0.2496 |
| Ala | GCC | 1951.00 | 0.4313 |
| Ala | GCA | 883.00 | 0.1952 |
| Ala | GCG | 561.00 | 0.1240 |
| Total | | 4524 | |
| Tyr | UAU | 837.00 | 0.3779 |
| Tyr | UAC | 1378.00 | 0.6221 |
| Total | | 2215 | |
| His | CAU | 594.00 | 0.3738 |
| His | CAC | 995.00 | 0.6262 |
| Total | | 1589 | |
| Gln | CAA | 747.00 | 0.2783 |
| Gln | CAG | 1937.00 | 0.7217 |
| Total | | 2684 | |
| Asn | AAU | 1109.00 | 0.3949 |
| Asn | AAC | 1699.00 | 0.6051 |
| Total | | 2808 | |
| Lys | AAA | 1445.00 | 0.4088 |
| Lys | AAG | 2090.00 | 0.5912 |
| Total | | 3535 | |
| Asp | GAU | 1255.00 | 0.4055 |
| Asp | GAC | 1840.00 | 0.5945 |
| Total | | 3095 | |
| Glu | GAA | 1637.00 | 0.4164 |
| Glu | GAG | 2294.00 | 0.5836 |
| Total | | 3931 | |
| Cys | UGU | 719.00 | 0.4425 |
| Cys | UGC | 906.00 | 0.5575 |
| Total | | 1625 | |
| Trp | UGG | 1073.00 | 1.0000 |
| Total | | 1073 | |
| Arg | CGU | 236.00 | 0.0700 |
| Arg | CGC | 629.00 | 0.1865 |
| Arg | CGA | 354.00 | 0.1050 |
| Arg | CGG | 662.00 | 0.1963 |
| Arg | AGA | 712.00 | 0.2112 |
| Arg | AGG | 779.00 | 0.2310 |
| Total | | 3372 | |
| Gly | GGU | 648.00 | 0.1498 |
| Gly | GGC | 1536.00 | 0.3551 |
| Gly | GGA | 1065.00 | 0.2462 |
| Gly | GGG | 1077.00 | 0.2490 |
| Total | | 4326 | |
| Stop | UAA | 55 | |
| Stop | UAG | 36 | |
| Stop | UGA | 110 | |

TABLE 5
Codon Usage Table for Cow Genes (Bos taurus)

| Amino Acid | Codon | Number | Frequency of usage |
|---|---|---|---|
| Phe | UUU | 13002 | 0.4112 |
| Phe | UUC | 18614 | 0.5888 |
| Total | | 31616 | |
| Leu | UUA | 4467 | 0.0590 |
| Leu | UUG | 9024 | 0.1192 |
| Leu | CUU | 9069 | 0.1198 |
| Leu | CUC | 16003 | 0.2114 |
| Leu | CUA | 4608 | 0.0609 |
| Leu | CUG | 32536 | 0.4298 |
| Total | | 75707 | |
| Ile | AUU | 12474 | 0.3313 |
| Ile | AUC | 19800 | 0.5258 |
| Ile | AUA | 5381 | 0.1429 |
| Total | | 37655 | |
| Met | AUG | 17770 | 1.0000 |
| Total | | 17770 | |
| Val | GUU | 8212 | 0.1635 |
| Val | GUC | 12846 | 0.2558 |
| Val | GUA | 4932 | 0.0982 |
| Val | GUG | 24222 | 0.4824 |
| Total | | 50212 | |
| Ser | UCU | 10287 | 0.1804 |
| Ser | UCC | 13258 | 0.2325 |
| Ser | UCA | 7678 | 0.1347 |
| Ser | UCG | 3470 | 0.0609 |
| Ser | AGU | 8040 | 0.1410 |
| Ser | AGC | 14279 | 0.2505 |
| Total | | 57012 | |
| Pro | CCU | 11695 | 0.2684 |
| Pro | CCC | 15221 | 0.3493 |
| Pro | CCA | 11039 | 0.2533 |
| Pro | CCG | 5621 | 0.1290 |
| Total | | 43576 | |
| Thr | ACU | 9372 | 0.2203 |
| Thr | ACC | 16574 | 0.3895 |
| Thr | ACA | 10892 | 0.2560 |
| Thr | ACG | 5712 | 0.1342 |
| Total | | 42550 | |
| Ala | GCU | 13923 | 0.2592 |
| Ala | GCC | 23073 | 0.4295 |
| Ala | GCA | 10704 | 0.1992 |
| Ala | GCG | 6025 | 0.1121 |
| Total | | 53725 | |
| Tyr | UAU | 9441 | 0.3882 |
| Tyr | UAC | 14882 | 0.6118 |
| Total | | 24323 | |
| His | CAU | 6528 | 0.3649 |
| His | CAC | 11363 | 0.6351 |
| Total | | 17891 | |
| Gln | CAA | 8060 | 0.2430 |
| Gln | CAG | 25108 | 0.7570 |
| Total | | 33168 | |
| Asn | AAU | 12491 | 0.4088 |
| Asn | AAC | 18063 | 0.5912 |
| Total | | 30554 | |
| Lys | AAA | 17244 | 0.3897 |
| Lys | AAG | 27000 | 0.6103 |
| Total | | 44244 | |
| Asp | GAU | 16615 | 0.4239 |
| Asp | GAC | 22580 | 0.5761 |
| Total | | 39195 | |
| Glu | GAA | 21102 | 0.4007 |
| Glu | GAG | 31555 | 0.5993 |
| Total | | 52657 | |
| Cys | UGU | 7556 | 0.4200 |
| Cys | UGC | 10436 | 0.5800 |
| Total | | 17992 | |
| Trp | UGG | 10706 | 1.0000 |
| Total | | 10706 | |
| Arg | CGU | 3391 | 0.0824 |
| Arg | CGC | 7998 | 0.1943 |
| Arg | CGA | 4558 | 0.1108 |
| Arg | CGG | 8300 | 0.2017 |
| Arg | AGA | 8237 | 0.2001 |
| Arg | AGG | 8671 | 0.2107 |
| Total | | 41155 | |
| Gly | GGU | 8508 | 0.1616 |
| Gly | GGC | 18517 | 0.3518 |
| Gly | GGA | 12838 | 0.2439 |
| Gly | GGG | 12772 | 0.2427 |
| Total | | 52635 | |
| Stop | UAA | 555 | |
| Stop | UAG | 394 | |
| Stop | UGA | 392 | |

By utilizing these or similar tables, one of ordinary skill in the art can apply the frequencies to any given polypeptide sequence, and produce a nucleic acid fragment of a codon-optimized coding region which encodes the polypeptide, but which uses codons optimal for a given species. Codon-optimized coding regions can be designed by various different methods.

In one method, a codon usage table is used to find the single most frequent codon used for any given amino acid, and that codon is used each time that particular amino acid appears in the polypeptide sequence. For example, referring to Table 2 above, for leucine, the most frequent codon is CUG, which is used 41% of the time. Thus all the leucine residues in a given amino acid sequence would be assigned the codon CUG. Human codon-optimized nucleotide sequences encoding native PA (GenBank Accession Number AAA2263 (SEQ ID NO:4)) and native LF (GenBank Accession Number AAA22569 (SEQ ID NO:12)) which have been optimized using this method are presented herein as SEQ ID NO:21 and SEQ ID NO:22, respectively.

In another method, the actual frequencies of the codons are distributed randomly throughout the coding sequence. Thus using this method for optimization, if a hypothetical polypeptide sequence had 100 leucine residues, referring to Table 2 for frequency of usage in the humans, about 7, or 7% of the leucine codons would be UUA, about 13, or 13% of the leucine codons would be UUG, about 13, or 13% of the leucine codons would be CUU, about 20, or 20% of the leucine codons would be CUC, about 7, or 7% of the leucine codons would be CUA, and about 41, or 41% of the leucine codons would be CUG. These frequencies would be distributed randomly throughout the leucine codons in the coding region encoding the hypothetical polypeptide. As will be understood by those of ordinary skill in the art, the distribution of codons in the sequence will can vary significantly using this method, however, the sequence always encodes the same polypeptide. Three different human codon-optimized nucleotide sequences encoding native PA (GenBank Accession Number AAA2263 (SEQ ID NO:4)) which have been optimized using this method are presented herein as SEQ ID NO:23, SEQ ID NO:24, and SEQ ID NO:25. Three different human codon-optimized sequences encoding native LF (GenBank Accession Number AAA22569 (SEQ ID NO:12)) which have been optimized using this method are presented herein as SEQ ID NO:21 and SEQ ID NO:22, respectively.

When using the latter method, the term “about” is used precisely to account for fractional percentages of codon frequencies for a given amino acid. As used herein, “about” is defined as one amino acid more or one amino acid less than the value given. The whole number value of amino acids is rounded up if the fractional frequency of usage is 0.50 or greater, and is rounded down if the fractional frequency of use is 0.49 or less. Using again the example of the frequency of usage of leucine in human genes for a hypothetical polypeptide having 62 leucine residues, the fractional frequency of codon usage would be calculated by multiplying 62 by the frequencies for the various codons. Thus, 7.28 percent of 62 equals 4.51 UUA codons, or “about 5,” i.e., 4, 5, or 6 UUA codons, 12.66 percent of 62 equals 7.85 UUG codons or “about 8,” i.e., 7, 8, or 9 UUG codons, 12.87 percent of 62 equals 7.98 CUU codons, or “about 8,” i.e., 7, 8, or 9 CUU codons, 19.56 percent of 62 equals 12.13 CUC codons or “about 12,” i.e., 11, 12, or 13 CUC codons, 7.00 percent of 62 equals 4.34 CUA codons or “about 4,” i.e., 3, 4, or 5 CUA codons, and 40.62 percent of 62 equals 25.19 CUG codons, or “about 25,” i.e., 24, 25, or 26 CUG codons.

Randomly assigning codons at an optimized frequency to encode a given polypeptide sequence, can be done manually by calculating codon frequencies for each amino acid, and then assigning the codons to the polypeptide sequence randomly. Additionally, various algorithms and computer software programs are readily available to those of ordinary skill in the art. For example, the “EditSeq” function in the Lasergene Package, available from DNAstar, Inc., Madison, Wis., the backtranslation function in the Vector NTI Suite, available from InforMax, Inc., Bethesda, Md., and the “backtranslate” function in the GCG-Wisconsin Package, available from Accelrys, Inc., San Diego, Calif. In addition, various resources are publicly available to codon-optimize coding region sequences. For example, the “backtranslation” function at http://www.entelechon.com/eng/backtranslation.html (visited Jul. 9, 2002), the “backtranseq” function available at http://bioinfo.pbi.nrc.ca:8090/EMBOSS/index.html (visited Jul. 9, 2002). Constructing a rudimentary algorithm to assign codons based on a given frequency can also easily be accomplished with basic mathematical functions by one of ordinary skill.

A number of options are available for synthesizing codon optimized coding regions designed by any of the methods described above, using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides is designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

In certain embodiments, an entire polypeptide sequence, or fragment, variant, or derivative thereof is codon optimized by any of the methods described herein. Various desired fragments, variants or derivatives are designed, and each is then codon-optimized individually. In addition, partially codon-optimized coding regions of the present invention can be designed and constructed. For example, the invention includes a nucleic acid fragment of a codon-optimized coding region encoding a polypeptide in which at least about 1%, 2%, 3,% 4%, 5%, 10%, 15%, 20%, 25%, 30%, 35%, 40%, 45%, 50%, 55%, 60%, 65%, 70%, 75%, 80%, 85%, 90%, 95%, or 100% of the codon positions have been codon-optimized for a given species. That is, they contain a codon that is preferentially used in the genes of a desired species, e.g., a vertebrate species, e.g., humans, in place of a codon that is normally used in the native nucleic acid sequence.

In additional embodiments, a full-length polypeptide sequence is codon-optimized for a given species resulting in a codon-optimized coding region encoding the entire polypeptide, and then nucleic acid fragments of the codon-optimized coding region, which encode fragments, variants, and derivatives of the polypeptide are made from the original codon-optimized coding region. As would be well understood by those of ordinary skill in the art, if codons have been randomly assigned to the full-length coding region based on their frequency of use in a given species, nucleic acid fragments encoding fragments, variants, and derivatives would not necessarily be fully codon optimized for the given species. However, such sequences are still much closer to the codon usage of the desired species than the native codon usage. The advantage of this approach is that synthesizing codon-optimized nucleic acid fragments encoding each fragment, variant, and derivative of a given polypeptide, although routine, would be time consuming and would result in significant expense.

The codon-optimized coding regions can be versions encoding any gene products from any strain of Bacillus anthracis, or fragments, variants, or derivatives of such gene products. Described herein are nucleic acid fragments of codon-optimized coding regions encoding the Bacillus anthracis protective antigen (PA) gene and the Bacillus anthracis lethal factor (LF), the nucleic acid fragments encoding the complete polypeptide, as well as various fragments, variants, and derivatives thereof, although other PA or LF-encoding nucleic acid sources are not excluded.

The present invention is directed to compositions and methods of enhancing the immune response of a vertebrate in need of protection against Bacillus anthracis infection by administering in vivo, into a tissue of a vertebrate, a polynucleotide comprising a codon-optimized coding region encoding a polypeptide of Bacillus anthracis, or a nucleic acid fragment of such a coding region encoding a fragment, variant or derivative thereof. Codon optimization is carried out for a particular vertebrate species by methods described herein, for example, in certain embodiments codon-optimized coding regions encoding polypeptides of Bacillus anthracis, or nucleic acid fragments of such coding regions encoding fragments, variants, or derivatives thereof are optimized according to human codon usage. The polynucleotides of the invention are incorporated into the cells of the vertebrate in vivo, and an immunologically effective amount of a Bacillus anthracis polypeptide is produced in vivo. In particular, the present invention relates to codon-optimized coding regions encoding polypeptides of Bacillus anthracis, or nucleic acid fragments of such coding regions fragments, variants, or derivatives thereof which have been optimized according to mammalian codon usage, for example, human codon usage, cow codon usage, domestic cat codon usage, or mouse codon usage. For example, human codon-optimized coding regions encoding polypeptides of Bacillus anthracis, or nucleic acid fragments of such coding regions encoding fragments, variants, or derivatives thereof are prepared by incorporating codons preferred for use in human genes into the DNA sequence encoding the B. anthracis polypeptide. Also provided are polynucleotides, vectors, and other expression constructs comprising codon-optimized coding regions encoding polypeptides of Bacillus anthracis, or nucleic acid fragments of such coding regions encoding fragments, variants, or derivatives thereof, and various methods of using such polynucleotides, vectors and other expression constructs.

The present invention is further directed towards polynucleotides comprising codon-optimized coding regions encoding polypeptides of Bacillus anthracis toxin, for example, Bacillus anthracis lethal toxin and its component polypeptides, for example, lethal factor (LF) and protective antigen (PA). The invention is also directed to polynucleotides comprising codon-optimized nucleic acid fragments encoding fragments, variants and derivatives of these polypeptides.

The present invention provides isolated polynucleotides comprising codon-optimized coding regions of Bacillus anthracis PA, or fragments, variants, or derivatives thereof. In certain embodiments described herein, a codon-optimized coding region encoding SEQ ID NO:4 is optimized according to codon usage in humans (Homo sapiens). Alternatively, a codon-optimized coding region encoding SEQ ID NO:4 may be optimized according to codon usage in any plant, animal, or microbial species.

Codon-optimized coding regions encoding SEQ ID NO:4, optimized according to codon usage in humans are designed as follows. The amino acid composition of SEQ ID NO:4 is shown in Table 6.

TABLE 6

| | |
|---|---|
| Amino | Number in |
| Acid | SEQ ID NO: 4 |

| | | |
|---|---|---|
| A | Ala | 41 |
| R | Arg | 29 |
| C | Cys | 0 |
| G | Gly | 36 |
| H | His | 10 |
| I | Ile | 57 |
| L | Leu | 62 |
| K | Lys | 60 |
| M | Met | 10 |
| F | Phe | 24 |
| P | Pro | 29 |
| S | Ser | 72 |
| T | Thr | 58 |
| W | Trp | 7 |
| Y | Tyr | 28 |
| V | Val | 43 |
| N | Asn | 69 |
| D | Asp | 47 |
| Q | Gln | 31 |
| E | Glu | 51 |

Using the amino acid composition shown in Table 6, a human codon-optimized coding region which encodes SEQ ID NO:4 can be designed by any of the methods discussed herein. In the first approach, each amino acid is assigned the most frequent codon used in the human genome for that amino acid. According to this method, codons are assigned to the coding region encoding SEQ ID NO:4 as follows: the 24 phenylalanine codons are TTC, the 62 leucine codons are CTG, the 57 isoleucine codons are ATC, the 10 methionine codons are ATG, the 43 valine codons are GTG, the 72 serine codons are AGC, the 29 proline codons are CCC, the 58 threonine codons are ACC, the 41 alanine codons are GCC, the 28 tyrosine codons are TAC, the 10 histidine codons are CAC, the 31 glutamine codons are CAG, the 69 asparagine codons are AAC, the 60 lysine codons are AAG, the 47 aspartic acid codons are GAC, the 51 glutamic acid codons are GAG, the 7 tryptophan codons are TGG, the 29 arginine codons are CGG, AGA, or AGG (the frequencies of usage of these three codons in the human genome are not significantly different), and the 36 glycine codons are GGC. The codon-optimized PA coding region designed by this method is presented herein as SEQ ID NO:21.

Alternatively, a human codon-optimized coding region which encodes SEQ ID NO:4 can be designed by randomly assigning each of any given amino acid a codon based on the frequency that codon is used in the human genome. These frequencies are shown in Table 2 above. Using this latter method, codons are assigned to the coding region encoding SEQ ID NO:4 as follows: about 11 of the 24 phenylalanine codons are TTT, and about 13 of the phenylalanine codons are TTC; about 5 of the 62 leucine codons are TTA, about 8 of the leucine codons are TTG, about 8 of the leucine codons are CTT, about 12 of the leucine codons are CTC, about 4 of the leucine codons are CTA, and about 25 of the leucine codons are CTG; about 20 of the 57 isoleucine codons are ATT, about 28 of the isoleucine codons are ATC, and about 9 of the isoleucine codons are ATA; the 10 methionine codons are ATG; about 8 of the 43 valine codons are GTT, about 10 of the valine codons are GTG, about 5 of the valine codons are GTA, and about 20 of the valine codons are GTG; about 13 of the 72 serine codons are TCT, about 16 of the serine codons are TCC, about 11 of the serine codons are TCA, about 4 of the serine codons are TCG, about 11 of the serine codons are AGT, and about 17 of the serine codons are AGC; about 8 of the 29 proline codons are CCT, about 10 of the proline codons are CCC, about 8 of the proline codons are CCA, and about 3 of the proline codons are CCG; about 14 of the 58 threonine codons are ACT, about 21 of the threonine codons are ACC, about 16 of the threonine codons are ACA, and about 7 of the threonine codons are ACG; about 11 of the 41 alanine codons are GGT, about 17 of the alanine codons are GCC, about 9 of the alanine codons are GCA, and about 4 of the alanine codons are GCG; about 12 of the 28 tyrosine codons are TAT and about 16 of the tyrosine codons are TAC; about 4 of the 10 histidine codons are CAT and about 6 of the histidine codons are CAC; about 8 of the 31 glutamine codons are CAA and about 23 of the glutamine codons are CAG; about 32 of the 69 asparagine codons are AAT and about 37 of the asparagine codons are AAC; about 25 of the 60 lysine codons are AAA and about 35 of the lysine codons are AAG; about 22 of the 47 aspartic acid codons are GAT and about 25 of the aspartic acid codons are GAC; about 21 of the 51 glutamic acid codons are GAA and about 30 of the glutamic acid codons are GAG; the 7 tryptophan codons are TGG; about 2 of the 29 arginine codons are CGT, about 6 of the arginine codons are CGC, about 3 of the arginine codons are CGA, about 6 of the arginine codons are CGG, about 6 of the arginine codons are AGA, and about 6 of the arginine codons are AGG; and about 6 of the 36 glycine codons are GGT, about 12 of the glycine codons are GGC, about 9 of the glycine codons are GGA, and about 9 of the glycine codons are GGG.

As described above, the term “about” means that the number of amino acids encoded by a certain codon may be one more or one less than the number given. It would be understood by those of ordinary skill in the art that the total number of any amino acid in the polypeptide sequence must remain constant, therefore, if there is one “more” of one codon encoding a give amino acid, there would have to be one “less” of another codon encoding that same amino acid.

Representative codon-optimized coding regions encoding SEQ ID NO:4, optimized according to codon usage in humans designed by this method are presented herein as SEQ ID NO:23, SEQ ID NO:24, and SEQ ID NO:25.

In certain embodiments, the present invention provides an isolated polynucleotide comprising a nucleic acid fragment which encodes at least 10, at least 20, at least 30, at least 40, at least 50, at least 60, at least 70, at least 80, at least 90, at least 95, or at least 100 or more contiguous amino acids of SEQ ID NO:4, where the nucleic acid fragment is a fragment of a codon-optimized coding region encoding SEQ ID NO:4. The codon optimized coding region can be optimized according to codon usage in any species, for example any vertebrate species, for example any mammalian species, for example human.

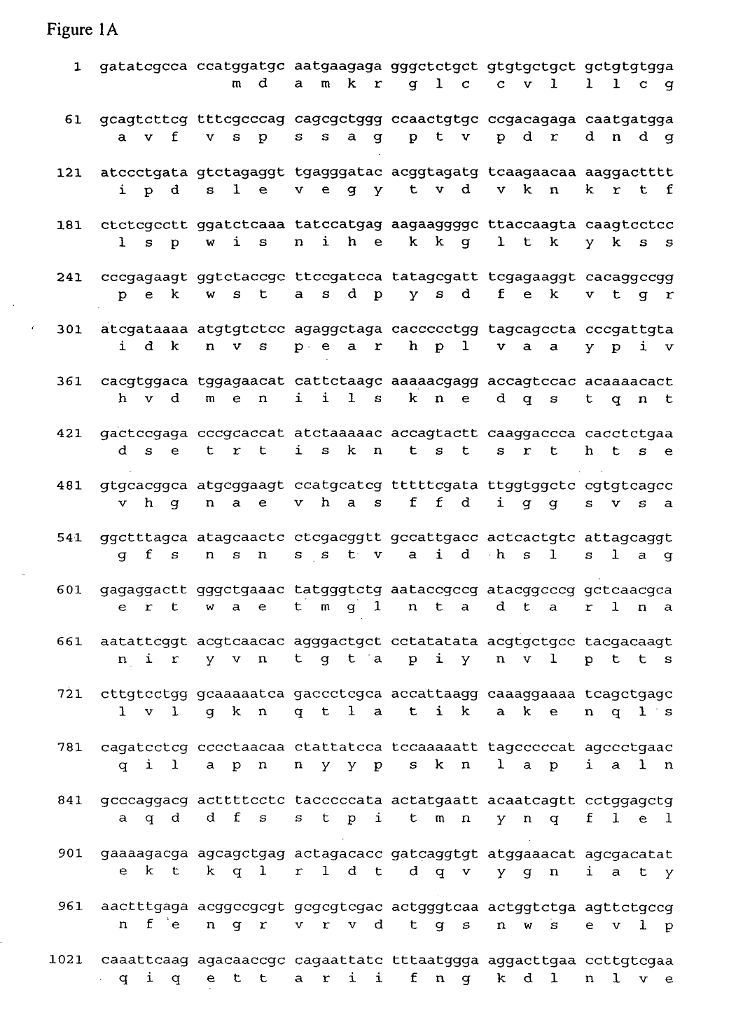

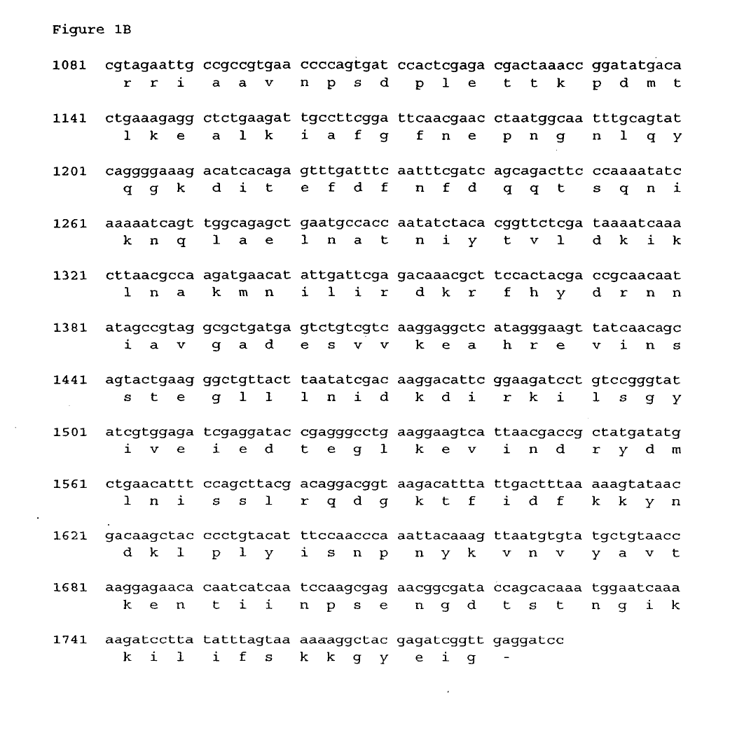

Further provided is an isolated polynucleotide comprising a nucleic acid fragment of a codon-optimized coding region encoding SEQ ID NO:4, where the nucleic acid fragment encodes amino acids 199 to 764 of SEQ ID NO:4. This polypeptide fragment is the 63-kD furin cleavage product (PA63) of the 82-kD protective antigen precursor polypeptide (PA83). The codon optimized coding region can be optimized according to codon usage in any species, for example any vertebrate species, for example any mammalian species, for example human. Included in this embodiment are nucleic acid fragments of a human codon-optimized coding region encoding SEQ ID NO:4, selected from: nucleotides 82 to 1779 of SEQ ID NO:1 (shown in FIG. 1), nucleotides 595 to 2292 of SEQ ID NO:23, nucleotides 595 to 2292 of SEQ ID NO:24, and nucleotides 595 to 2292 of SEQ ID NO:25.

Further provided is an isolated polynucleotide comprising a nucleic acid fragment of a codon-optimized coding region encoding SEQ ID NO:4, where the nucleic acid fragment encodes amino acids 30 to 764 of SEQ ID NO:4. This polypeptide fragment is the mature full-length PA, i.e., PA83. The codon optimized coding region can be optimized according to codon usage in any species, for example any vertebrate species, for example any mammalian species, for example human. Included in this embodiment are nucleic acid fragments of a human codon-optimized coding region encoding SEQ ID NO:4, selected from: nucleotides 88 to 2292 of SEQ ID NO:23, nucleotides 88 to 2292 of SEQ ID NO:24, and nucleotides 88 to 2292 of SEQ ID NO:25.

In certain embodiments, the present invention provides an isolated polynucleotide comprising a nucleic acid fragment which encodes a polypeptide at least 60%, 65%, 70%, 75%, 80%, 85%, 86%, 87%, 88%, 89%, 90%, 91%, 92%, 93%, 94%, 95%, 96%, 97%, 98%, or 99% identical to PA63, i.e., amino acids 199 to 764 of SEQ ID NO:4, and where the nucleic acid fragment is a variant of a codon-optimized coding region encoding SEQ ID NO:4. The codon optimized coding region can be optimized according to codon usage in any species, for example any vertebrate species, for example any mammalian species, for example human.

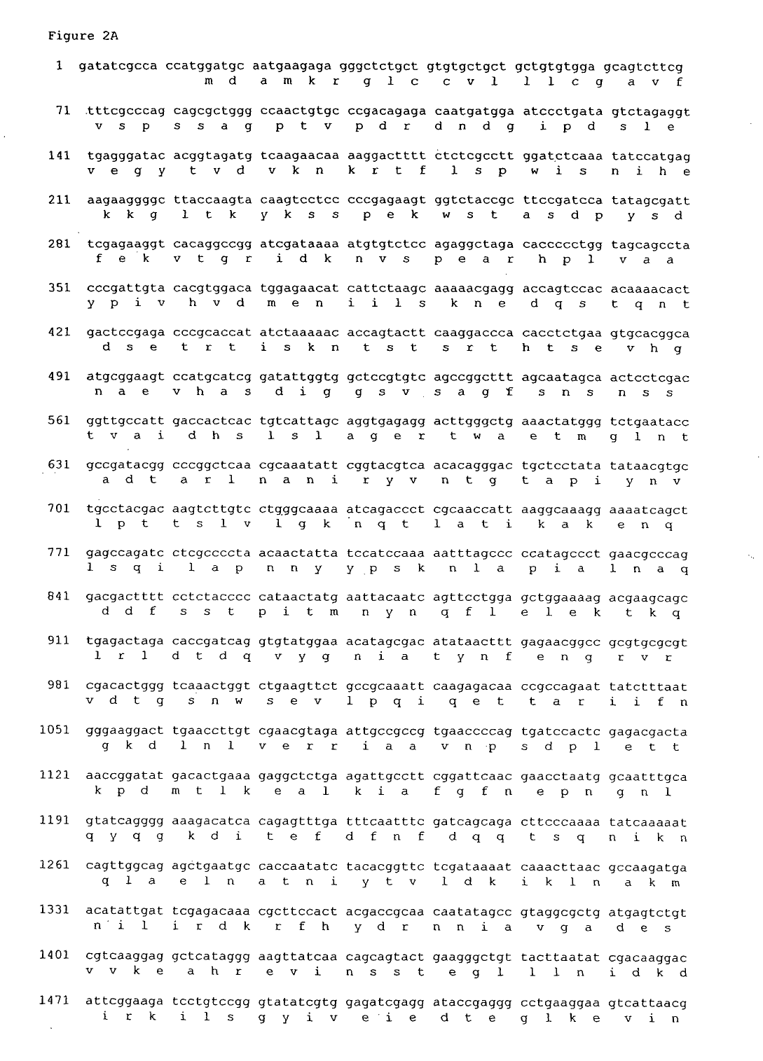

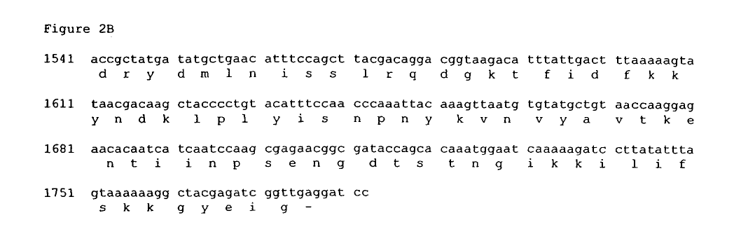

Further provided is an isolated polynucleotide comprising a nucleic acid fragment which encodes a polypeptide variant of PA63, i.e., amino acids 199 to 764 of SEQ ID NO:4, in which the amino acids corresponding to amino acids 342 and 343 of SEQ ID NO:4 have been deleted, and where the nucleic acid fragment is a variant of a codon-optimized coding region encoding SEQ ID NO:4. This variation in the amino acid sequence of PA63 eliminates two phenylalanine residues thought to be important in forming the pore in the B. anthracis lethal toxin. The codon optimized coding region can be optimized according to codon usage in any species, for example any vertebrate species, for example any mammalian species, for example human. Included in this embodiment is a nucleic acid fragment which is a variant of a human codon-optimized coding region encoding SEQ ID NO:4, where the nucleic acid fragment encodes amino acids 24 to 564 of SEQ ID NO:6 (shown in FIG. 2). Also included in this embodiment is a nucleic acid fragment comprising, or alternatively consisting of nucleotides 82 to 1773 of SEQ ID NO:5 (shown in FIG. 2).

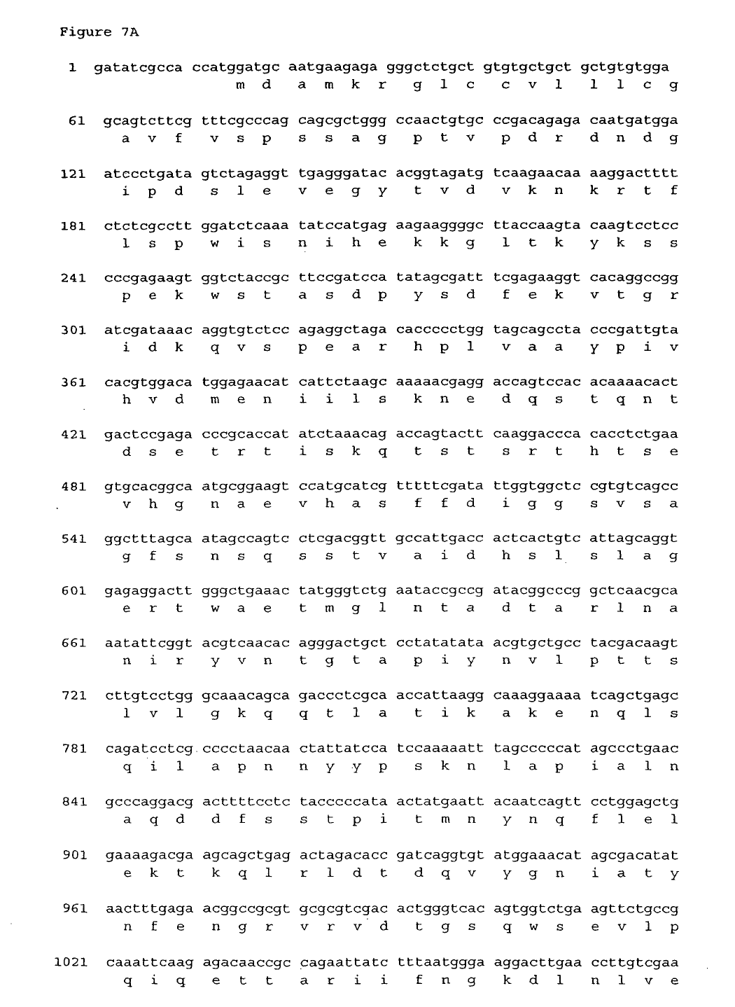

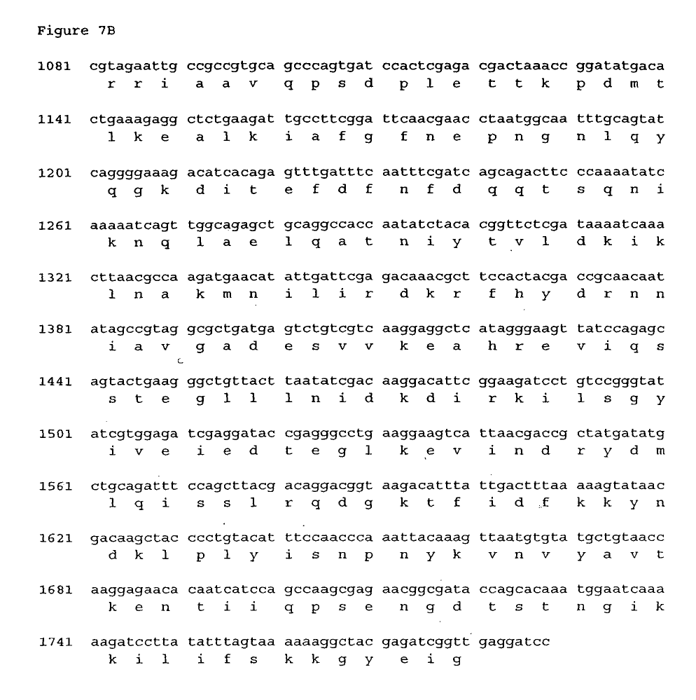

Further provided is an isolated polynucleotide comprising a nucleic acid fragment which encodes a polypeptide variant of PA63, i.e., amino acids 199 to 764 of SEQ ID NO:4, in which the asparagine residues at positions corresponding to amino acids 275, 321, 357, 417, 505, 538, 599, 650, 693, and 738 of SEQ ID NO:4 have been each replaced with an amino acids other than asparagine, and where the nucleic acid fragment is a variant of a codon-optimized coding region encoding SEQ ID NO:4. In certain embodiments, the asparagine residues at positions corresponding to amino acids 275, 321, 357, 417, 505, 538, 599, 650, 693, and 738 of SEQ ID NO:4 have been each replaced with glutamine residues, where the nucleic acid fragment is a variant of a codon-optimized coding region encoding SEQ ID NO:4. Either of these variations in the amino acid sequence of PA63 removes adventitous substrates for asparagine-linked glycosylation present in the amino acid sequence. The codon optimized coding region can be optimized according to codon usage in any species, for example any vertebrate species, for example any mammalian species, for example human. Included in this embodiment is a nucleic acid fragment which is a variant of a human codon-optimized coding region encoding SEQ ID NO:4, where the nucleic acid fragment encodes amino acids 24 to 566 of SEQ ID NO:18 (shown in FIG. 7). Also included in this embodiment is a nucleic acid fragment comprising, or alternatively consisting of nucleotides 82 to 1779 of SEQ ID NO:17 (shown in FIG. 7).

Further provided is an isolated polynucleotide comprising a nucleic acid fragment which encodes a polypeptide variant of PA63, i.e., amino acids 199 to 764 of SEQ ID NO:4, in which the amino acids corresponding to amino acids 342 and 343 of SEQ ID NO:4 have been deleted, where the asparagine residues at positions corresponding to amino acids 275, 321, 357, 417, 505, 538, 599, 650, 693, and 738 of SEQ ID NO:4 have been each replaced with an amino acids other than asparagine, for example, glutamine, and where the nucleic acid fragment is a variant of a codon-optimized coding region encoding SEQ ID NO:4. The codon optimized coding region can be optimized according to codon usage in any species, for example any vertebrate species, for example any mammalian species, for example human.

In certain embodiments, the present invention provides an isolated polynucleotide comprising a nucleic acid fragment which encodes a polypeptide at least 60%, 65%, 70%, 75%, 80%, 85%, 86%, 87%, 88%, 89%, 90%, 91%, 92%, 93%, 94%, 95%, 96%, 97%, 98%, or 99% identical to PA83, i.e., amino acids 30 to 764 of SEQ ID NO:4, and where the nucleic acid fragment is a variant of a codon-optimized coding region encoding SEQ ID NO:4. The codon optimized coding region can be optimized according to codon usage in any species, for example any vertebrate species, for example any mammalian species, for example human.

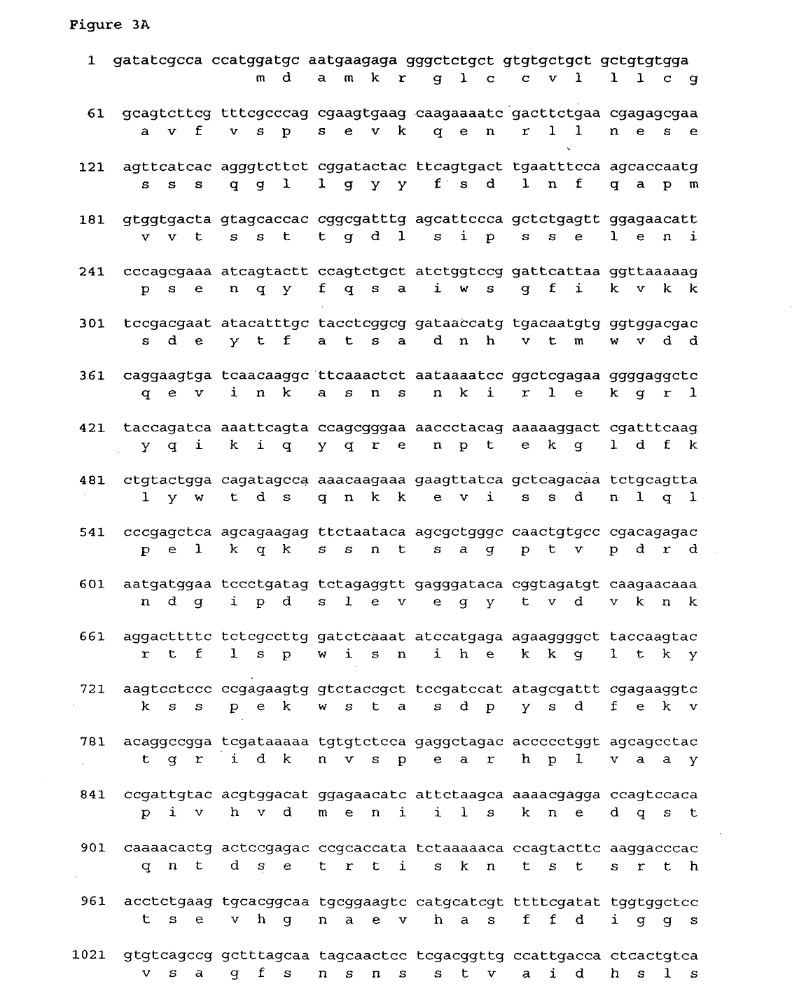

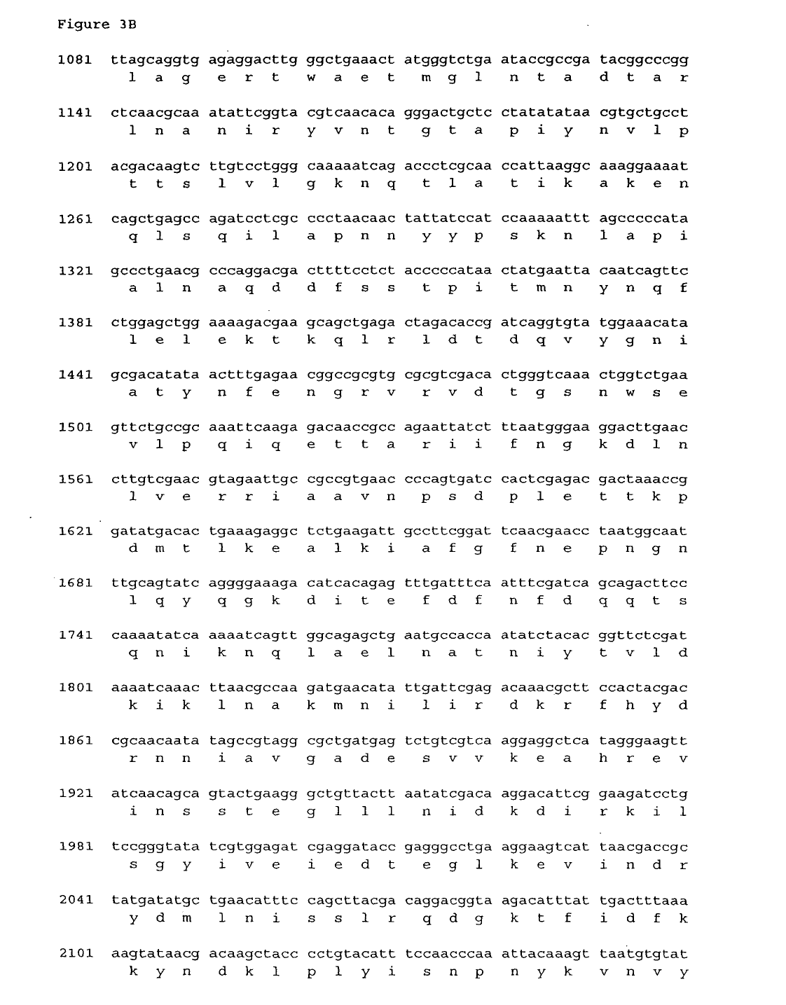

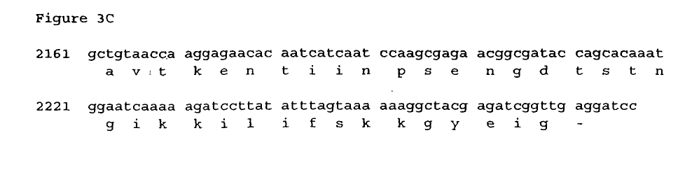

Further provided is an isolated polynucleotide comprising a nucleic acid fragment which encodes a polypeptide variant of PA83, i.e., amino acids 30 to 764 of SEQ ID NO:4, in which the amino acids corresponding to amino acids 192 to 197 of SEQ ID NO:4 have been deleted, and where the nucleic acid fragment is a variant of a codon-optimized coding region encoding SEQ ID NO:4. This variation in the amino acid sequence of PA83 eliminates the furin cleavage site in PA83, and thus the encoded polypeptide cannot be cleaved as a substrate for furin, and cannot form the pore of the lethal toxin of B. anthracis. The codon optimized coding region can be optimized according to codon usage in any species, for example any vertebrate species, for example any mammalian species, for example human. Included in this embodiment is a nucleic acid fragment which is a variant of a human codon-optimized coding region encoding SEQ ID NO:4, where the nucleic acid fragment encodes amino acids 24 to 752 of SEQ ID NO:8 (shown in FIG. 3). Also included in this embodiment is a nucleic acid fragment comprising, or alternatively consisting of nucleotides 82 to 2268 of SEQ ID NO:7 (shown in FIG. 3).

Further provided is an isolated polynucleotide comprising a nucleic acid fragment which encodes a polypeptide variant of PA83, i.e., amino acids 30 to 764 of SEQ ID NO:4, in which the asparagine residues at positions corresponding to amino acids 39, 153, 275, 321, 357, 417, 505, 538, 599, 650, 693, and 738 of SEQ ID NO:4 have been each replaced with an amino acids other than asparagine, and where the nucleic acid fragment is a variant of a codon-optimized coding region encoding SEQ ID NO:4. In certain embodiments, the asparagine residues at positions corresponding to amino acids 39, 153, 275, 321, 357, 417, 505, 538, 599, 650, 693, and 738 of SEQ ID NO:4 have been each replaced with glutamine residues, where the nucleic acid fragment is a variant of a codon-optimized coding region encoding SEQ ID NO:4. Either of these variations in the amino acid sequence of PA83 removes adventitous substrates for asparagine-linked glycosylation present in the amino acid sequence. The codon optimized coding region can be optimized according to codon usage in any species, for example any vertebrate species, for example any mammalian species, for example human.

Further provided is an isolated polynucleotide comprising a nucleic acid fragment which encodes a polypeptide variant of PA83, i.e., amino acids 30 to 764 of SEQ ID NO:4, in which the amino acids corresponding to amino acids 192 to 197 of SEQ ID NO:4 have been deleted, where the asparagine residues at positions corresponding to amino acids 39, 153, 275, 321, 357, 417, 505, 538, 599, 650, 693, and 738 of SEQ ID NO:4 have been each replaced with an amino acids other than asparagine, for example, glutamine, and where the nucleic acid fragment is a variant of a codon-optimized coding region encoding SEQ ID NO:4. The codon optimized coding region can be optimized according to codon usage in any species, for example any vertebrate species, for example any mammalian species, for example human.

The present invention provides isolated polynucleotides comprising codon-optimized coding regions of Bacillus anthracis LF, or fragments, variants, or derivatives thereof. In certain embodiments described herein, a codon-optimized coding region encoding SEQ ID NO:12 is optimized according to codon usage in humans (Homo sapiens). Alternatively, a codon-optimized coding region encoding SEQ ID NO:12 may be optimized according to codon usage in any plant, animal, or microbial species.

Codon-optimized coding regions encoding SEQ ID NO:12, optimized according to codon usage in humans are designed as follows. The amino acid composition of SEQ ID NO:12 is shown in Table 7.

TABLE 7

| | |
|---|---|
| Amino | Number in SEQ |
| Acid | ID NO: 12 |

| | | |
|---|---|---|
| A | Ala | 34 |
| R | Arg | 27 |
| C | Cys | 1 |
| G | Gly | 35 |
| H | His | 21 |
| I | Ile | 74 |
| L | Leu | 80 |
| K | Lys | 86 |
| M | Met | 10 |
| F | Phe | 29 |
| P | Pro | 21 |
| S | Ser | 54 |
| T | Thr | 28 |
| W | Trp | 5 |
| Y | Tyr | 35 |
| V | Val | 40 |
| N | Asn | 54 |
| D | Asp | 55 |
| Q | Gln | 41 |
| E | Glu | 79 |

Using the amino acid composition shown in Table 7, a human codon-optimized coding region which encodes SEQ ID NO:12 can be designed by any of the methods discussed herein. In the first approach, each amino acid is assigned the most frequent codon used in the human genome for that amino acid. According to this method, codons are assigned to the coding region encoding SEQ ID NO:4 as follows: the 29 phenylalanine codons are TTC, the 80 leucine codons are CTG, the 74 isoleucine codons are ATC, the 10 methionine codons are ATG, the 43 valine codons are GTG, the 54 serine codons are AGC, the 21 proline codons are CCC, the 28 threonine codons are ACC, the 34 alanine codons are GCC, the 35 tyrosine codons are TAC, the 21 histidine codons are CAC, the 41 glutamine codons are CAG, the 54 asparagine codons are AAC, the 86 lysine codons are AAG, the 55 aspartic acid codons are GAC, the 79 glutamic acid codons are GAG, the 5 tryptophan codons are TGG, the 27 arginine codons are CGG, AGA, or AGG (the frequencies of usage of these three codons in the human genome are not significantly different), and the 35 glycine codons are GGC. The codon-optimized LF coding region designed by this method is presented herein as SEQ ID NO:22.

Alternatively, a human codon-optimized coding region which encodes SEQ ID NO:12 can be designed by randomly assigning each of any given amino acid a codon based on the frequency that codon is used in the human genome. These frequencies are shown in Table 2 above. Using this latter method, codons are assigned to the coding region encoding SEQ ID NO:12 as follows: about 13 of the 29 phenylalanine codons are TTT and about 16 of the phenylalanine codons are TTC; about 6 of the 80 leucine codons are TTA, about 10 of the leucine codons are TTG, about 10 of the leucine codons are CTT, about 16 of the leucine codons are CTC, about 6 of the leucine codons are CTA, and about 32 of the leucine codons are CTG; about 26 of the 74 isoleucine codons are ATT, about 36 of the isoleucine codons are ATC, and about 12 of the isoleucine codons are ATA; the 10 methionine codons are ATG; about 7 of the 40 valine codons are GTT, about 9 of the valine codons are GTG, about 5 of the valine codons are GTA, and about 19 of the valine codons are GTG; about 10 of the 54 serine codons are TCT, about 12 of the serine codons are TCC, about 8 of the serine codons are TCA, about 3 of the serine codons are TCG, about 8 of the serine codons are AGT, and about 13 of the serine codons are AGC; about 6 of the 21 proline codons are CCT, about 7 of the proline codons are CCC, about 6 of the proline codons are CCA, and about 2 of the proline codons are CCG; about 7 of the 28 threonine codons are ACT, about 10 of the threonine codons are ACC, about 8 of the threonine codons are ACA, and about 3 of the threonine codons are ACG; about 9 of the 34 alanine codons are GGT, about 14 of the alanine codons are GCC, about 8 of the alanine codons are GCA, and about 3 of the alanine codons are GCG; about 15 of the 35 tyrosine codons are TAT and about 20 of the tyrosine codons are TAC; about 9 of the 21 histidine codons are CAT and about 12 of the histidine codons are CAC; about 10 of the 41 glutamine codons are CAA and about 31 of the glutamine codons are CAG; about 25 of the 54 asparagine codons are AAT and about 29 of the asparagine codons are AAC; about 36 of the 86 lysine codons are AAA and about 50 of the lysine codons are AAG; about 25 of the 55 aspartic acid codons are GAT and about 30 of the aspartic acid codons are GAC; about 33 of the 79 glutamic acid codons are GAA and about 46 of the glutamic acid codons are GAG; the single cysteine codon is either TGT or TGC; the 5 tryptophan codons are TGG; about 2 of the 27 arginine codons are CGT, about 5 of the arginine codons are CGC, about 3 of the arginine codons are CGA, about 6 of the arginine codons are CGG, about 6 of the arginine codons are AGA, and about 5 of the arginine codons are AGG; and about 6 of the 35 glycine codons are GGT, about 12 of the glycine codons are GGC, about 8 of the glycine codons are GGA, and about 9 of the glycine codons are GGG.

As described above, the term “about” means that the number of amino acids encoded by a certain codon may be one more or one less than the number given. It would be understood by those of ordinary skill in the art that the total number of any amino acid in the polypeptide sequence must remain constant, therefore, if there is one “more” of one codon encoding a give amino acid, there would have to be one “less” of another codon encoding that same amino acid.

Representative codon-optimized coding regions encoding SEQ ID NO:12, optimized according to codon usage in humans designed by this method are presented herein as SEQ ID NO:26, SEQ ID NO:27, and SEQ ID NO:28.

In certain embodiments, the present invention provides an isolated polynucleotide comprising a nucleic acid fragment which encodes at least 10, at least 20, at least 30, at least 40, at least 50, at least 60, at least 70, at least 80, at least 90, at least 95, or at least 100 or more contiguous amino acids of SEQ ID NO:12, where the nucleic acid fragment is a fragment of a codon-optimized coding region encoding SEQ ID NO:12. The codon optimized coding region can be optimized according to codon usage in any species, for example any vertebrate species, for example any mammalian species, for example human.

Further provided is an isolated polynucleotide comprising a nucleic acid fragment of a codon-optimized coding region encoding SEQ ID NO:12, where the nucleic acid fragment encodes amino acids 34 to 809 of SEQ ID NO:12. This polypeptide fragment is the mature form of B. anthracis LF. The codon optimized coding region can be optimized according to codon usage in any species, for example any vertebrate species, for example any mammalian species, for example human. Included in this embodiment are nucleic acid fragments of a human codon-optimized coding region encoding amino acids 34 to 809 of SEQ ID NO:12, selected from: nucleotides 100 to 2427 of SEQ ID NO:26, nucleotides 100 to 2427 of SEQ ID NO:27, and nucleotides 100 to 2427 of SEQ ID NO:28.

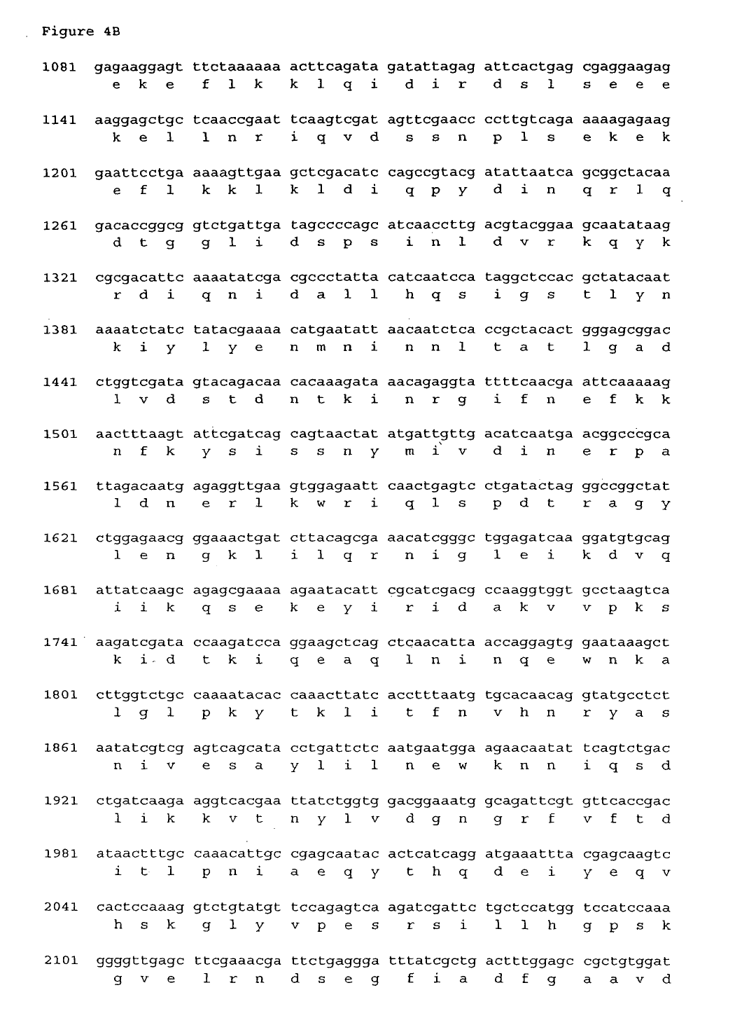

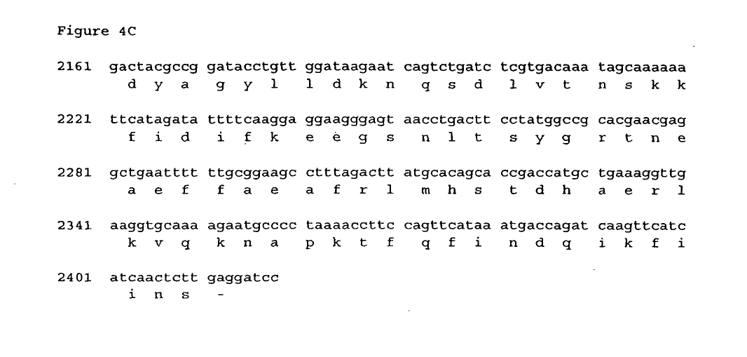

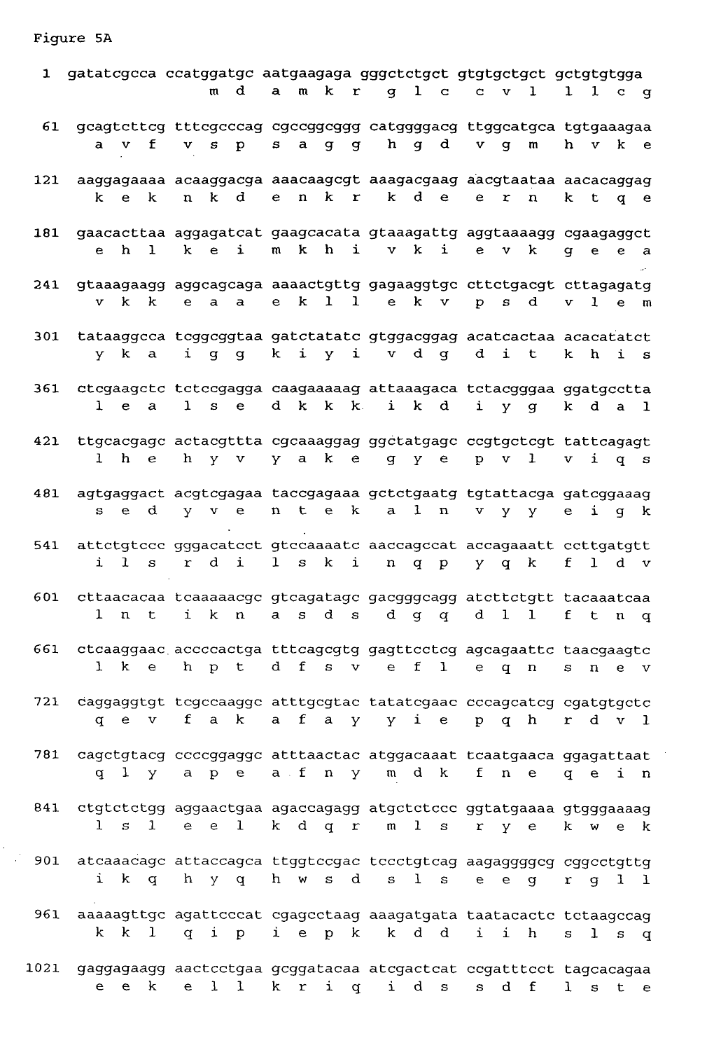

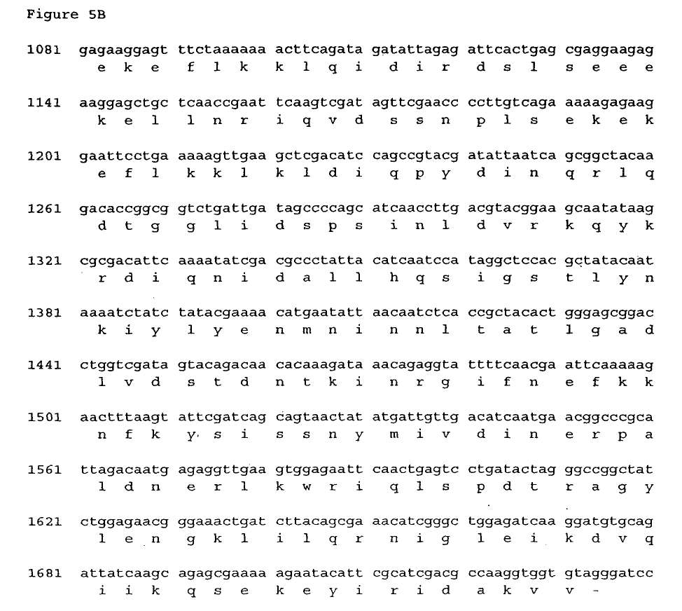

Further provided is an isolated polynucleotide comprising a nucleic acid fragment of a codon-optimized coding region encoding SEQ ID NO:12, where the nucleic acid fragment encodes amino acids 34 to 583 of SEQ ID NO:12. This polypeptide fragment encodes domains I-III of mature B. anthracis LF, but not domain IV, the protease domain. The codon optimized coding region can be optimized according to codon usage in any species, for example any vertebrate species, for example any mammalian species, for example human. Included in this embodiment are nucleic acid fragments of a human codon-optimized coding region encoding SEQ ID NO:12, selected from: nucleotides 82 to 1731 of SEQ ID NO:13 (shown in FIG. 5), nucleotides 100 to 1752 of SEQ ID NO:26, nucleotides 100 to 1752 of SEQ ID NO:27, and nucleotides 100 to 1752 of SEQ ID NO:28.

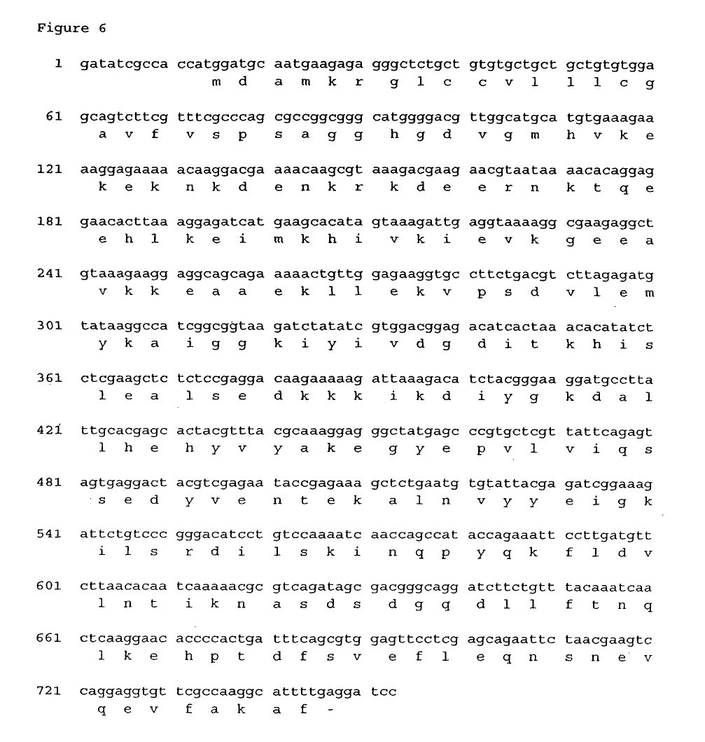

Further provided is an isolated polynucleotide comprising a nucleic acid fragment of a codon-optimized coding region encoding SEQ ID NO:12, where the nucleic acid fragment encodes amino acids 34 to 254 of SEQ ID NO:12. This polypeptide fragment encodes a portion of domain I of mature B. anthracis LF, that directly binds to PA63. The codon optimized coding region can be optimized according to codon usage in any species, for example any vertebrate species, for example any mammalian species, for example human. Included in this embodiment are nucleic acid fragments of a human codon-optimized coding region encoding SEQ ID NO:12, selected from: nucleotides 82 to 744 of SEQ ID NO:15 (shown in FIG. 6), nucleotides 100 to 762 of SEQ ID NO:26, nucleotides 100 to 762 of SEQ ID NO:27, and nucleotides 100 to 762 of SEQ ID NO:28.

In certain embodiments, the present invention provides an isolated polynucleotide comprising a nucleic acid fragment which encodes a polypeptide at least 60%, 65%, 70%, 75%, 80%, 85%, 86%, 87%, 88%, 89%, 90%, 91%, 92%, 93%, 94%, 95%, 96%, 97%, 98%, or 99% identical to LF, i.e., amino acids 34 to 809 of SEQ ID NO:12, and where the nucleic acid fragment is a variant of a codon-optimized coding region encoding SEQ ID NO:12. The codon optimized coding region can be optimized according to codon usage in any species, for example any vertebrate species, for example any mammalian species, for example human.

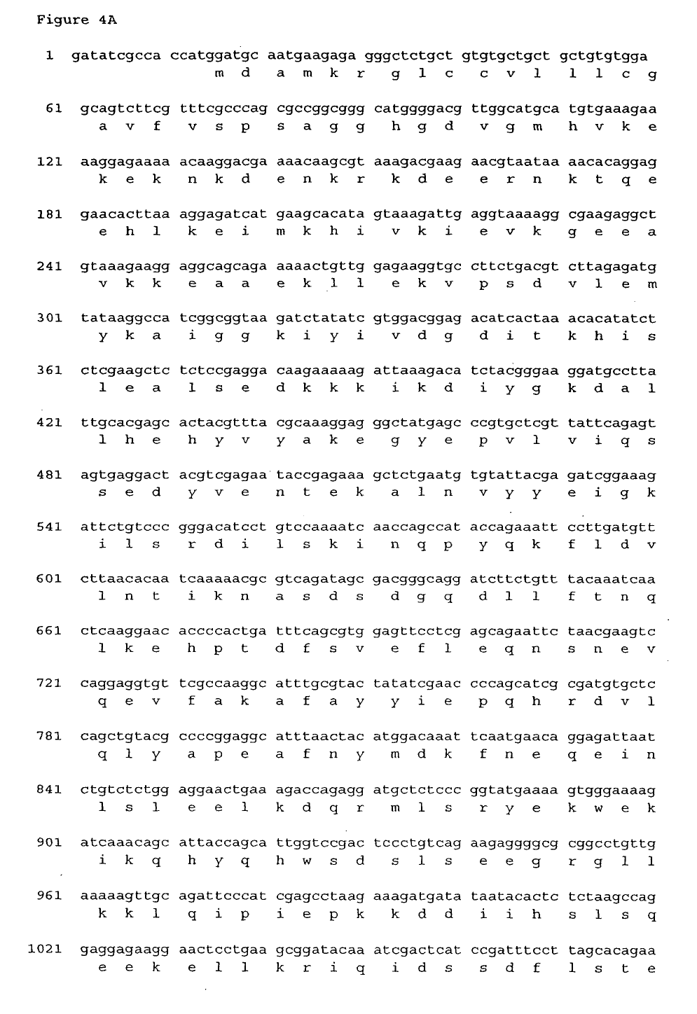

Further provided is an isolated polynucleotide comprising a nucleic acid fragment which encodes a polypeptide variant of LF, i.e., amino acids 34 to 809 of SEQ ID NO:12, in which the histidine residues corresponding to amino acids 719 and 723 of SEQ ID NO:12 have been deleted, and replaced with an amino acid other than histidine, and/or the glutamic acid residue corresponding to amino acid 720 of SEQ ID NO:12 has been deleted and replaced with an amino acid other than glutamic acid, where the nucleic acid fragment is a variant of a codon-optimized coding region encoding SEQ ID NO:12. In certain embodiments, the histidine residues corresponding to amino acids 719 and 723 of SEQ ID NO:12 have been deleted, and replaced with alanine residues, and/or the glutamic acid residue corresponding to amino acid 720 of SEQ ID NO:12 has been deleted and replaced with an aspartic acid residue, where the nucleic acid fragment is a variant of a codon-optimized coding region encoding SEQ ID NO:12. Any of these variations in the amino acid sequence of LF, either alone or in combination, eliminate the protease activity of LF. The codon optimized coding region can be optimized according to codon usage in any species, for example any vertebrate species, for example any mammalian species, for example human. Included in this embodiment is a nucleic acid fragment which is a variant of a human codon-optimized coding region encoding SEQ ID NO:12, where the nucleic acid fragment encodes amino acids 24 to 799 of SEQ ID NO:10 (shown in FIG. 4). Also included in this embodiment is a nucleic acid fragment comprising, or alternatively consisting of nucleotides 82 to 2409 of SEQ ID NO:9 (shown in FIG. 4).

Further provided is an isolated polynucleotide comprising a nucleic acid fragment which encodes a polypeptide variant of LF, i.e., amino acids 34 to 809 of SEQ ID NO:12, in which the asparagine residues at positions corresponding to amino acids 62, 212, 286, 478, 712, 736, and 757 of SEQ ID NO:12 have been each replaced with an amino acids other than asparagine, and where the nucleic acid fragment is a variant of a codon-optimized coding region encoding SEQ ID NO:12. In certain embodiments, the asparagine residues at positions corresponding to amino acids 62, 212, 286, 478, 712, 736, and 757 of SEQ ID NO:12 have been each replaced with glutamine residues, where the nucleic acid fragment is a variant of a codon-optimized coding region encoding SEQ ID NO:12. Either of these variations in the amino acid sequence of LF remove adventitous substrates for asparagine-linked glycosylation present in the amino acid sequence. The codon optimized coding region can be optimized according to codon usage in any species, for example any vertebrate species, for example any mammalian species, for example human.

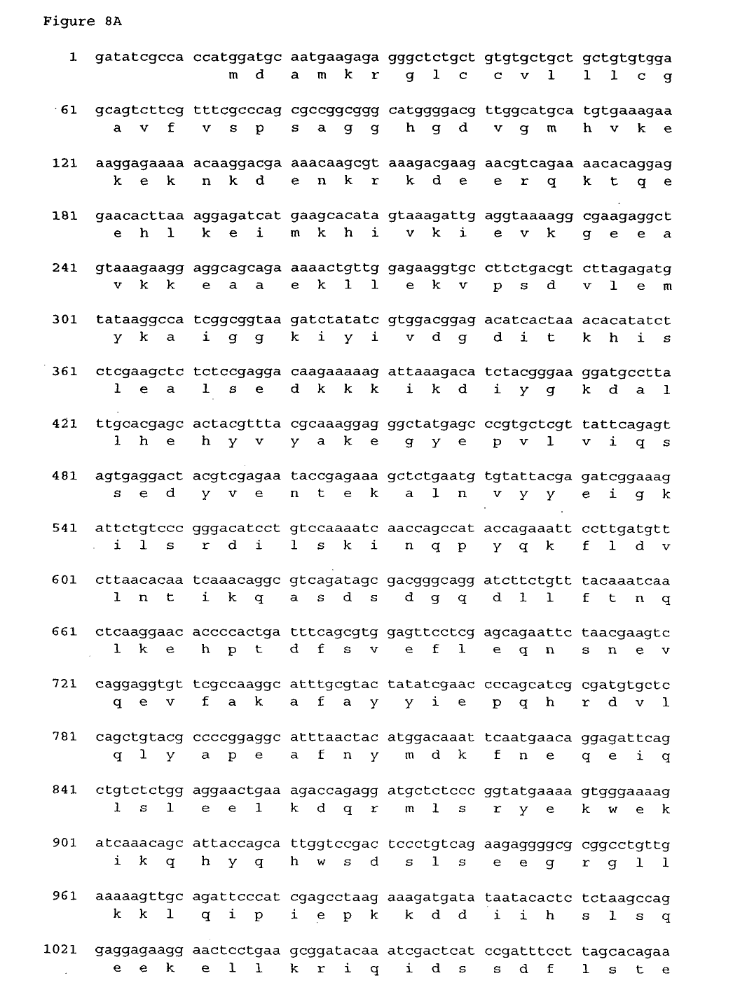

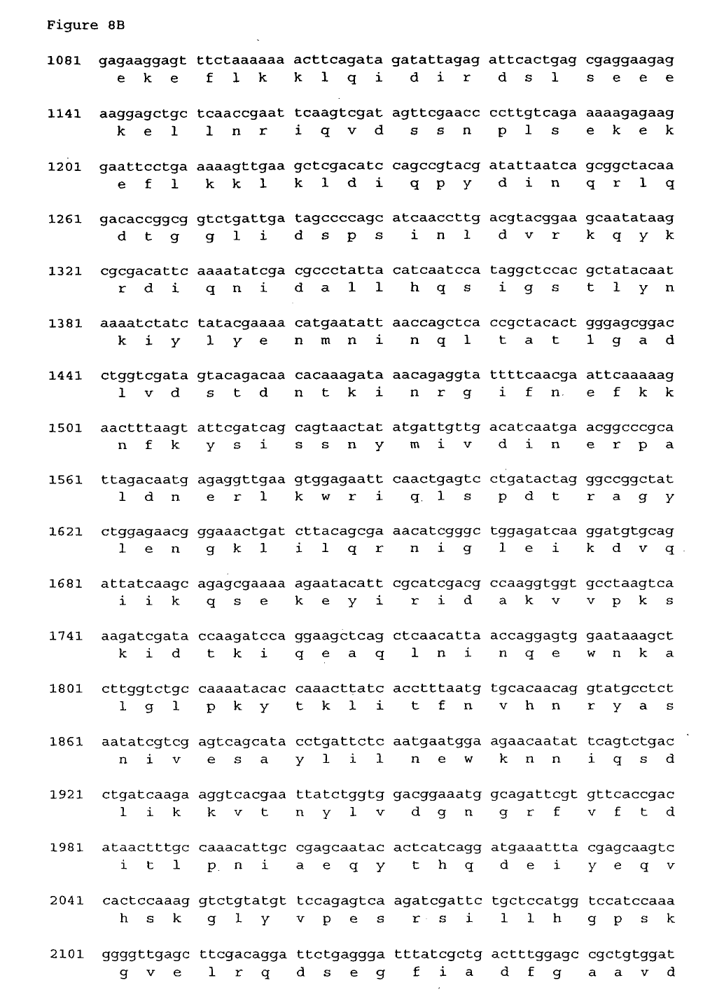

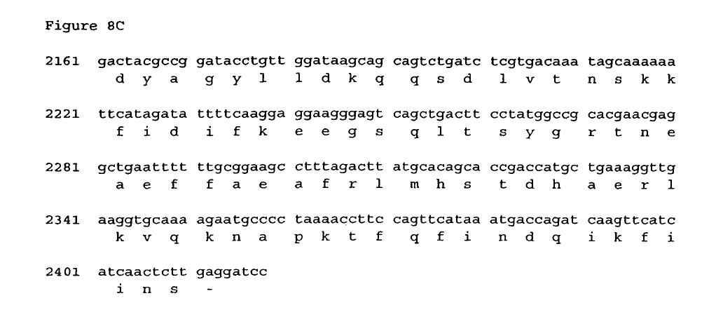

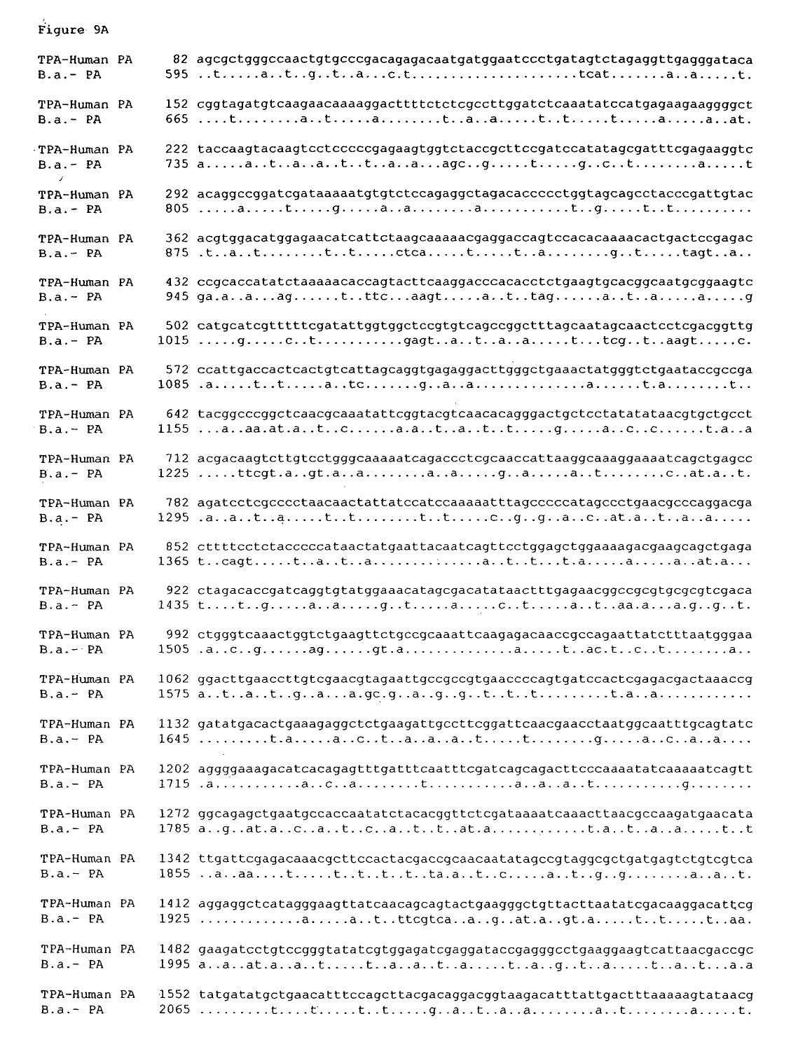

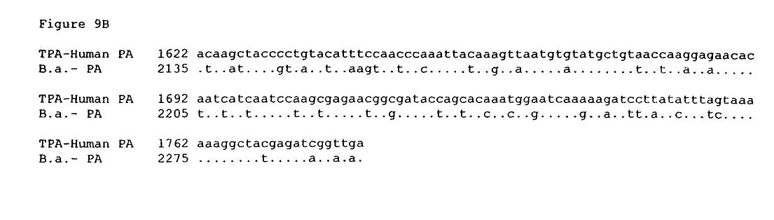

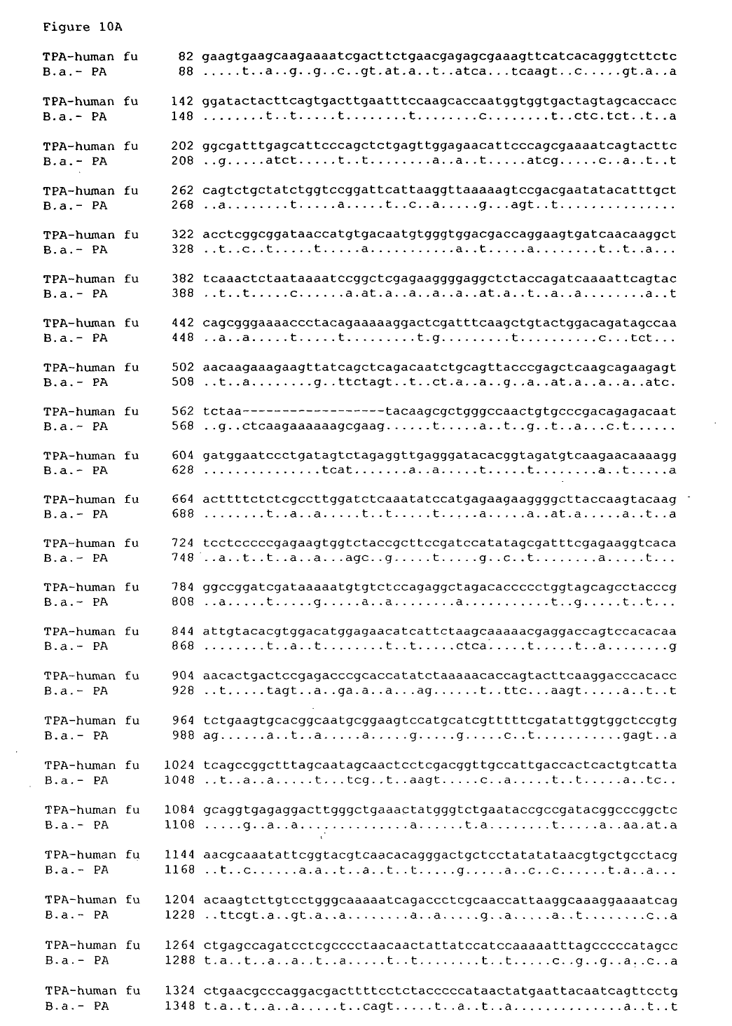

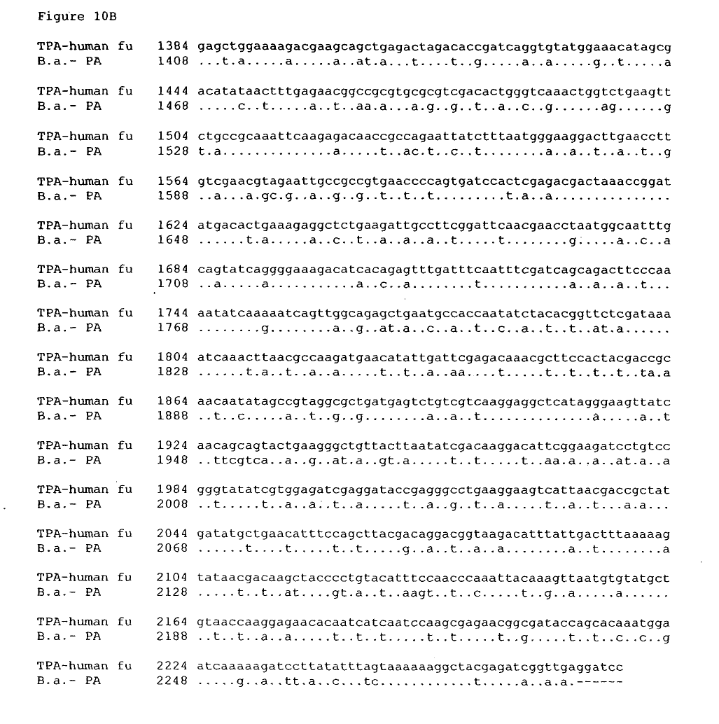

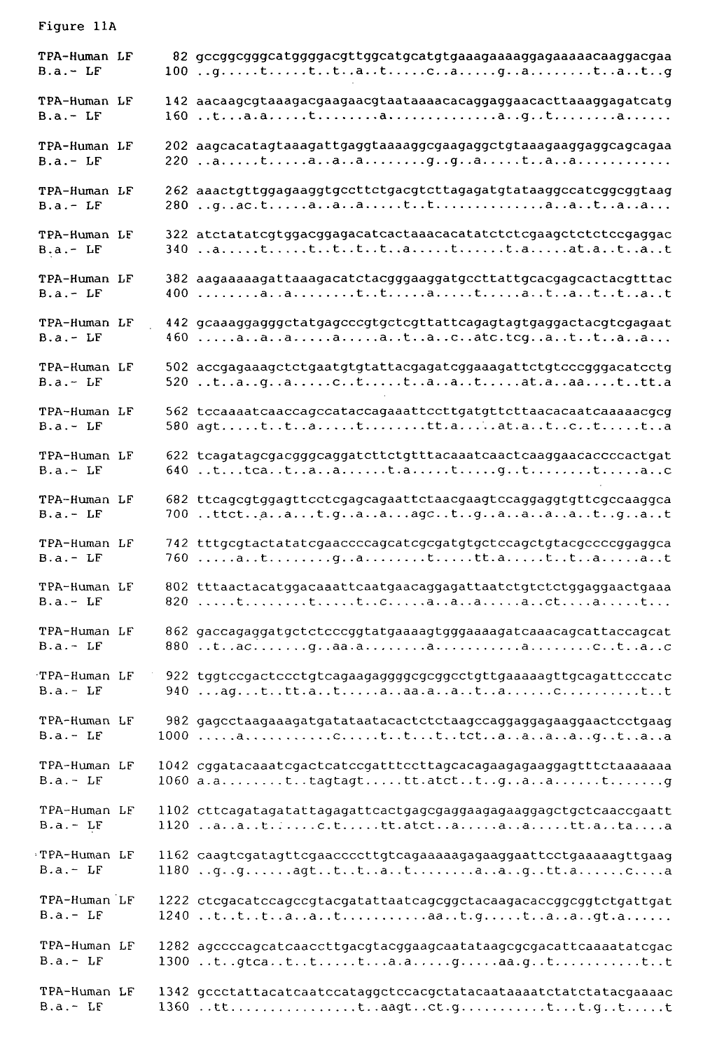

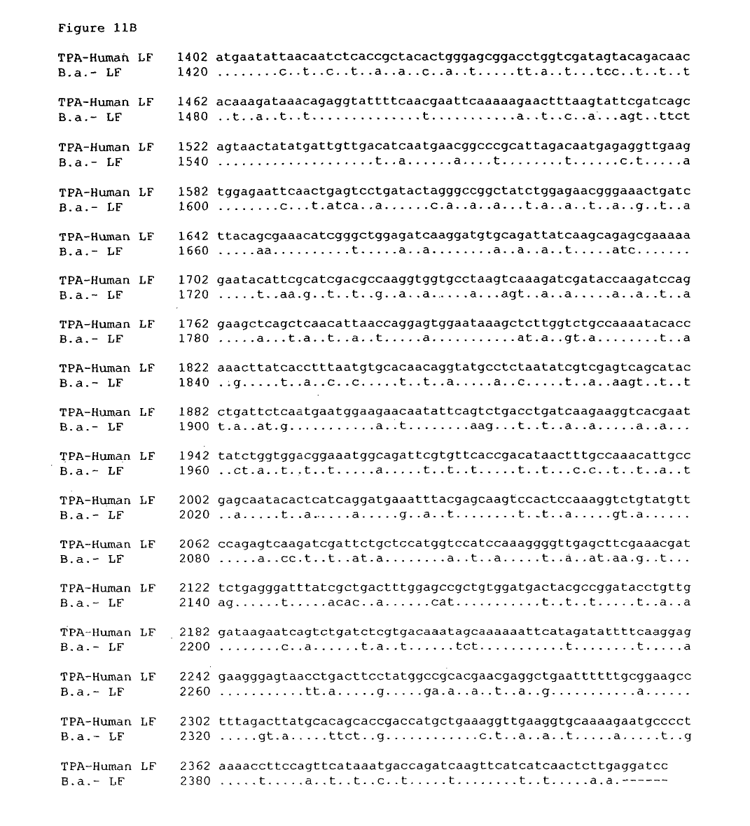

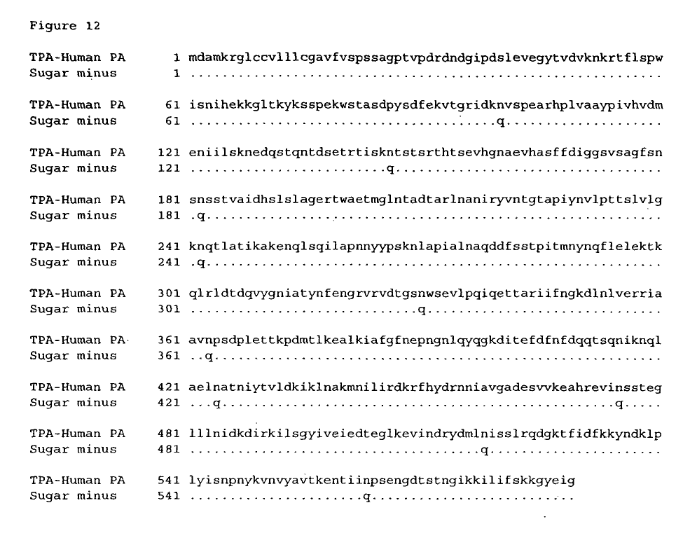

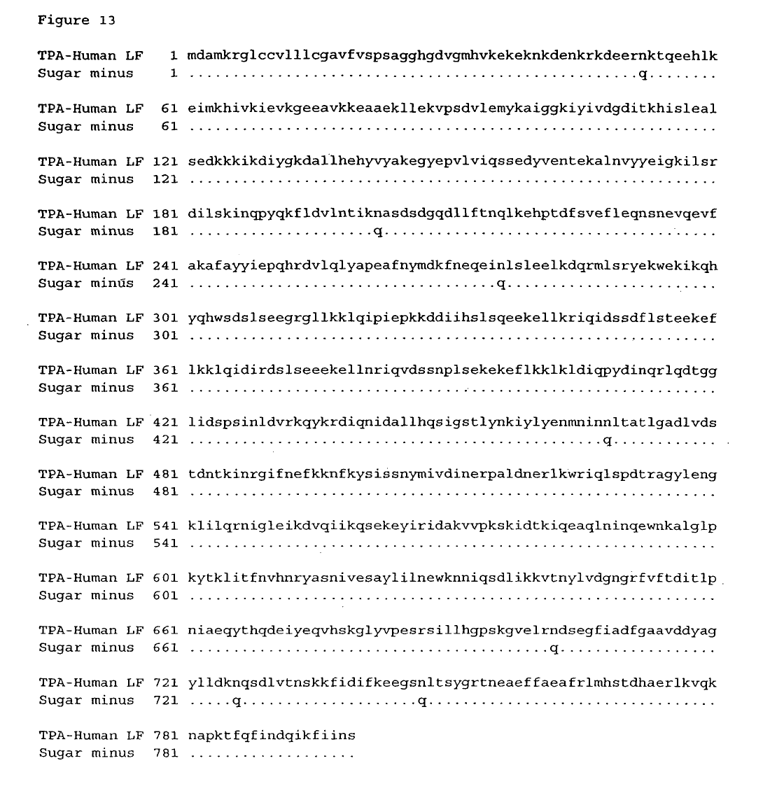

Further provided is an isolated polynucleotide comprising a nucleic acid fragment which encodes a polypeptide variant of LF, i.e., amino acids 34 to 809 of, SEQ ID NO:12, in which the histidine residues corresponding to amino acids 719 and 723 of SEQ ID NO:12 have been deleted, and replaced with an amino acid other than histidine, and/or the glutamic acid residue corresponding to amino acid 720 of SEQ ID NO:12 has been deleted and replaced with an amino acid other than glutamic acid, and the asparagine residues at positions corresponding to amino acids 62, 212, 286, 478, 712, 736, and 757 of SEQ ID NO:12 have been each replaced with an amino acids other than asparagine, and where the nucleic acid fragment is a variant of a codon-optimized coding region encoding SEQ ID NO:12. In certain embodiments, the histidine residues corresponding to amino acids 719 and 723 of SEQ ID NO:12 have been deleted, and replaced with alanine residues, and/or the glutamic acid residue corresponding to amino acid 720 of SEQ ID NO:12 has been deleted and replaced with an aspartic acid residue, and the asparagine residues at positions corresponding to amino acids 62, 212, 286, 478, 712, 736, and 757 of SEQ ID NO:12 have been each replaced with glutamine residues, where the nucleic acid fragment is a variant of a codon-optimized coding region encoding SEQ ID NO:12. Any of these variations in the amino acid sequence of LF, either alone or in combination, eliminate the protease activity of LF, and also, adventitous substrates for asparagine-linked glycosylation present in the amino acid sequence have been removed. The codon optimized coding region can be optimized according to codon usage in any species, for example any vertebrate species, for example any mammalian species, for example human. Included in this embodiment is a nucleic acid fragment which is a variant of a human codon-optimized coding region encoding SEQ ID NO:12, where the nucleic acid fragment encodes amino acids 24 to 799 of SEQ ID NO:20 (shown in FIG. 8). Also included in this embodiment is a nucleic acid fragment comprising, or alternatively consisting of nucleotides 82 to 2409 of SEQ ID NO:19 (shown in FIG. 8).

In this manner, the present invention provides a method of enhancing the level of polypeptide expression from delivered polynucleotides in vivo and/or facilitating uptake of the polynucleotides by the cells of a desired species, for example a vertebrate species, for example a mammalian species, for example humans. Accordingly, the present invention provides a method of treatment and prevention against Bacillus anthracis infection.

Methods and Administration

The present invention further provides methods for delivering a polypeptide into a vertebrate, which comprise administering to a vertebrate one or more of the compositions described herein; such that upon administration of compositions such as those described herein, a B. anthracis polypeptide is expressed in the vertebrate, in an amount sufficient generate an immune response to B. anthracis.

The term “vertebrate” is intended to encompass a singular “vertebrate” as well as plural “vertebrates,” and comprises mammals and birds, as well as fish, reptiles, and amphibians.

The term “mammal” is intended to encompass a singular “mammal” and plural “mammals,” and includes, but is not limited to humans; primates such as apes, monkeys, orangutans, and chimpanzees; canids such as dogs and wolves; felids such as cats, lions, and tigers; equids such as horses, donkeys, and zebras, food animals such as cows, pigs, and sheep; ungalates such as deer and giraffes; and ursids such as bears. In particular, the mammal can be a human subject, a food animal or a companion animal.

The present invention further provides a method for generating, enhancing or modulating an immune response to B. anthracis comprising administering to a vertebrate one or more of the compositions described herein. In this method, the composition includes an isolated polynucleotide comprising a human codon-optimized coding region encoding a polypeptide of Bacillus anthracis, or a nucleic acid fragment of such a coding region encoding a fragment, variant, or derivative thereof. The polynucleotides are incorporated into the cells of the vertebrate in vivo, and an antigenic amount of the Bacillus anthracis polypeptide, or fragment, variant, or derivative thereof, is produced in vivo. Upon administration of the composition according to this method, the Bacillus anthracis polypeptide is expressed in the vertebrate in an amount sufficient to elicit an immune response. Such an immune response might be used, for example, to generate antibodies to B. anthracis for use in diagnostic assays or as laboratory reagents.

The present invention further provides a method for generating, enhancing, or modulating a protective and/or therapeutic immune response to B. anthracis in a vertebrate, comprising administering to a vertebrate in need of therapeutic and/or preventative immunity one or more of the compositions described herein. In this method, the composition includes an isolated polynucleotide comprising a human codon-optimized coding region encoding a polypeptide of Bacillus anthracis, or a nucleic acid fragment of such a coding region encoding a fragment, variant, or derivative thereof. The polynucleotides are incorporated into the cells of the vertebrate in vivo, and an immunologically effective amount of the Bacillus anthracis polypeptide, or fragment or variant is produced in vivo. Upon administration of the composition according to this method, the Bacillus anthracis polypeptide is expressed in the vertebrate in a therapeutically or prophylactically effective amount.

As used herein, an “immune response” refers to the ability of a vertebrate to elicit an immune reaction to a composition delivered to that vertebrate. Examples of immune responses include an antibody response or a cellular, e.g., T-cell, response. One or more compositions of the present invention may be used to treat a vertebrate prophylactically, e.g. as a prophylactic vaccine, to establish or enhance immunity to B. anthracis in a healthy vertebrate prior to exposure to B. anthracis or contraction of anthrax disease, thus preventing the disease or reducing the severity of disease symptoms. One or more compositions of the present invention may also be used to treat a vertebrate already exposed to B. anthracis, or already suffering from anthrax disease to further stimulate the immune system of the vertebrate, thus reducing or eliminating the symptoms associated with that disease or disorder. As defined herein, “treatment of a vertebrate” refers to the use of One or more compositions of the present invention to prevent, cure, retard, or reduce the severity of anthrax disease symptoms in a vertebrate, and/or result in no worsening of anthrax disease over a specified period of time. It is not required that any composition of the present invention provide total immunity to B. anthracis or totally cure or eliminate all anthrax disease symptoms. As used herein, a “a vertebrate in need of therapeutic and/or preventative immunity” refers to a vertebrate which it is desirable to treat, i.e., to prevent, cure, retard, or reduce the severity of anthrax disease symptoms, and/or result in no worsening of anthrax disease over a specified period of time.

In other embodiments, one or more compositions of the present invention are utilized in a “prime boost” regimen. In these embodiments, one or more polynucleotide vaccine compositions of the present invention are delivered to a vertebrate, thereby priming the immune response of the vertebrate to B. anthracis, and then a second immunogenic composition is utilized as a boost vaccination. One or more polynucleotide vaccine compositions of the present invention are used to prime immunity, and then a second immunogenic composition, e.g., a recombinant viral vaccine or vaccines, a different polynucleotide vaccine, one or more purified subunit Bacillus anthracis proteins, e.g., PA or LF or a variant, fragment, or derivative thereof, or the existing AVA anthrax vaccine, is used to boost the anti-Bacillus anthracis immune response. The polynucleotide vaccine compositions may comprise one or more vectors for expression of one or more Bacillus anthracis lethal toxin genes as described herein. In addition, polynucleotide prime vaccine and the later boost vaccine elicit an immune response to the same or similar antigens, or they may be to different antigens.

In another embodiment, vectors are prepared for expression in the recombinant virus vaccine and in transfected mammalian cells as part of a polynucleotide vaccine.

The terms “priming” or “primary” and “boost” or “boosting” are used herein to refer to the initial and subsequent immunizations, respectively, i.e., in accordance with the definitions these terms normally have in immunology.

Sterile immunity is defined herein as the ability to completely inhibit the germination of anthrax spores into bacteria. If germination occurs, the bacteria produce Letx and surviving rabbits immunized against the PA antigen would be expected to generate a response to LF. Likewise, rabbits immunized with LF should have a measurable response to PA.

Antibodies induced by recombinant PA or by the commercial anthrax vaccine, AVA, have been shown to have potential activities other than neutralization, that may affect the outcome of an infection by anthrax. Among these potential activities is the effect of preventing germination of bacteria from the spores. (Welkos, S. et al. Microbiology. 147: 1677-85 (2001)). DNA vaccination may induce levels of antibody consistent with those that prevent germination. The absence of an increase in LF, PA, or neutralization titers, following infection, has been observed in animals vaccinated with DNA vaccines. This is in contrast to animals vaccinated twice with a commercial anthrax vaccine, AVA. While not being bound by theory, the DNA vaccine may induce antibodies that possess novel protective activities independent of lethal toxin neutralization.

In certain embodiments, one or more compositions of the present invention are delivered to a vertebrate by methods described herein, thereby achieving an effective immune response, and or an effective therapeutic or preventative immune response.

More specifically, the compositions of the present invention may be administered to any tissue of a vertebrate, including, but not limited to, muscle, skin, brain tissue, lung tissue, liver tissue, spleen tissue, bone marrow tissue, thymus tissue, heart tissue, e.g., myocardium, endocardium, and pericardium, lymph tissue, blood tissue, bone tissue, pancreas tissue, kidney tissue, gall bladder tissue, stomach tissue, intestinal tissue, testicular tissue, ovarian tissue, uterine tissue, vaginal tissue, rectal tissue, nervous system tissue, eye tissue, glandular tissue, tongue tissue, and connective tissue, e.g., cartilage.

Furthermore, the compositions of the present invention may be administered to any internal cavity of a vertebrate, including, but not limited to, the lungs, the mouth, the nasal cavity, the stomach, the peritoneal cavity, the intestine, any heart chamber, veins, arteries, capillaries, lymphatic cavities, the uterine cavity, the vaginal cavity, the rectal cavity, joint cavities, ventricles in brain, spinal canal in spinal cord, the ocular cavities, the lumen of a duct of a salivary gland or a liver. When the compositions of the present invention is administered to the lumen of a duct of a salivary gland or a liver, the desired polypeptide is encoded in each of the salivary gland and the liver such that the polypeptide is delivered into the blood stream of the vertebrate from each of the salivary gland and the liver. Certain modes for administration to secretory organs of a gastrointestinal system using the salivary gland, liver and pancreas to release a desired polypeptide into the bloodstream is disclosed in U.S. Pat. Nos. 5,837,693 and 6,004,944, both of which are incorporated herein by reference in their entireties.

In one embodiment, the compositions are administered to muscle, either skeletal muscle or cardiac muscle, or lung tissue. Specific, but non-limiting modes for administration to lung tissue are disclosed in Wheeler, C. J., et al., Proc. Natl. Acad. Sci. USA 93:11454-11459 (1996), which is incorporated herein by reference in its entirety.

According to the disclosed methods, compositions of the present invention can be administered by intramuscular (i.m.), subcutaneous (s.c.), or intrapulmonary routes. Other suitable routes of administration include, but not limited to intratracheal, transdermal, intraocular, intranasal, inhalation, intracavity, intravenous (i.v.), intraductal (e.g., into the pancreas) and intraparenchymal (i.e., into any tissue) administration. Transdermal delivery includes, but not limited to intradermal (e.g., into the dermis or epidermis), transdermal (e.g., percutaneous) and transmucosal administration (i.e., into or through skin or mucosal tissue). Intracavity administration includes, but not limited to administration into oral, vaginal, rectal, nasal, peritoneal, or intestinal cavities as well as, intrathecal (i.e., into spinal canal), intraventricular (i.e., into the brain ventricles or the heart ventricles), intraatrial (i.e., into the heart atrium) and sub arachnoid (i.e., into the sub arachnoid spaces of the brain) administration.

Any mode of administration can be used so long as the mode results in the expression of the desired peptide or protein, in the desired tissue, in an amount sufficient to generate an immune response to B. anthracis and/or to generate a prophylactically or therapeutically effective immune response to B. anthracis in a vertebrate in need of such response. Administration means of the present invention include needle injection, catheter infusion, biolistic injectors, particle accelerators (e.g., “gene guns” or pneumatic “needleless” injectors) Med-E-Jet (Vahlsing, H., et al., J. Immunol. Methods 171, 11-22 (1994)), Pigjet (Schrijver, R., et al., Vaccine 15, 1908-1916 (1997)), Biojector (Davis, H., et al., Vaccine 12, 1503-1509 (1994); Gramzinski, R., et al., Mol. Med. 4, 109-118 (1998)), AdvantaJet (Linmayer, I., et al., Diabetes Care 9:294-297 (1986)), Medi-jector (Martins, J., and Roedl, E. J. Occup. Med. 21:821-824 (1979)), gelfoam sponge depots, other commercially available depot materials (e.g., hydrogels), osmotic pumps (e.g., Alza minipumps), oral or suppositorial solid (tablet or pill) pharmaceutical formulations, topical skin creams, and decanting, use of polynucleotide coated suture (Qin, Y., et al., Life Sciences 65, 2193-2203 (1999)) or topical applications during surgery. Certain modes of administration are intramuscular needle-based injection and pulmonary application via catheter infusion. Each of the references cited in this paragraph is incorporated herein by reference in its entirety.

Determining an effective amount of one or more compositions of the present invention depends upon a number of factors including, for example, the antigen being expressed, e.g., PA or LF or fragments, variants, or derivatives thereof, the age and weight of the subject, the precise condition requiring treatment and its severity, and the route of administration. Based on the above factors, determining the precise amount, number of doses, and timing of doses are within the ordinary skill in the art and will be readily determined by the attending physician or veterinarian.

Compositions of the present invention may include various salts, excipients, delivery vehicles and/or auxilliary agents as are disclosed, e.g., in U.S. patent application Publication 2002/0019358, published Feb. 14, 2002, which is incorporated herein by reference in its entirety.

Furthermore, compositions of the present invention may include one or more transfection facilitating compounds that facilitate delivery of polynucleotides to the interior of a cell, and/or to a desired location within a cell. As used herein, the terms “transfection faciliating compound,” “transfection facilitating agent,” and “transfection faciliating material” are synonymous, and may be used interchangeably. It should be noted that certain transfection facilitating compounds may also be “adjuvants” as described infra, i.e., in addition to facilitating delivery of polynucleotides to the interior of a cell, the compound acts to alter or increase the immune response to the antigen encoded by that polynucleotide. Examples of the transfection facilitating compounds include, but are not limited to inorganic materials such as calcium phosphate, alum (aluminum sulfate), and gold particles (e.g., “powder” type delivery vehicles); peptides that are, for example, cationic, intercell targeting (for selective delivery to certain cell types), intracell targeting (for nucleor localization or endosomal escape), and ampipathic (helix forming or pore forming); proteins that are, for example, basic (e.g., positively charged) such as histones, targeting (e.g., asialoprotein), viral (e.g., Sendai virus coat protein), and pore-forming; lipids that are, for example, cationic (e.g., DMRIE, DOSPA, DC-Chol), basic (e.g., steryl amine), neutral (e.g., cholesterol), anionic (e.g., phosphatidyl serine), and zwitterionic (e.g., DOPE, DOPC); and polymers such as dendrimers, star-polymers, “homogenous” poly-amino acids (e.g., poly-lysine, poly-arginine), “heterogenous” poly-amino acids (e.g., mixtures of lysine & glycine), co-polymers, polyvinylpyrrolidinone (PVP), and polyethylene glycol (PEG). A transfection facilitating material can be used alone or in combination with one or more other transfection facilitating materials. Two or more transfection facilitating materials can be combined by chemical bonding (e.g., covalent and ionic such as in lipidated polylysine, PEGylated polylysine) (Toncheva, et al., Biochim. Biophys. Acta 1380(3):354-368 (1988)), mechical mixing (e.g., free moving materials in liquid or solid phase such as “polylysine+cationic lipids”) (Gao and Huang, Biochemistry 35:1027-1036 (1996); Trubetskoy, et al., Biochem. Biophys. Acta 1131:311-313 (1992)), and aggregation (e.g., co-precipitation, gel forming such as in cationic lipids+poly-lactide co-galactide, and polylysine+gelatin).

One category of transfection facilitating materials is cationic lipids. Examples of cationic lipids are 5-carboxyspermylglycine dioctadecylamide (DOGS) and dipalmitoyl-phophatidylethanolamine-5carboxyspermylamide (DPPES). Cationic cholesterol derivatives are also useful, including {3β-[N—N′,N′-dimethylamino)ethane]-carbomoyl}-cholesterol (DC-Chol). Dimethyldioctdecyl-ammonium bromide (DDAB), N-(3-aminopropyl)-N,N-(bis-(2-tetradecyloxyethyl))-N-methyl-ammonium bromide (PA-DEMO), N-(3-aminopropyl)-N,N-(bis-(2-dodecyloxyethyl))-N-methyl-ammonium bromide (PA-DELO), N,N,N-tris-(2-dodecyloxy)ethyl-N-(3-amino)propyl-ammonium bromide (PA-TELO), and N¹-(3-aminopropyl)((2-dodecyloxy)ethyl)-N²-(2-dodecyloxy)ethyl-1-piperazinaminium bromide (GA-LOE-BP) can also be employed in the present invention.

Non-diether cationic lipids, such as DL-1,2-dioleoyl-3-dimethylaminopropyl-β-hydroxyethylammonium (DORI diester), 1-O-oleyl-2-oleoyl-3-dimethylaminopropyl-p-hydroxyethylammonium (DORI ester/ether), and their salts promote in vivo gene delivery. In some embodiments, cationic lipids comprise groups attached via a heteroatom attached to the quaternary ammonium moiety in the head group. A glycyl spacer can connect the linker to the hydroxyl group.

Specific, but non-limiting cationic lipids for use in certain embodiments of the present invention include DMRIE ((±)-N-(2-hydroxyethyl)-N,N-dimethyl-2,3-bis(tetradecyloxy)-1-propanaminium bromide), GAP-DMORIE ((±)-N-(3-aminopropyl)-N,N-dimethyl-2,3-bis(syn-9-tetradeceneyloxy)-1-propanaminium bromide), and GAP-DLRIE ((±)-N-(3-aminopropyl)-N,N-dimethyl-2,3-(bis-dodecyloxy)-1-propanaminium bromide).

Other cationic lipids include (±)-N,N-dimethyl-N-[2-(sperminecarboxamido)ethyl]-2,3-bis(dioleyloxy)-1-propaniminium pentahydrochloride (DOSPA), (±)-N-(2-aminoethyl)-N,N-dimethyl-2,3-bis(tetradecyloxy)-1-propaniminium bromide (β-aminoethyl-DMRIE or βAE-DMRIE) (Wheeler, et al., Biochim. Biophys. Acta 1280:1-11 (1996)), and (±)-N-(3-aminopropyl)-N,N-dimethyl-2,3-bis(dodecyloxy)-1-propaniminium bromide (GAP-DLRIE) (Wheeler, et al., Proc. Natl. Acad. Sci. USA 93:11454-11459 (1996)), which have been developed from DMRIE.

Other examples of DMRIE-derived cationic lipids that are useful for the present invention are (±)-N-(3-aminopropyl)-N,N-dimethyl-2,3-(bis-decyloxy)-1-propanaminium bromide (GAP-DDRIE), (±)-N-(3-aminopropyl)-N,N-dimethyl-2,3-(bis-tetradecyloxy)-1-propanaminium bromide (GAP-DMRIE), (±)-N-((N″-methyl)-N′-ureyl)propyl-N,N-dimethyl-2,3-bis(tetradecyloxy)-1-propanaminium bromide (GMU-DMRIE), (±)-N-(2-hydroxyethyl)-N,N-dimethyl-2,3-bis(dodecyloxy)-1-propanaminium bromide (DLRIE), and (±)-N-(2-hydroxyethyl)-N,N-dimethyl-2,3-bis-([Z]-9-octadecenyloxy)propyl-1-propaniminium bromide (HP-DORIE).

In the embodiments where the immunogenic composition comprises a cationic lipid, the cationic lipid may be mixed with one or more co-lipids. For purposes of definition, the term “co-lipid” refers to any hydrophobic material which may be combined with the cationic lipid component and includes amphipathic lipids, such as phospholipids, and neutral lipids, such as cholesterol. Cationic lipids and co-lipids may be mixed or combined in a number of ways to produce a variety of non-covalently bonded macroscopic structures, including, for example, liposomes, multilamellar vesicles, unilamellar vesicles, micelles, and simple films. One non-limiting class of co-lipids are the zwitterionic phospholipids, which include the phosphatidylethanolamines and the phosphatidylcholines. Examples of phosphatidylethanolamines, include DOPE, DMPE and DPyPE. In certain embodiments, the co-lipid is DPyPE, which comprises two phytanoyl substituents incorporated into the diacylphosphatidylethanolamine skeleton.

When a composition of the present invention comprises a cationic lipid and co-lipid, the cationic lipid:co-lipid molar ratio may be from about 9:1 to about 1:9, from about 4:1 to about 1:4, from about 2:1 to about 1:2, or about 1:1.

In order to maximize homogenity, the cationic lipid and co-lipid components may be dissolved in a solvent such as chloroform, followed by evaporation of the cationic lipid/co-lipid solution under vacuum to dryness as a film on the inner surface of a glass vessel (e.g., a Rotovap round-bottomed flask). Upon suspension in an aqueous solvent, the amphipathic lipid component molecules self-assemble into homogenous lipid vesicles. These lipid vesicles may subsequently be processed to have a selected mean diameter of uniform size prior to complexing with, for example, a codon-optimized polynucleotide of the present invention, according to methods known to those skilled in the art. For example, the sonication of a lipid solution is described in Felgner et al., Proc. Natl. Acad. Sci. USA 84, 7413-7417 (1987) and in U.S. Pat. No. 5,264,618, the disclosures of Which are incorporated herein by reference.

In those embodiments where the composition includes a cationic lipid, polynucleotides of the present invention are complexed with lipids by mixing, for example, a plasmid in aqueous solution and a solution of cationic lipid:co-lipid as prepared herein are mixed. The concentration of each of the constituent solutions can be adjusted prior to mixing such that the desired final plasmid/cationic lipid:co-lipid ratio and the desired plasmid final concentration will be obtained upon mixing the two solutions. The cationic lipid:co-lipid mixtures are suitably prepared by hydrating a thin film of the mixed lipid materials in an appropriate volume of aqueous solvent by vortex mixing at ambient temperatures for about 1 minute. The thin films are prepared by admixing chloroform solutions of the individual components to afford a desired molar solute ratio followed by aliquoting the desired volume of the solutions into a suitable container. The solvent is removed by evaporation, first with a stream of dry, inert gas (e.g. argon) followed by high vacuum treatment.

Other hydrophobic and amphiphilic additives, such as, for example, sterols, fatty acids, gangliosides, glycolipids, lipopeptides, liposaccharides, neobees, niosomes, prostaglandins and sphingolipids, may also be included in compositions of the present invention. In such compositions, these additives may be included in an amount between about 0.1 mol % and about 99.9 mol % (relative to total lipid), about 1-50 mol %, or about 2-25 mol %.

Additional embodiments of the present invention are drawn to compositions comprising an auxiliary agent. The present invention is further drawn to methods to use such compositions, methods to make such compositions, and pharmaceutical kits. As used herein, an “auxiliary agent” is a substance included in a composition for its ability to enhance, relative to a composition which is identical except for the inclusion of the auxiliary agent, the entry of polynucleotides into vertebrate cells in vivo, and/or the in vivo expression of polypeptides encoded by such polynucleotides. Auxiliary agents of the present invention include nonionic, anionic, cationic, or zwitterionic surfactants or detergents, in particular, nonionic surfactants or detergents, chelators, DNase inhibitors, agents that aggregate or condense nucleic acids, emulsifying or solubilizing agents, wetting agents, gel-forming agents, and buffers.

Auxiliary agents for use in compositions of the present invention include, but are not limited to non-ionic detergents and surfactants IGEPAL CA 630® CA 630, NONIDET NP-40, Nonidet® P40, Tween-20®, Tween-80®, Pluronic® F68, Pluronic F77®, Pluronic P65®, Triton X-100™, and Triton X-114™; the anionic detergent sodium dodecyl sulfate (SDS); the sugar stachyose; the condensing agent DMSO; and the chelator/DNAse inhibitor EDTA. In certain specific embodiments, the auxiliary agent is DMSO, Nonidet P40, Pluronic F68®, Pluronic F77®, Pluronic P65®, Pluronic L64®, and Pluronic F108®. See, e.g., U.S. patent application Publication 20020019358, published Feb. 14, 2002, which is incorporated herein by reference in its entirety.

Compositions of the present invention can be formulated according to known methods. Suitable preparation methods are described, for example, in Remington's Pharmaceutical Sciences, 16^{th} Edition, A. Osol, ed., Mack Publishing Co., Easton, Pa. (1980), and Remington's Pharmaceutical Sciences, 19^{th} Edition, A. R. Gennaro, ed., Mack Publishing Co., Easton, Pa. (1995), both of which are incorporated herein by reference in their entireties. Although the composition may be administered as an aqueous solution, it can also be formulated as an emulsion, gel, solution, suspension, lyophilized form, or any other form known in the art. In addition, the composition may contain pharmaceutically acceptable additives including, for example, diluents, binders, stabilizers, and preservatives.

Certain compositions of the present invention may further include one or more known adjuvants. The term “adjuvant” refers to any material having the ability to (1) alter or increase the immune response to a particular antigen or (2) increase or aid an effect of a pharmacological agent. It should be noted, with respect to polynucleotide vaccines, that an “adjuvant,” may be a transfection facilitating material. Similarly, certain “transfection facilitating materials” described supra, may also be an “adjuvant.” An adjuvant may be used with a composition comprising a polynucleotide of the present invention. In a prime-boost regiment, as described herein, an adjuvant may be used with either the priming immunization, the booster immunization, or both. Suitable adjuvants include, but are not limited to, cytokines and growth factors; bacterial components (e.g., endotoxins, in particular superantigens, exotoxins and cell wall components); aluminum-based salts; calcium-based salts; silica; polynucleotides; toxoids; serum proteins, viruses and virally-derived materials, poisons, venoms, and cationic lipids.

The ability of an adjuvant to increase the immune response to an antigen is typically manifested by a significant increase in immune-mediated protection. For example, an increase in humoral immunity is typically manifested by a significant increase in the titer of antibodies raised to the antigen, and an increase in T-cell activity is typically manifested in increased cell proliferation, or cellular cytotoxicity. An adjuvant may also alter an immune response, for example, by changing a primarily humoral or Th₂ response into a primarily cellular, or Th₁ response.

In certain adjuvant compostions, the adjuvants are cytokines. A composition of the present invention can comprise one or more cytokines, chemokines, or compounds that induce the production of cytokines and chemokines, or a polynucleotide encoding one or more cytokines, chemokines, or compounds that induce the production of cytokines and chemokines. Examples include, but are not limited to granulocyte macrophage colony stimulating factor (GM-CSF), granulocyte colony stimulating factor (G-CSF), macrophage colony stimulating factor (M-CSF), colony stimulating factor (CSF), erythropoietin (EPO), interleukin 2 (IL-2), interleukin-3 (IL-3), interleukin 4 (IL-4), interleukin 5 (IL-5), interleukin 6 (IL-6), interleukin 7 (IL-7), interleukin 8 (IL-8), interleukin 10 (IL-10), interleukin 12 (IL-12), interleukin 15 (IL-15), interleukin 18 (IL-18), interferon alpha (IFNα), interferon beta (IFNβ), interferon gamma (IFNγ), interferon omega (IFNω), interferon tau (IFNτ), interferon gamma inducing factor I (IGIF), transforming growth factor beta (TGF-β), RANTES (regulated upon activation, normal T-cell expressed and presumably secreted), macrophage inflammatory proteins (e.g., MIP-1 alpha and MIP-1 beta), Leishmania elongation initiating factor (LEIF), and Flt-3 ligand.

In certain compositions of the present invention, the polynucleotide construct may be complexed with an adjuvant composition comprising (±)-N-(3-aminopropyl)-N,N-dimethyl-2,3-bis(syn-9-tetradeceneyloxy) 1-propanaminium bromide (GAP-DMORIE). The composition may also comprise one or more co-lipids, e.g., 1,2-dioleoyl-sn-glycero-3-phosphoethanolamine (DOPE), 1,2-diphytanoyl-sn-glycero-3-phosphoethanolamine (DPyPE), and/or 1,2-dimyristoyl-glycer-3-phosphoethanolamine (DMPE). An adjuvant composition comprising; GAP-DMORIE and DPyPE at a 1:1 molar ratio is referred to herein as Vaxfectin™. See, e.g., PCT Publication No. WO 00/57917, which is incorporated herein by reference in its entirety.

Nucleic acid molecules and/or polynucleotides of the present invention, e.g., pDNA, mRNA, linear DNA or oligonucleotides, may be solubilized in any of various buffers. Suitable buffers include, for example, phosphate buffered saline (PBS), normal saline, Tris buffer, and sodium phosphate (e.g., 150 mM sodium phosphate). Insoluble polynucleotides may be solubilized in a weak acid or weak base, and then diluted to the desired volume with a buffer. The pH of the buffer may be adjusted as appropriate. In addition, a pharmaceutically acceptable additive can be used to provide an appropriate osmolarity. Such additives are within the purview of one skilled in the art. For aqueous compositions used in vivo, sterile pyrogen-free water can be used. Such formulations will contain an effective amount of a polynucleotide together with a suitable amount of an aqueous solution in order to prepare pharmaceutically acceptable compositions suitable for administration to a vertebrate.

### EXAMPLES

Materials and Methods

The following materials and methods apply generally to all the examples disclosed herein. Specific materials and methods are disclosed in each example, as necessary.

The practice of the present invention will employ, unless otherwise indicated, conventional techniques of cell biology, cell culture, molecular biology (including PCR), vaccinology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); and in Ausubel et al., Current Protocols in Molecular Biology, John Wiley and Sons, Baltimore, Md. (1989).

Plasmid Vector

Constructs of the present invention were inserted into eukaryotic expression vector V1012. This vector is built on a modified pUC18 background (see Yanisch-Perron, C., et al. Gene 33:103-119 (1985)), and contains a kanamycin resistance gene, the human cytomegalovirus immediate early 1 promoter/enhancer and intron A, and the bovine growth hormone transcription termination signal, and a polylinker for inserting foreign genes. See Hartikka, J., et al., Hum. Gene Ther. 7:1205-1217 (1996). However, other standard commercially available eukaryotic expression vectors may be used in the present invention, including, but not limited to: plasmids pcDNA3, pCMV/Zeo, pCR3.1, pEF1/His, pIND/GS, pRc/CMV2, pSV40/Zeo2, pTRACER-CMV, pUB6/V5-His, pVAX1, and pZeoSV2 (available from Invitrogen, San Diego, Calif.), and plasmid pCI (available from Promega, Madison, Wis.).

Plasmid DNA Purification

Plasmid DNA was transformed into Escherichia coli DH5α competent cells and highly purified covalently closed circular plasmid DNA was isolated by a modified lysis procedure (Horn, N. A., et al., Hum. Gene Ther. 6:565-573 (1995)) followed by standard double CsCl-ethidium bromide gradient ultracentrifugation (Sambrook, J., et al., Molecular Cloning: A Laboratory Manual, 2nd Ed., Cold Spring Harbor Laboratory Press, Plainview, N.Y. (1989)). Alternatively, plasmid DNAs are purified using Giga columns from Qiagen (Valencia, Calif.) according to the kit instructions. All plasmid preparations were free of detectable chromosomal DNA, RNA and protein impurities based on gel analysis and the bicinchoninic protein assay (Pierce Chem. Co., Rockford Ill.). Endotoxin levels were measured using Limulus Amebocyte Lysate assay (LAL, Associates of Cape Cod, Falmouth, Mass.) and were less than 0.6 Endotoxin Units/mg of plasmid DNA. The spectrophotometric A₂₆₀/A₂₈₀ ratios of the DNA solutions were typically above 1.8. Plasmids were ethanol precipitated and resuspended in an appropriate solution, e.g., 150 mM sodium phosphate (for other appropriate excipients and auxiliary agents, see U.S. patent application Publication 20020019358, published Feb. 14, 2002). DNA was stored at −20° C. until use. DNA was diluted by mixing it with 300 mM salt solutions and by adding appropriate amount of USP water to obtain 1 mg/ml plasmid DNA in the desired salt at the desired molar concentration.

Injections of Plasmid DNA

The quadriceps muscles of restrained awake mice (e.g., female 6-12 week old BALB/c mice from Harlan Sprague Dawley, Indianapolis, Ind.) are injected bilaterally with 50 μg of DNA in 50 μl solution (100 μg in 100 μl total per mouse) using a disposable sterile, plastic insulin syringe and 28 G ½ needle (Becton-Dickinson, Franklin Lakes, N.J., Cat. No. 329430) fitted with a plastic collar cut from a micropipette tip, all as previously described (Hartikka, J., et al., Hum. Gene Ther. 7:1205-1217 (1996)).

Animal care throughout the study was in compliance with the “Guide for the Use and Care of Laboratory Animals”, Institute of Laboratory Animal Resources, Commission on Life Sciences, National Research Council, National Academy Press, Washington, D.C., 1996 as well as with Vical's Institutional Animal Care and Use Committee.

Immune Correlates

Since anthrax challenge experiments must be carried under strict containment conditions, they can be difficult and expensive, even in laboratory animals. Accordingly, it has been very important for workers in this area to develop in vitro assays to measure levels of immunity and to demonstrate that these assays sufficiently correlate to in vivo challenges. A number of in vitro assays, which are known to those of ordinary skill in the art to be correlates for challenges have been have been developed. See, e.g., Reuveny, S. et al. Infect. Immun. 69:2888-2893 (2001); Kobiler, D. et al. Infect. Immun. 70:544-560 (2002); Pitt, M. L. et al. Vaccine 19:4768-4773 (2001); and Park, S., and Leppla, S. H. Protein Expr. Purif. 18:293-302 (2000), each of which is incorporated herein by reference in its entirety. An additional assay is described in Example 9(b), infra.

### Example 1

### Construction of an Isolated Polynucleotide Comprising a Human Codon-Optimized PA Coding Region, Encoding the Full Length Bacillus Anthracis Protective Antigen (PA)

A representative native Bacillus anthracis protective antigen (PA) nucleotide sequence consists of nucleotides 1804 to 4098 of GenBank accession number M22589 version M22589.1 GI:143280 (SEQ ID NO:3). See Welkos, S. L. et al. Gene 69:287-300 (1988), which is incorporated herein by reference in its entirety. The PA sequence encodes a 764 amino acid (aa) precursor protein (SEQ ID NO:4) that is processed by a signal peptidase upon secretion by the bacteria, and also by host serum proteases (reviewed in Mesnage S., and Fouet, A. J. Bacteriol. 184:331-334 (2002), which is incorporated by reference herein in its entirety). The first 29 amino acids of PA encodes a bacterial signal sequence that is cleaved during secretion from the bacteria. In the host, furin-like serum proteases cleave off the N-terminal 258 amino acids to yield PA63, the active form of PA that can bind lethal factor (LF) and edema factor (EF), thereby causing toxicity.

A nucleic acid coding region for full-length PA (SEQ ID NO:4), optimized for human codon usage was derived by determining codon frequencies from the human codon usage table (Table 2) as described above. The codon-optimized nucleic acid sequence was created by using the various codons encoding the amino acids of SEQ ID NO:4, each at the frequencies with which they occur in the codon usage table of Table 2. Although any codon-optimized coding region which encodes SEQ ID NO:4 may be used, including, but not limited to SEQ ID Nos 23, 24, or 25, this Example and other Examples below use the human codon-optimized coding region encoding SEQ ID NO:4 represented by SEQ ID NO:23. Alternatively a human codon-optimized nucleic acid coding region encoding SEQ ID NO:4 can be prepared by referring to the codon usage table of Table 2, and using only the most frequent codons for each amino acid, as represented by SEQ ID NO:21.

The nucleic acid represented by SEQ ID NO:23 is constructed in the following manner. First, a series complementary oligonucleotide pairs of 80-90 nucleotides each in length and spanning the length of SEQ ID NO:23 are synthesized by standard methods. These oligonucleotide pairs are synthesized such that upon annealing, they form double stranded fragments of 80-90 base pairs, containing cohesive ends. The single-stranded ends of each pair of oligonucleotides are designed to anneal with a single-stranded end of an adjacent oligonucleotide duplex. Several adjacent oligonucleotide pairs prepared in this manner are allowed to anneal, and approximately five to six adjacent oligonucleotide duplex fragments are then allowed to anneal together via the cohesive single stranded ends. This series of annealed oligonucleotide duplex fragments is then ligated together and cloned into the TOPO® vector available from Invitrogen Corporation, Carlsbad, Calif. The construct is then sequenced by standard methods. Constructs prepared in this manner, comprising 5 to 6 adjacent 80 to 90 base pair fragments ligated together, i.e., fragments of about 500 base pairs, are prepared, such that the entire desired sequence of SEQ ID NO:23 is represented in a series of plasmid constructs. The inserts of these plasmids are then cut with appropriate restriction enzymes and ligated together to form the final construct. The final construct is then cloned into a standard bacterial cloning vector, and sequenced.

### Example 2

### Construction of an Isolated Polynucleotide Comprising a Human Codon-Optimized LF Coding Region, Encoding the Full Length Bacillus Anthracis Lethal Factor (LF)

A representative native Bacillus anthracis lethal factor (LF) nucleotide sequence consists of nucleotides 685 to 3111 of GenBank accession number M30210 version M30210.1 GI:143141 (SEQ ID NO:11). The LF sequence encodes a 809 amino acid precursor protein that is processed to a 775 amino acid secreted protein by cleavage of its signal sequence. LF is a zinc metalloprotease that cleaves mitogen-activated protein kinase kinases (MAPKKs) contained inside target cells. See Mesnage S., and Fouet, A. J. Bacteriol. 184:331-334 (2002). Numerous mutations in LF have been described that eliminate zinc binding or the catalytic site of LF resulting in the loss of toxicity. See Hammond, S. E., and Hanna, P. C. Infect. Immun. 66:2374-2378 (1998). One form of inactive LF is described in detail herein, but all others could also be used with an identical approach.

A nucleic acid coding region for full-length LF (SEQ ID NO:12), optimized for human codon usage was derived by determining codon frequencies from the human codon usage table (Table 2) as described above. The codon-optimized nucleic acid sequence was created by using the various codons encoding the amino acids of SEQ ID NO:12, each at the frequencies with which they occur in the codon usage table of Table 2. Although any codon-optimized coding region which encodes SEQ ID NO:12 may be used, including, but not limited to SEQ ID NOs 26, 27, and 28, this Example and other Examples below use the human codon-optimized coding region encoding SEQ ID NO:12 represented by SEQ ID NO:26. Alternatively a human codon-optimized nucleic acid coding region encoding SEQ ID NO:12 can be prepared by referring to the codon usage table of Table 2, and using only the most frequent codons for each amino acid, as represented by SEQ ID NO:22.

The nucleic acid represented by SEQ ID NO:26 is constructed commercially by Retrogen, San Diego, Calif., in the following manner. First, a series complementary oligonucleotide pairs of 80-90 nucleotides each in length and spanning the length of SEQ ID NO:26 are synthesized by standard methods. These oligonucleotide pairs are synthesized such that upon annealing, they form double stranded fragments of 80-90 base pairs, containing cohesive ends. The single-stranded ends of each pair of oligonucleotides are designed to anneal with a single-stranded end of an adjacent oligonucleotide duplex. Several adjacent oligonucleotide pairs prepared in this manner are allowed to anneal, and approximately five to six adjacent oligonucleotide duplex fragments are then allowed to anneal together via the cohesive single stranded ends. This series of annealed oligonucleotide duplex fragments are then ligated together and cloned into a the TOPO® vector available from Invitrogen Corporation, Carlsbad, Calif. The construct is then sequenced by standard methods. Constructs prepared in this manner, comprising 5 to 6 adjacent 80 to 90 base pair fragments ligated together, i.e., fragments of about 500 base pairs, are prepared, such that the entire desired sequence of SEQ ID NO:26 is represented in a series of plasmid constructs. The inserts of these plasmids are then cut with appropriate restriction enzymes and ligated together to form the final construct. The final construct is then cloned into a standard bacterial cloning vector, and sequenced.

### Example 3

### Construction of Plasmid Constructs Comprising Fragments, Variants, and Derivatives of a Human Codon-Optimized Coding Region Encoding Bacillus Anthracis PA

Several fragments, variants, and derivatives based on SEQ ID NO:23, the human codon-optimized coding region encoding Bacillus anthracis PA described in Example 1, were constructed in the following manner. Codon-optimized nucleic acid fragments encoding three alternate forms of PA were constructed, namely, a nucleic acid fragment encoding full-length PA minus the furin cleavage site (PA83Δ Furin), a nucleic acid fragment encoding the active furin cleavage product of mature PA (PA63), and a nucleic acid fragment encoding the active furin cleavage product of mature PA in which Phe 342 and 343 have been deleted (PA63ΔFF). Each of these nucleic acid fragments were fused in-frame to a nucleic acid encoding a human tissue plasminogen activator (TPA) signal peptide sequence that directs the expressed PA variants and/or fragments to the secretory pathway in mammalian cells. Other useful PA fragments, variants and/or derivatives will be readily apparent to those of ordinary skill in the art, and are included in the present invention.

a) Construction of TPA-PA63.

PA63, the C-terminal fragment of PA corresponding to amino acids 199-764 of SEQ ID NO:4 corresponds to the mature, extracellularly processed protein that is able to bind to LF and edema factor (EF).

TPA-PA63 (FIG. 1, SEQ ID NO:1) was constructed commercially by Retrogen, San Diego, Calif. A large number of other companies which provide similar construction of predetermined nucleic acid sequences are well known to those of ordinary skill in the art. The sequence was constructed in the following manner. First, a series complementary oligonucleotide pairs of 80-90 nucleotides each in length and spanning the length of SEQ ID NO:1 were synthesized by standard methods. These oligonucleotide pairs were synthesized such that upon annealing, they formed double stranded fragments of 80-90 base pairs, containing cohesive ends. The single-stranded ends of each pair of oligonucleotides were designed to anneal with a single-stranded end of an adjacent oligonucleotide duplex. Several adjacent oligonucleotide pairs prepared in this manner were allowed to anneal, and approximately five to six adjacent oligonucleotide duplex fragments were then allowed to anneal together via the cohesive single stranded ends. This series of annealed oligonucleotide duplex fragments were then ligated together and cloned into a the TOPO® vector available from Invitrogen Corporation, Carlsbad, Calif. The construct was then sequenced by standard methods. Constructs prepared in this manner, comprising 5 to 6 adjacent 80 to 90 base pair fragments ligated together, i.e., fragments of about 500 base pairs, were prepared, such that the entire desired sequence of SEQ ID NO:1 was represented in a series of plasmid constructs. The inserts of these plasmids were then cut with appropriate restriction enzymes and ligated together in the TOPO® vector.

This construct was cut with EcoRV+BamHI and the 1788 bp insert fragment (i.e., SEQ ID NO:1) was cloned into the same sites of the VR1012 expression plasmid (see Hartikka et al., Hum. Gene Therapy 7:1205-1217 (1996), which is incorporated herein by reference in its entirety). The resulting plasmid, designated VR6290, was sequenced and expressed in transiently transfected VM-92 cells in culture (see Example 6) to confirm the expression and secretion of the construct.

b) Construction of TPA-PA63ΔFF.

A different non-toxic form of PA can be generated by deleting the two phenylalanine residues at positions 342 and 343 of SEQ ID NO:4 to produce a PA protein that cannot heptamerize and form a pore to allow LF and EF to enter the cytoplasm of an infected cell. See, e.g., Singh, Y. et al. J. Biol. Chem. 269:29039-29046 (1994), which is incorporated herein by reference in its entirety.

An expression plasmid comprising TPA-PA63Δ F (FIG. 2, SEQ ID NO:5) was prepared by the following method. Plasmid VR6290, prepared as described in section (a), supra, was used as a template for PCR with the following two sets of PCR primers using Turbo Pfu polymerase from Stratagene Inc., La Jolla, Calif.

(SEQ ID NO:29)

1. TPA-for 5′GAGCTTGATA TCGCCACCAT GGATGC 3′
and

(SEQ ID NO:30)

PA del FF-Rev 5′CCACCAATAT CCGATGCATG GACTTCCGC 3′
produced a 520 bp fragment.

(SEQ ID NO:31)

2. HPA-end Rev 5′CTTGAAGGAT CCTCAACCGA TCTCGTAG 3′
and

(SEQ ID NO:32)

PA del FF-For 5′CCATGCATCG GATATTGGTG GCTCCGTGTC
3′ produced a 1280 bp fragment that overlapped
fragment 1.

Fragments 1 and 2 were gel purified using the QIAquick Gel Extraction Kit from Qiagen Inc (Valencia, Calif.) and the fragments were combined in a subsequent PCR reaction and amplified with the primer pair TPA-for and HPA-endRev to yield the full length 1782 bp fragment shown in FIG. 2. This fragment was digested with the restriction enzymes EcoR5+BamHI and ligated into the same sites of the VR1012 expression plasmid. The resulting plasmid, designated VR6291, was sequenced and expressed in transiently transfected VM-92 cells in culture (see Example 6) to confirm the expression and secretion of the construct.

c) Construction of TPA-PA83Δ Furin.

Deleting the furin cleavage site of the mature PA, i.e., amino acids 192-197 (Ser-Arg-Lys-Lys-Arg-Ser) of SEQ ID NO:4, yields a protein that is secreted from the cell and that can bind the host cell receptor but cannot bind LF or EF and therefore is non-toxic. See, e.g., Singh, Y. et al. Infect. Immun. 66:3447-3448 (1998), and Klimpel K R, et al. Proc. Natl. Acad. Sci. USA 89:10277-10281 (1992), which are incorporated herein by reference in their entireties.

An expression plasmid comprising TPA-PA83Δfurin (FIG. 3, SEQ ID NO:7) was constructed in the following manner. A plasmid comprising a codon-optimized nucleotide sequence (as per the sequence of SEQ ID NO:23) encoding the N-terminal 20 kD domain of PA, i.e., corresponding to the portion of PA that is cleaved off by furin, was synthesized by Retrogen Inc. according to the method described in section a), supra. This plasmid was cut with EcoRV+AfeI and the 570 bp insert was gel purified as above. The plasmid VR6290 described in section a) above was digested with EcoRV+AfeI and the 6.6 kb linear vector fragment was gel purified and ligated to the 570 bp N-terminal fragment. Transformed colonies were screened for recombinants by PCR using the primer pair NtermPA seqF 5′ GTGGACGACC AGGAAGTGAT C 3′ (SEQ ID NO:33) and NtermPA seqR 5′ GGCTATCTGT CCAGTACAGC TTGAA3′ (SEQ ID NO:34). A selected recombinant, designated VR6292, was sequenced and was expressed in transiently transfected VM-92 cells in culture (see Example 6) to confirm the expression and secretion of the construct.

### Example 4

### Construction of Plasmid Constructs Comprising Fragments, Variants, and Derivatives of a Human Codon-Optimized Coding Region Encoding Bacillus Anthracis LF

Several fragments, variants, and derivatives of SEQ ID NO:26, the human codon-optimized coding region encoding Bacillus anthracis LF, as prepared in Example 2, were constructed in the following manner. Codon-optimized nucleic acid fragments encoding four alternate forms of LF were constructed, namely, a nucleic acid fragment encoding the full-length mature LF in which His 686, His 690 and Glu 687 have been substituted with Ala, Ala, and Asp, respectively (LF HEXXH), a nucleic acid fragment encoding amino acids 34 to 583 of full-length LF, encoding domains I-III of mature LF (LF Domain I-III), a nucleic acid fragment encoding amino acids 34 to 254 of mature LF, corresponding to domain I of mature LF (LF Domain IA), and a nucleic acid fragment encoding amino acids 34 to 295 of mature LF, corresponding to domain I of mature LF (LF Domain IB). Each of these nucleic acid fragments were fused in-frame to a nucleic acid encoding a human tissue plasminogen activator (TPA) signal peptide sequence that directs the expressed LF variants and/or fragments to the secretory pathway in mammalian cells. Furthermore, other useful LF fragments, variants and/or derivatives would be readily apparent to those of ordinary skill in the art.

a) Construction of TPA-LF HEXXH.

This construct encodes full length LF (minus the bacterial signal sequence) with three point mutations that render LF non-toxic. Each of these mutations, alone or together, are thought to eliminate the enzymatic activity of LF, thereby rendering it non-toxic. See, e.g., Hammond, S E and Hanna P C, Infect. Immun. 66:2374-2378 (1998), which is incorporated herein by reference in its entirety. Other LF mutants contained in this reference, e.g., LF^{E687C}, LF^{E687D}, LF^{H686A}, LF^{H690A}, and LF^{H686A+H690A}, are also included in the present invention. While not being bound by theory, substitution of the histidine residues at positions 686 and 690 is thought to decrease zinc binding, resulting in decreased or no protease activity, and substitution of the glutamic acid at position 687 is thought to also eliminate protease activity, thereby resulting in no in vitro or in vivo macrophage killing. This construct combines all three mutations to afford a greater perceived level of safety than either point mutation alone.

An expression plasmid comprising LF HEXXH (FIG. 4, SEQ ID NO:9) was prepared in the following manner. The entire 2418 bp sequence was synthesized by Retrogen Inc. and inserted into the EcoRV and BamHI sites of the TOPO vector as described in Example 3(a). The resulting plasmid was digested with EcoRV and BamHI and the 2418 bp insert was purified by gel electrophoresis as described above. The insert was ligated into EcoRV+BamHI digested VR1012 and transformed into E. coli. Transformed colonies were screened for recombinants by PCR using the primer pair seqF1-hLF 5′ CCGTGCTCGT TATTCAGAGT 3′ (SEQ ID NO:35) and seqR2-hLF 5′ CCTTCTCTTC TGTGCTAAGG 3′ (SEQ ID NO:36). A selected recombinant, designated VR6295, was sequenced and was expressed in transiently transfected VM-92 cells in culture (see Example 6) to confirm the expression and secretion of the construct.

b) Construction of TPA-LF Domain I-III.

This construct encodes the N-terminal amino acids 34-583 of mature LF, corresponding to domains I-III. The entire protease domain (domain IV) has been deleted and is therefore non-toxic. See, e.g., Pannifer A D et al. Nature 414:229-233 (2001), which is incorporated herein by reference in its entirety.

Recent data suggest LF is capable of entering cells independently of PA using a region at the N terminal domain (Kushner, N. et al. Proc. Natl. Acad. Sci. 100: 6652-7 (2003)). In addition, the full length LF is able to cause impairment of dendritic cell function via domain IV protease degredation of MAP activated protein kinase kinase (MAPKK). Agrawal, A. et al. Nature 424: 329-34 (2003)). Therefore LF, independently of its association with PA, may have toxic effects which could be blocked through vaccination. Through the inclusion of an LF component in the vaccine of the present invention, it may be possible to neutralize LF at a number of domains and to block potential toxicities that occur in conjunction with, or independent of, binding to PA. In may also be possible to block the primary binding of LF to PA.

An expression plasmid comprising TPA-LF Domain I-III (FIG. 5, SEQ ID NO:13) was prepared in the following manner. The plasmid VR6295 (as produced in section a) above, was PCR amplified with the primer pair TPA-for (SEQ ID NO:29) and LF-DomII-R 5′ GAACCTGGAT CCCTACACCA CCTTGGCGTC GATG 3′ (SEQ ID NO:37) using Pfu polymerase. The 1740 bp fragment was gel purified, digested with EcoRV+BamHI and cloned into VR1012. Transformed colonies were screened by PCR using the same amplification primers. A selected recombinant, designated VR62952, was sequenced and was expressed in transiently transfected VM-92 cells in culture (see Example 6) to confirm the expression and secretion of the construct.

c) Construction of TPA-LF Domain IA.

This construct encodes the N-terminal amino acids 34-254 of mature LF, corresponding generally to domain I. This is the portion of LF that directly binds PA. See, e.g., Pannifer A D et al. Nature 414:229-233 (2001).

An expression plasmid comprising TPA-LF Domain I (FIG. 6, SEQ ID NO:15) was prepared in the following manner. The plasmid VR6295 (as produced in section a) above, was PCR amplified with the primer pair TPA-for (SEQ ID NO:29) and G-LF-R 5′GCTAATGGAT CCTCAAAATG CCTTGGCGAA CACCT 3′ (SEQ ID NO:38) using Pfu polymerase. The 753 bp fragment was gel purified, digested with EcoRV+BamHI and cloned into VR1012. Transformed colonies were screened by PCR using the same amplification primers. A selected recombinant, designated VR6295G, was sequenced and was expressed in transiently transfected VM-92 cells in culture (see Example 6) to confirm the expression and secretion of the construct.

d) Construction of TPA-LF Domain IB.

This construct encodes the N-terminal amino acids 34-295 of mature LF, also corresponding generally to domain I.

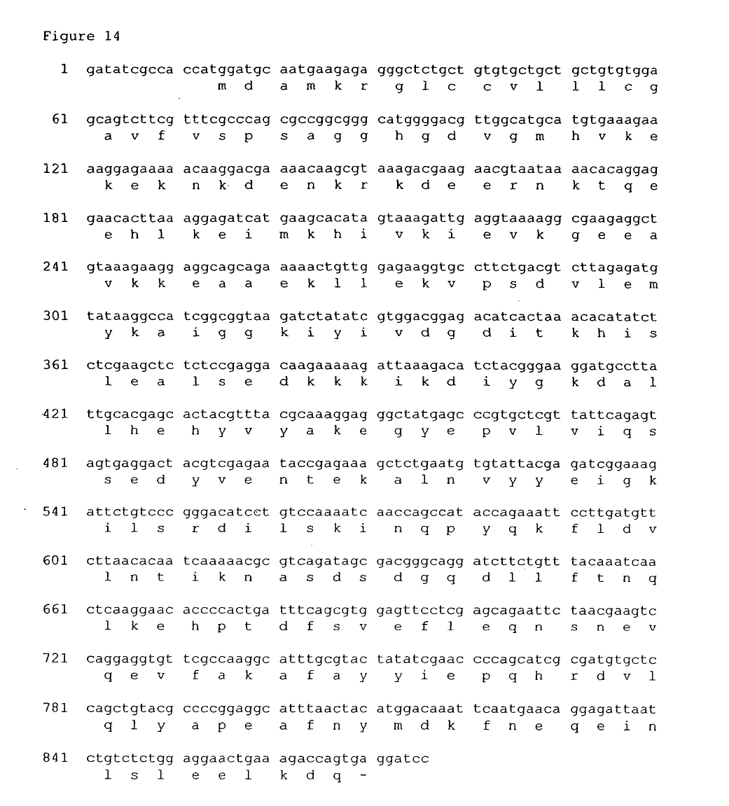

An expression plasmid comprising TPA-LF Domain IB (FIG. 14, SEQ ID NO:39) was prepared in the following manner. The plasmid VR6295 (as produced in section a) above, was PCR amplified with the primer pair TPA-for (SEQ ID NO:29) and crystal-LF-R 5′ CCATACGGAT CCTCACTGGT CTTTCAGTTC CTCCA 3′ (SEQ ID NO:41) using Pfu polymerase. The 876 bp fragment was gel purified, digested with EcoRV+BamHI and cloned into VR1012. Transformed colonies were screened by PCR using the same amplification primers. A selected recombinant, designated VR62951, was sequenced and was expressed in transiently transfected VM-92 cells in culture (see Example 6) to confirm the expression and secretion of the construct.

### Example 5

### N-Linked Glycosylation Mutants

Most mammalian transmembrane and secreted proteins are glycosylated post-translationally in the endoplasmic reticulum. See, e.g., Lodish H et al. Molecular Cell Biology 4^{th} edition, W. H. Freeman and Company, New York. There are two main types of protein glycosylation in mammalian cells, N-linked and O-linked. N-linked glycosylation occurs on aspargine (N) residues at the amino acid motif N-X-(S/T) where X refers to any amino acid residue and S/T denotes serine or threonine. There are seven N-linked glycosylation motifs in mature LF, twelve N-linked glycosylation motifs in full-length mature PA (PA83) and ten N-linked glycosylation motifs in PA63. Since this glycosylation does not occur in bacteria, the anthrax antigens synthesized in mammalian cells after DNA immunization may differ from the PA and LF in anthrax toxin secreted by B. anthracis. See, e.g., Schaffer C. et al. Proteomics 1:248-246 (2001), which is incorporated herein by reference in its entirety. This mammalian N-linked glycosylation could obscure or alter B-cell antibody epitopes that are normally exposed in conventional anthrax protein vaccines. Therefore codon-optimized coding regions encoding PA63 and LF were made in which the asparagines in the N-linked glycosylation motifs (N-X-S/T) motifs were mutated to glutamines (Q-X-S/T). The motif Q-X-S/T is not subject to glycosylation in mammals. Such “sugar minus” variants of any of the variants, fragments, derivatives, or full length coding regions disclosed herein, as well as addition variants, fragments and derivatives known to those of skill in the art are encompassed by the present invention.

All asparagine (N) residues in the N-X-S/T motifs contained in TPA-PA63 (SEQ ID NO:1, produced as described in Example 3(a)) and TPA-LFΔHEXXH (SEQ ID NO:9, produced as described in Example 4(a)) were mutated to Glutamine (Q) by generating a series of overlapping PCR fragments. As described in more detail below, these fragments were added together two at a time and amplified with primers at the extreme end of the two fragments to build larger and larger PCR fragments until a full length mutant was obtained. In each case, the full-length fragment was gel purified, digested with EcoRV+BamHI and cloned into VR1012. All PCR reactions were performed with Pfu polymerase from Stratagene Inc using standard conditions.

a) Construction of TPA-Sugar Minus PA63.

This construct is the same as SEQ ID NO:1, except that all ten N-X-S/T motifs in the encoded polypeptide have been changed to Q-X-S/T, via point mutations. The mutated construct was assembled from overlapping PCR fragments using SEQ ID NO:1 as the template.

Ten nanograms quantities of plasmid VR6290 DNA was amplified with each of the 10 primer pairs listed in Table 8. The fourth column lists the size of the resulting PCR products with the various primer pairs. Each of these resulting PCR fragments has a single stranded region at each end, which can anneal with a single stranded region on another of the fragments.

TABLE 8

| PCR | | | |
|---|---|---|---|
| Frag- | | | |
| ment | Forward Primer | Reverse Primer | Size |
| 1 | TPA-For (SEQ ID NO: 29) | PA-R1 (SEQ ID NO: 42) | 310 bp |
| 2 | PA-F2 (SEQ ID NO: 43) | PA-R2 (SEQ ID NO: 44) | 140 bp |
| 3 | PA-F3 (SEQ ID NO: 45) | PA-R3 (SEQ ID NO: 46) | 90 bp |
| 4 | PA-F4 (SEQ ID NO: 47) | PA-R4 (SEQ ID NO: 48) | 180 bp |
| 5 | PA-F5 (SEQ ID NO: 49) | PA-R5 (SEQ ID NO: 50) | 260 bp |
| 6 | PA-F6 (SEQ ID NO: 51) | PA-R6 (SEQ ID NO: 52) | 100 bp |
| 7 | PA-F7 (SEQ ID NO: 53) | PA-R7 (SEQ ID NO: 54) | 185 bp |
| 8 | PA-F8 (SEQ ID NO: 55) | PA-R8 (SEQ ID NO: 56) | 150 bp |
| 9 | PA-F9 (SEQ ID NO: 57) | PA-R9 (SEQ ID NO: 58) | 130 bp |
| 10 | PA-F10 (SEQ ID NO: 59) | PA-R10 (SEQ ID NO: | 135 bp |
| | | 60) | |
| 11 | PA-F11 (SEQ ID NO: 61) | HPA-endRev (SEQ ID NO: | 80 bp |
| | | 31) | |

2.5 microliters of each PCR fragment in Table 8 was combined pair wise with a second PCR fragment in Table 8. The two fragments were allowed to anneal and were used as templates in a second series of PCR reactions, with resulting PCR fragments as shown in Table 9.

TABLE 9

| PCR | Template | | | |
|---|---|---|---|---|
| Fragment | Fragments | Forward Primer | Reverse Primer | Size |
| 12 | 2 + 3 | PA-F2 | PA-R3 | 230 bp |
| | | (SEQ ID NO: 43) | (SEQ ID NO: 46) | |
| 13 | 4 + 5 | PA-F4 | PA-R5 | 440 bp |
| | | (SEQ ID NO: 47) | (SEQ ID NO: 50) | |
| 14 | 6 + 7 | PA-F6 | PA-R7 | 285 bp |
| | | (SEQ ID NO: 51) | (SEQ ID NO: 54) | |
| 15 | 8 + 9 | PA-F8 | PA-R9 | 280 bp |
| | | (SEQ ID NO: 55) | (SEQ ID NO: 58) | |
| 16 | 10 + 11 | PA-F10 | HPA-endRev | 215 bp |
| | | (SEQ ID NO: 59) | (SEQ ID NO: 31) | |

2.5 microliters of each PCR fragment in Table 9 was combined pair wise with a second PCR fragment in Table 9. The two fragments were allowed to anneal and were used as templates in a third series of PCR reactions, with resulting PCR fragments as shown in Table 10.

TABLE 10

| PCR | Template | | | |
|---|---|---|---|---|
| Fragment | Fragments | Forward Primer | Reverse Primer | Size |
| 17 | 1 + 12 | TPA-For | PA-R3 | 540 bp |
| | | (SEQ ID NO: 29) | (SEQ ID NO: 46) | |
| 18 | 13 + 14 | PA-F4 | PA-R7 | 725 bp |
| | | (SEQ ID NO: 47) | (SEQ ID NO: 54) | |
| 19 | 15 + 16 | PA-F8 | HPA-endRev | 495 bp |
| | | (SEQ ID NO: 55) | (SEQ ID NO: 31) | |

Fragments 17, 18, and 19 were gel purified before proceeding to the next series of PCR reactions. The last two sets of PCR reactions were carried out as listed in Table 11, using 2.5 microliters of the annealed PCR fragment pairs listed in the second column, which had been gel purified.

TABLE 11

| PCR | Template | | | |
|---|---|---|---|---|
| Fragment | Fragments | Forward Primer | Reverse Primer | Size |
| 20 | 17 + 18 | TPA-For | PA-R7 | 1265 bp |
| | | (SEQ ID NO: 29) | (SEQ ID NO: 54) | |
| 21 | 19 + 20 | TPA-For | HPA-endRev | 1788 bp |
| | | (SEQ ID NO: 29) | (SEQ ID NO: 31) | |

Resulting PCR fragment 21 represents the full-length TPA-Sugar minus PA63 fragment (FIG. 7, SEQ ID NO:17). The TPA-sugar minus PA63 fragment was cloned into the VR1012 expression plasmid. A selected recombinant, designated VR6299, was sequenced, and was expressed in transiently transfected VM-92 cells in culture (see Example 6) to confirm the expression and secretion of the construct.

The sequences of the primers used in the PCR reactions in this Example are listed in Table 12.

TABLE 12

| SEQ | | |
|---|---|---|
| ID NO: | Primer | Sequence |
| 29 | TPA-for | GAGCTTGATATCGCCACCATGGATGC |
| 42 | PA-R1 | CTGGAGACACCTGTTTATCGATCC |
| 43 | PA-F2 | GGATCGATAAACAGGTGTCTCCAG |
| 44 | PA-R2 | GAAGTACTGGTCTGTTTAGATATGGT |
| 45 | PA-F3 | ACCATATCTAAACAGACCAGTACTTC |
| 46 | PA-R3 | CGTCGAGGACTGGCTATTGCTAA |
| 47 | PA-F4 | TTAGCAATAGCCAGTCCTCGACG |
| 48 | PA-R4 | GAGGGTCTGCTGTTTGCCCAGG |
| 49 | PA-F5 | CCTGGGCAAACAGCAGACCCTC |
| 50 | PA-R5 | CTTCAGACCACTGTGACCCAGTG |
| 51 | PA-F6 | CACTGGGTCACAGTGGTCTGAAG |
| 52 | PA-R6 | GATCACTGGGCTGCACGGCGG |
| 53 | PA-F7 | CCGCCGTGCAGCCCAGTGATC |
| 54 | PA-R7 | TATTGGTGGCCTGCAGCTCTGC |
| 55 | PA-F8 | GCAGAGCTGCAGGCCACCAATA |
| 56 | PA-R8 | CAGTACTGCTCTGGATAACTTCCC |
| 57 | PA-F9 | GGGAAGTTATCCAGAGCAGTACTG |
| 58 | PA-R9 | AAGCTGGAAATCTGCAGCATATCAT |
| 59 | PA-F10 | ATGATATGCTGCAGATTTCCAGCTT |
| 60 | PA-R10 | CTCGCTTGGCTGGATGATTGTGT |
| 61 | PA-F11 | ACACAATCATCCAGCCAAGCGAG |
| 31 | HPA-endRev | CTTGAAGGATCCTCAACCGATCTCGTAG |

b) Construction of TPA-Sugar Minus LF HEXXH.

This construct is the same as SEQ ID NO:9, except that all seven N-X-S/T motifs in the encoded polypeptide have been changed to Q-X-S/T, via point mutations. The mutated construct was assembled from overlapping PCR fragments using standard methods, using primers which code for Q residues instead of N residues in the seven glycosylation motifs, and using SEQ ID NO:9 as the template.

Ten nanograms quantities of plasmid VR6295 DNA was amplified with each of the 8 primer pairs listed in Table 13. The fourth column lists the size of the resulting PCR products with the various primer pairs. Each of these resulting PCR fragments has a single stranded region at each end, which can anneal with a single stranded region on another of the fragments.

TABLE 13

| PCR | | | |
|---|---|---|---|
| Frag- | | | |
| ment | Forward Primer | Reverse Primer | Size |
| 1 | TPA-For (SEQ ID NO: 29) | LF-R1 (SEQ ID NO: 62) | 170 bp |
| 2 | LF-F2 (SEQ ID NO: 63) | LF-R2 (SEQ ID NO: 64) | 450 bp |
| 3 | LF-F3 (SEQ ID NO: 65) | LF-R3 (SEQ ID NO: 66) | 220 bp |
| 4 | LF-F4 (SEQ ID NO: 67) | LF-R4 (SEQ ID NO: 68) | 580 bp |
| 5 | LF-F5 (SEQ ID NO: 69) | LF-R5 (SEQ ID NO: 70) | 700 bp |
| 6 | LF-F6 (SEQ ID NO: 71) | LF-R6 (SEQ ID NO: 72) | 70 bp |
| 7 | LF-F7 (SEQ ID NO: 73) | LF-R7 (SEQ ID NO: 74) | 65 bp |
| 8 | LF-F8 (SEQ ID NO: 75) | HLFend-R (SEQ ID NO: | 165 bp |
| | | 76) | |

2.5 microliters of each PCR fragment in Table 13 was combined pair wise with a second PCR fragment in Table 13. The two fragments were allowed to anneal and were used as templates in a second series of PCR reactions, with resulting PCR fragments as shown in Table 14.

TABLE 14

| PCR | Template | | | |
|---|---|---|---|---|
| Fragment | Fragments | Forward Primer | Reverse Primer | Size |
| 9 | 1 + 2 | TPA-For | LF-R2 | 620 bp |
| | | (SEQ ID NO: 29) | (SEQ ID NO: 64) | |
| 10 | 3 + 4 | LF-F3 | LF-R4 | 800 bp |
| | | (SEQ ID NO: 65) | (SEQ ID NO: 68) | |
| 11 | 5 + 6 | LF-F5 | LF-R6 | 770 bp |
| | | (SEQ ID NO: 69) | (SEQ ID NO: 72) | |
| 12 | 7 + 8 | LF-F7 | HLFend-R | 230 bp |
| | | (SEQ ID NO: 73) | (SEQ ID NO: 76) | |

2.5 microliters of each PCR fragment in Table 14 was combined pair wise with a second PCR fragment in Table 14. The two fragments were allowed to anneal and were used as templates in a third series of PCR reactions, with resulting PCR fragments as shown in Table 15.

TABLE 15

| PCR | Template | | | |
|---|---|---|---|---|
| Fragment | Fragments | Forward Primer | Reverse Primer | Size |
| 13 | 9 + 10 | TPA-For | LF-R4 | 1420 bp |
| | | (SEQ ID NO: 29) | (SEQ ID NO: 68) | |
| 14 | 11 + 12 | LF-F5 | HLFend-R | 1000 bp |
| | | (SEQ ID NO: 69) | (SEQ ID NO: 76) | |

Fragments 13 and 14 were gel purified before proceeding to the final PCR reaction. This PCR reaction was carried out as listed in Table 16, using 2.5 microliters of the annealed PCR fragment pairs listed in the second column, which had been gel purified.

TABLE 16

| PCR | Template | | | |
|---|---|---|---|---|
| Fragment | Fragments | Forward Primer | Reverse Primer | Size |
| 15 | 13 + 14 | TPA-For | HLFend-R | 2418 bp |
| | | (SEQ ID NO: 29) | (SEQ ID NO: 76) | |

Resulting PCR fragment 15 represents the full-length TPA-Sugar minus LF HEXXH fragment (FIG. 8, SEQ ID NO:19). The TPA-sugar minus LF HEXXH fragment was cloned into the VR1012 expression plasmid. A selected recombinant, designated VR6300, was sequenced and was expressed in transiently transfected VM-92 cells in culture (see Example 6) to confirm the expression and secretion of the construct.

The sequences of the primers used in the PCR reactions in this Example are listed in Table 17.

TABLE 17

| | | |
|---|---|---|
| SEQ ID NO: | Primer | Sequence |

| | | |
|---|---|---|
| 29 | TPA-for | GAGCTTGATATCGCCACCATGGATGC |
| 62 | LF-R1 | TCCTGTGTTTTCTGACGTTCTTCG |
| 63 | LF-F2 | CGAAGAACGTCAGAAAACACAGGA |
| 64 | LF-R2 | TATCTGACGCCTGTTTGATTGTGTT |
| 65 | LF-F3 | AACACAATCAAACAGGCGTCAGATA |
| 66 | LF-R3 | CCAGAGACAGCTGAATCTCCTGTT |
| 67 | LF-F4 | AACAGGAGATTCAGCTGTCTCTGG |
| 68 | LF-R4 | AGCGGTGAGCTGGTTAATATTCATG |
| 69 | LF-F5 | CATGAATATTAACCAGCTCACCGCT |
| 70 | LF-R5 | CCTCAGAATCCTGTCGAAGCTCA |
| 71 | LF-F6 | TGAGCTTCGACAGGATTCTGAGG |
| 72 | LF-R6 | GATCAGACTGCTGCTTATCCAACA |
| 73 | LF-F7 | TGTTGGATAAGCAGCAGTCTGATC |
| 74 | LF-R7 | AGGAAGTCAGCTGACTCCCTTCC |
| 75 | LF-F8 | GGAAGGGAGTCAGCTGACTTCCT |
| 76 | HLFend-R | GCAGATCTGGATCCTCAAGAG |

### Example 6

### In Vitro Expression of Human Codon-Optimized Coding Regions Encoding B. Anthracis PA and LF, and Fragments, Variants and Derivatives Thereof, in a Murine Cell Line

The expression plasmids described Examples 3-5 above and the corresponding wild type Bacillus anthracis genes were initially analyzed in in vitro transiently transfected cells in culture. Initial studies were carried out in a well characterized mouse melanoma cell line (VM-92, also known as UM-449), using cationic lipid-based transfection procedures well known to those of skill in the art. Other standard cell lines, for example, COS-1 cells, COS-7 cells, CHO cells, HEK-293 cells, and HeLa cells, may be used for transient transfections as well. Following transfection, cell lysates and culture supernatants of transfected cells were evaluated to compare relative levels of expression of B. anthracis antigen proteins. The samples were assayed by western blots and ELISAs, using commercially available anti-PA and Anti-LF monoclonal antibodies (available from Research Diagnostics Inc., Flanders N.J.), so as to compare both the quality and the quantity of expressed antigen. Additionally, in vitro transfection assays were used to determine the effect of mixing the various plasmids comprising codon-optimized coding regions encoding non-toxic PA and LF on levels of expression in mammalian cells.

Expression products derived from cells transfected with the various polynucleotide constructs are examined to ensure the correct or predicted molecular weight of the recombinant antigens, and immunoreactivity of the recombinant antigens (i.e., to react with B. anthracis antisera). In addition, a comparison of expression levels (both intra- and extra-cellular) of each class of expression plasmid (e.g., wild type vs. human codon-optimized; truncated vs. full-length) is made.

### Example 7

### In Vitro Expression of Human Codon-Optimized Coding Regions Encoding B. Anthracis PA and LF, and Fragments, Variants and Derivatives Thereof, in a Human Cell Line

The expression plasmids described Examples 3-5 above and the corresponding wild type Bacillus anthracis genes are also analyzed in in vitro transfected human cells in culture. These studies are carried out in a well characterized human cell line, e.g., HeLa cells, ATCC Accession No. CCL-2, available from the American Type Culture Collection, Manassas, Va., using cationic lipid-based transfection procedures well known to those of skill in the art. Following transfection, cell lysates and culture supernatants of transfected cells are evaluated to compare relative levels of expression of B. anthracis antigen proteins. The samples are assayed by western blots and ELISAs, using commercially available antiPA and Anti-LF monoclonal antibodies (available from Research Diagnostics Inc., Flanders N.J.), so as to compare both the quality and the quantity of expressed antigen. Additionally, in vitro transfection assays are used to determine the effect of mixing the various plasmids comprising codon-optimized coding regions encoding non-toxic PA and LF on levels of expression in human cells.

Expression products from the derived from human cells transfected with the various polynucleotide constructs are examined for molecular weight, and expression immunoreactive antigens (i.e., to react with B. anthracis antisera). In addition, a comparison of expression levels (both intra- and extra-cellular) of each class of expression plasmid (e.g., wild type vs. human codon-optimized; truncated vs. full-length) is made.

### Example 8

### Animal Immunization and Challenge

The immunogenicity of expression products encoded by the codon-optimized polynucleotides described in Examples 1-5 are evaluated based on each plasmid's ability to mount a humoral immune response in vivo. Plasmids are tested individually and in combinations by injecting single constructs as well as multiple constructs in various animals as described below. Immunizations are initially carried out in mice by intramuscular (IM) injections. Serum is collected from immunized animals, and the immune response is quantitated by ELISA assay using commercially available antiPA and Anti-LF monoclonal antibodies (available from Research Diagnostics Inc., Flanders N.J.) according to standard protocols. The tests of immunogenicity further include measuring antibody titer, neutralizing antibody titer, and challenging immunized animals with toxin protein.

Testing in rabbits are then used to confirm the results in mice and thereby provide efficacy data for the best plasmids in more than one mammalian immunogenicity model system. Serum is collected from immunized rabbits, and antibody titers and neutralizing antibody titers are determined. In addition, immunized rabbits are tested with a spore inhalation challenge. The combined results determine the plasmids to be subsequently tested in non-human primates.

a) Mouse Immunizations.

The plasmid constructs described in Examples 3-5, as well as similar plasmid constructs comprising native coding regions encoding native PA and LF, as well as empty control plasmids, are tested in vivo in mice by intramuscular injection of the rectus femoris muscle within the quadriceps, using methods described above. There are 5-10 animals per group. A standard DNA vaccination protocol is used (50 μg DNA in 150 mM sodium phosphate (1 mg/ml)/leg at 0, 14 and 28 days). Alternative DNA formulations include PBS instead of sodium phosphate, adjuvants, e.g., Vaxfectin™ at a 4:1 DNA:Vaxfectin™ mass ratio, mono-phosphoryl lipid A (detoxified endotoxin) from S. minnesota (MPL) and trehalosedicorynomycolateAF (TDM), in 2% oil (squalene)-Tween 80-water (MPL+TDM, available from Sigma/Aldrich, St. Louis, Mo., (catalog #M6536)), a solubilized mono-phosphoryl lipid A formulation (AF, available from Corixa), (±)-N-(3-Acetoxypropyl)-N,N-dimethyl-2,3-bis(octyloxy)-1-propanaminium chloride (compound #VC1240), or poloxamers, e.g., CRL1005 (from Organichem) and a solution of benzyl-alkonium chloride “BAK” (from Ruger Chemicals) (“CRL 1005/BAK”) (see Shriver, J. W. et al., Nature 415:331-335 (2002), and P.C.T. Publication No. WO 02/00844 A2, each of which is incorporated herein by reference in its entirety); or transfection-facilitating cationic lipids, e.g., DMRIE/DOPE at a 4:1 DNA:lipid mass ratio.

Serum samples for antibody assays are taken at 0, 21, and 41 days. On or about day 42, the vaccinated animals are challenged using either a tail vein injection of purified lethal factor toxin (Letx) or pulmonary delivery of aerosolized B. anthracis. Mice are challenged using the purified B. anthracis lethal toxin (Letx), i.e., the combined mature PA65 and LF proteins. These proteins are provided through a collaborative agreement with Dr. Stephen Leppla, National Institutes of Dental Research, at the NIH. The proteins are expressed in E. coli as recombinant proteins and purified according to published protocols (see, e.g., Leppla, S H Methods Enzymol. 165:103-116 (1988) and Park, S and Leppla, S H Protein Expr. Purif. 18:293-302 (2000), each of which are incorporated herein by reference in their entireties). The challenge is conducted by injecting the mouse tail vein with a protein cocktail containing 60 μg of purified PA and 25-30 μg of purified LF. This approximates the equivalent of five 50% lethal doses of Letx. The animals are monitored for morbidity and mortality at regular intervals following challenge.

b) Rabbit Immunizations.

The rabbit has increasingly gained acceptance as a relevant animal model to evaluate efficacy of vaccines against B. anthracis. The plasmid constructs described in Examples 3-5, as well as similar plasmid constructs comprising native coding regions encoding native PA and LF, as well as empty control plasmids, are tested in vivo in rabbits by the following method. Plasmid vaccination of rabbits is done at four-week intervals. At each time point, the animals (n=2-4) receive an IM injection (quadriceps) of 500 μg (1 mg/ml) of DNA in 150 mM sodium phosphate formulated with the adjuvant Vaxfectin™ at a 4:1 DNA:Vaxfectin™ mass ratio, each animal receiving a total of 3 injections (1500 μg/animal). Alternative DNA formulations include other adjuvants as described herein, for example, CRL1005/BAK (see Shriver, J. W. et al., Nature 415:331-335 (2002), and P.C.T. Publication No. WO 02/00844 A2), and/or transfection-facilitating cationic lipids, e.g., DMRIE/DOPE at a 4:1 DNA:lipid mass ratio. Serum samples are taken at Day 0, 42, and 69 to determine antibody titers. The animals receive an aerosolized challenge on Day 70.

Rabbits are challenged in a BSL-3 facility (available, for example, at the Battelle Medical Research Evaluation Facility (MREF) in West Jefferson, Ohio) by standard methods. See, e.g., Henderson, D W J. Hygiene 50:53-68 (1952)). The Battelle facility has the equipment, staff, and certification to safely conduct a aerosol challenge of large mammals using infectious and toxin producing B. anthracis. Vaccinated animals are transferred to Battelle's facility in West Jefferson, and then, after a IACUC approved holding isolation period, the animals are challenged with between 50 and 100 LD50 aerosolized B. anthracis spores by inhalation. The animals are monitored for morbidity and mortality at regular intervals following challenge.

c) Non-Human Primate Immunizations.

The plasmid constructs described in Examples 3-5, as well as similar plasmid constructs comprising native coding regions encoding native PA and LF, as well as empty control plasmids, are tested in vivo in non-human primates by the following method. Cynomolgus macaques (M. fascicularis) are used for immunization and challenge experiments. Plasmid vaccination of the macaques is done at four-week intervals. Animals receive 1 to 1.5 mg each of DNA at each immunization bilaterally (2 to 3 mg total) intramuscularly, in the deltoid muscle. Following immunization, all animals are challenged by pulmonary delivery of aerosolized B. anthracis.

d) Human Immunizations.

The plasmid constructs described in Examples 3-5, as well as similar plasmid constructs comprising native coding regions encoding native PA and LF, as well as empty control plasmids, are tested in vivo in healthy human volunteers by the following method. The plasmids are formulated in 150 mM sodium phosphate, optionally including Vaxfectin™ at a 4:1 DNA:Vaxfectin™ mass ratio, and or a poloxamer, e.g., 0.01% (w/v) Pluronic® R 25R2. Vaccinations are given at 0, 4, and 8 weeks intramuscularly into the deltoid muscle either by needle injection or by needleless Biojector jet (see, e.g., Wang, R. et al. Proc. Natl. Acad. Sci. USA 98:10817-10822 (2001)). The volunteers receive 1 to 1.5 mg each of DNA at each immunization. Following immunization, serum specimens are collected from the volunteers and tested for antibodies to B. anthracis LF or PA.

E) Laboratory Animal, Companion Animal, or Food Animal Immunizations.

Plasmid constructs such as those described in Examples 3-5, are prepared using codon-optimized coding regions optimized for the species of interest using an appropriate codon-usage table, e.g., Table 3 (mouse), Table 4 (domestic cat), or Table 5 (cow). Codon optimization may be carried out by using relative frequencies for the codons, or by using the most frequent codon, as described herein. Plasmids comprising these coding regions, plus similar plasmid constructs comprising native coding regions encoding native PA and LF, as well as empty control plasmids, are tested in vivo in various animal species by the following method. The animal species of interest is immunized with an appropriate amount of a DNA vaccine codon-optimized for that species, at an appropriate amount, delivered in an appropriate route for that species, including, but not limited to the following immunization strategies: for mouse immunization, intramuscular delivery into the rectus femoris muscle of 50 μg DNA in 150 mM sodium phosphate (1 mg/ml)/leg at 0, 14 and 28 days; for cow immunization, intradermal delivery into the ear of 500 μg DNA in normal saline (1 mg/ml) at days 0 and 21 (see, e.g, van Drunen Little-van den Hurk et al. J. Gen. Virol. 79:831-839 (1998)); and for domestic cat immunization, intradermal delivery of 300 μg DNA in normal saline (1 mg/ml) at days 0, 15, and 30 (see, e.g., Osorio, J E, et al. Vaccine 17:1109-1116 (1999)).

### Example 9

### Immunological Assays

a) ELISA for LF and PA Antibody Titers.

Microtiter plates are coated with either PA or LF antigen by incubating 100 ng/well of purified protein (obtained from List Biological Laboratories, Campbell, Calif.) overnight at 4° C. in 100 mM carbonate buffer, pH 9.6. The wells are washed (3×) with 10 mM Tris-buffered (pH 7.3); 150 mM NaCl (TBS) followed by a 1% (w/v) BSA block. Serially diluted experimental and control serum samples in TBS+0.05% Tween are added to the wells and incubated for 60 min at room temperature. Enzyme conjugated (horseradish peroxidase or alkaline phosphatase) anti-mouse, anti-rabbit, or anti-monkey IgG are then added to each well and supernatants monitored for enzyme product. Antibody titers are defined as the highest dilution of a serum sample that results in an absorbance value 2× greater than that of a non-immune control serum. Antibody quantification will be determined using a purified anti-PA and anti-LF IgG1 and IgG2 reagent antibody.

b) Toxin Neutralization Assay.

Antibodies from vaccinated animals are initially tested using an in vitro assay that measures the neutralization of lethal toxin (Letx, i.e., LF and PA protein) cytotoxicity. Briefly, this protection assay is carried out using 24 hr. cultures of J774A.1 mouse macrophage cells maintained in microtiter plates (˜6×10⁴ cells per well) in DME media, with glucose and L glutamine supplements, and 7% fetal bovine serum at 37° C. Serially diluted serum from vaccinated and control animals are mixed with letx and allowed to sit for 60 min. The final Letx concentration will be brought to 3 μg/ml. This mixture will then added to the J774A.1 cells and incubated for seven hours at 37° C. Finally, 100 μl of 0.5 mg/ml 3-[4,5-dimethylthiazol-2yl]-2,5-diphenyltetrazolium bromide (MTT) is added to each experimental well and allowed to incubate another 60 min. before assaying for cytotoxicity. In this assay, surviving cells metabolize MTT into an insoluble purple pigment in a manner that is proportional to viability. This insoluble pigment is recovered from viable cells and quantitated by absorption of 450 nm light.

### Example 10

### Immunization Using a Prime-Boost Strategy

There is accumulating evidence to suggest that a naked DNA prime with a heterologous viral or protein boost will result in an enhanced humoral response. Since the humoral response is widely believed to be the immune correlate of protection against B. anthracis, in certain experiments a prime-boost strategy is used. The boost may be purified non-toxic LF and/or PA protein or the commercially available AVA vaccine. Alternatively recombinant virus vectors, e.g., adenovirus vectors, expressing non-toxic LF and/or PA may be used as the boost. Results are evaluated to compare antibody titers resulting from prime-boost immunization relative to DNA vaccination alone.

New Zealand rabbits are immunized with a series of three plasmid injections or two plasmid injections with the plasmid constructs described in Examples 3-5, as well as similar plasmid constructs comprising native coding regions encoding native PA and LF, as well as empty control plasmids, followed by a single dose of recombinant PA and/or LF protein (1 microgram in Alhydrogel) or the AVA vaccine (5 microliters). Controls include immunization with the codon optimized and control plasmid constructs alone, and mock immunizations. Following the immunization series, e.g., two plasmid DNA immunizations at four week intervals followed by a boost at week 12, total antibody titers and neutralizing titers are determined. In addition, selected immunized animals are challenged with a 500×LD50 dose of aerosolized anthrax spores at Battelle Medical Research Evaluation-Facility in West Jefferson, Ohio as described in Example 8.

### Example 11

### Immunization of Mice Using Codon-Optimized B. anthracis DNA Vaccines

a) Experiment 1

Six groups (Groups A-F) of 5 Balb/c female mice were injected bilaterally in the rectus femoris muscle with 50 μl of DNA solution (at 1.0 mg/ml) (100 μl total/mouse), on days 1 and 21 and 42 with each of the following plasmids:
Group 1A: VR6290 (TPA-PA63, FIG. 1, SEQ ID NO:1, prepared as described in Example 3a);
Group 1B: VR6291 (TPA-PA63ΔFF, FIG. 2, SEQ ID NO:5, prepared as described in Example 3b);
Group 1C: VR6292 (TPA-PA83Δfurin, FIG. 3, SEQ ID NO:7, prepared as described in Example 3c);
Group 1D: VR6295 (TPA-LF HEXXH, FIG. 4, SEQ ID NO:9, prepared as described in Example 4a);
Group 1E: VR6290 (50 μg)+VR6295 (50 μg), co-injected; and
Group 1F: VR1012 (empty expression vector).

The plasmids listed above were formulated as follows. One vial (0.5 mg) of MPL+TDM adjuvant, purchased from Sigma/Aldrich (catalog #M6536) was resuspended in 150 mM Na₂PO₄ according to manufacturers instructions. Fifty microliters of DNA solution was mixed 1:1 (v/v) with the MPL+TDM emulsion and injected into each mouse at the times specified above.

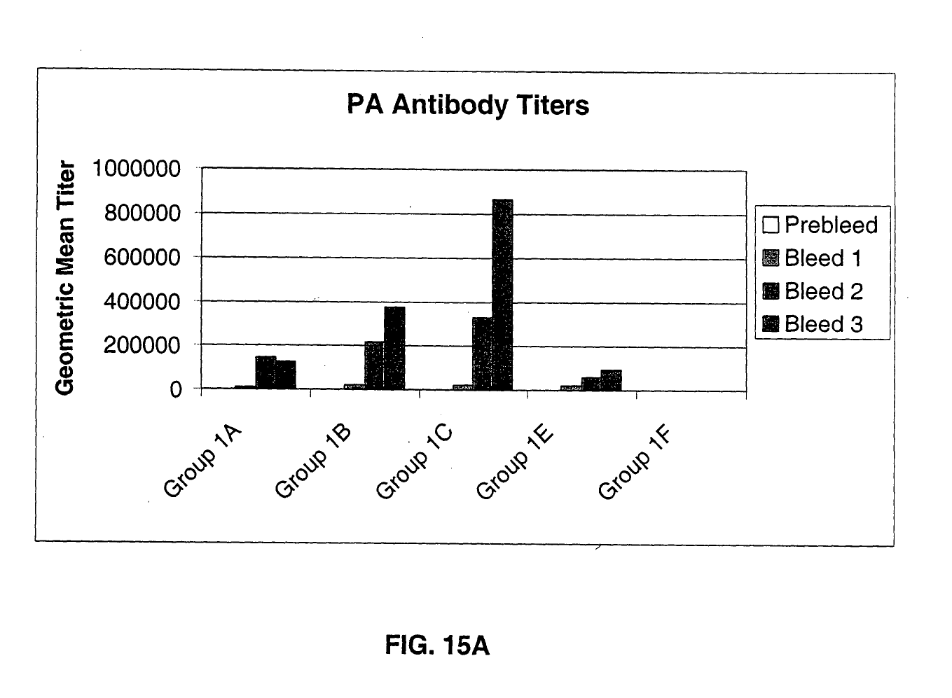

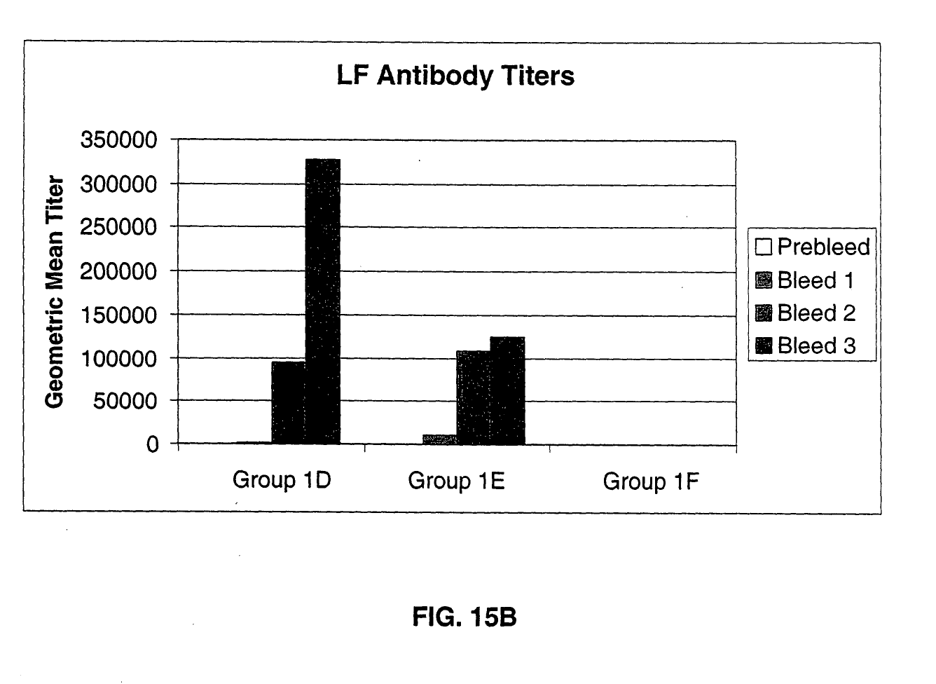

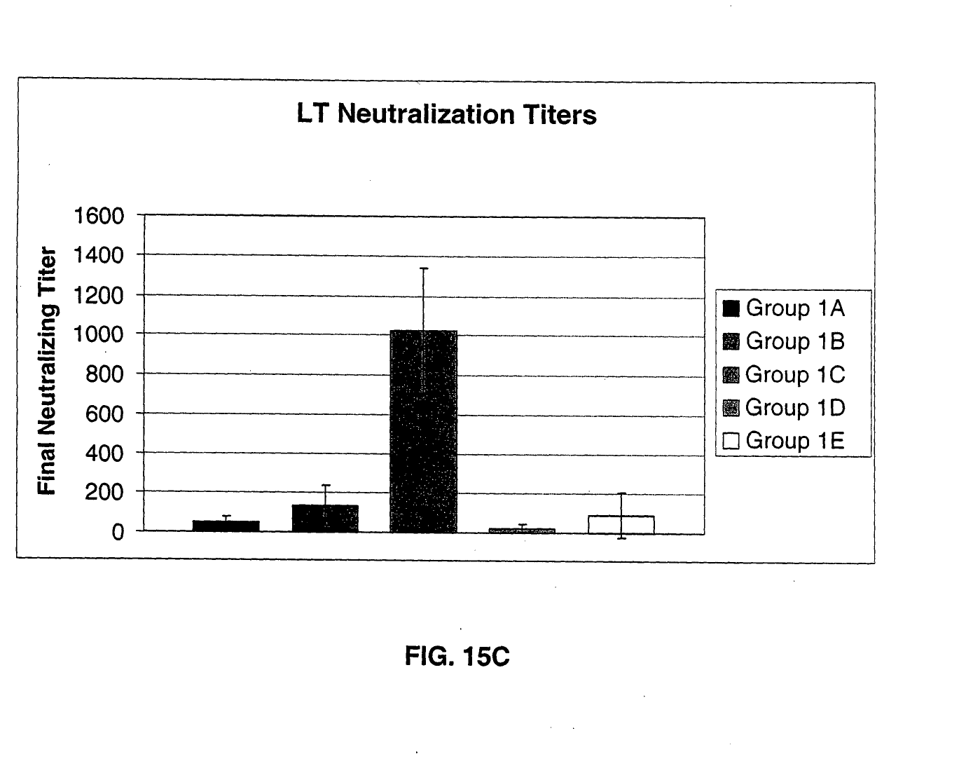

Mice were bled for serum on days 0 (prebleed), 20 (bleed 1), and 41 (bleed 2), and 62 (bleed 3). PA antibodies were measured in each of Groups 1A-1C, 1E, and 1F, LF antibodies were measured in each of Groups 1D, 1E, and 1F, and LT neutralizing antibodies were measured in each of Groups 1A-1E. All assays were done as outlined in Example 9. The geometric mean of the anti-PA and anti-LF titers were calculated following each bleed. The results are shown in FIGS. 15A and 15B, respectively. In FIG. 15C, the serum from each mouse was tested for LT neutralizing antibody titer after the last DNA immunization (bleed 3) according to the procedure in Example 9. The mean neutralizing titer for each group of mice was calculated and plotted and the error bars represent one standard deviation from the mean.

b) Experiment 2

Eight groups of 5 mice each (Groups 2A-2H) were injected bilaterally in the rectus femoris with 50 μl (50 μg) of DNA solution (100 μl (100 μg) total per mouse), adjuvanted with MPL+TDM as described in section 11a, on days 1, 21, and 49 with the following combinations of plasmids:
Group 2A: VR-6290 (50 μg)+VR-1012 (50 μg);
Group 2B: VR-6291 (50 μg)+VR-1012 (50 μg);
Group 2C: VR-6292 (50 μg)+VR-1012 (50 μg);
Group 2D: VR-6295 (50 μg)+VR-1012 (50 μg);
Group 2E: VR-6290 (50 μg)+50 μg VR-6295
Group 2F: VR-6291 (50 μg)+VR-6295 (50 μg);
Group 2G: VR-6292 (50 μg)+VR-6295 (50 μg); and
Group 2H: VR-1012 (100 μg).

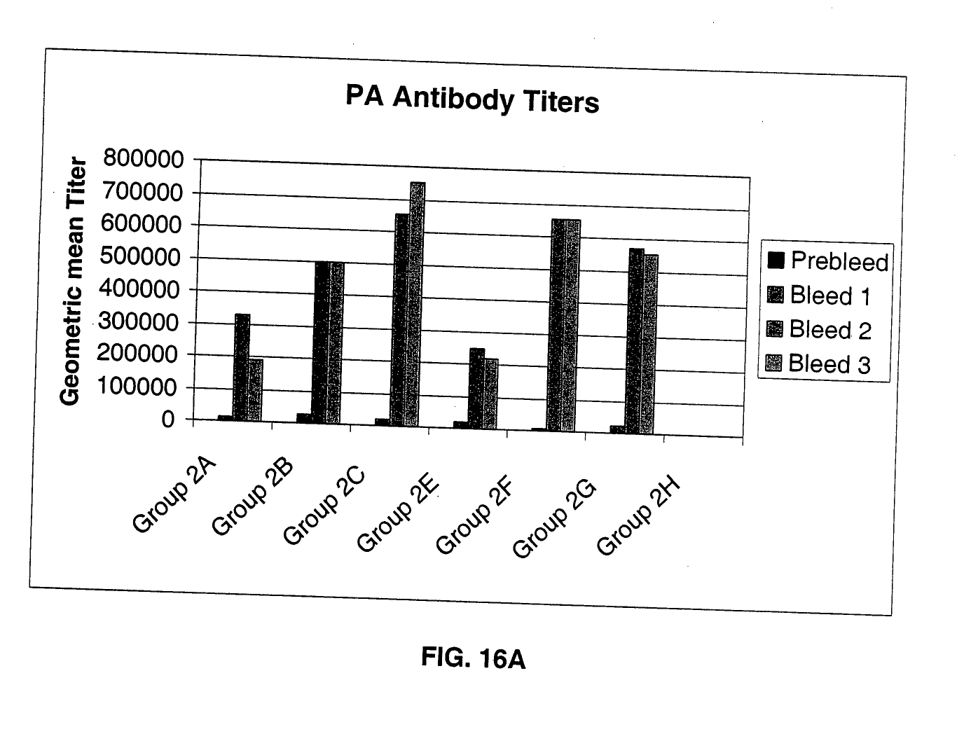

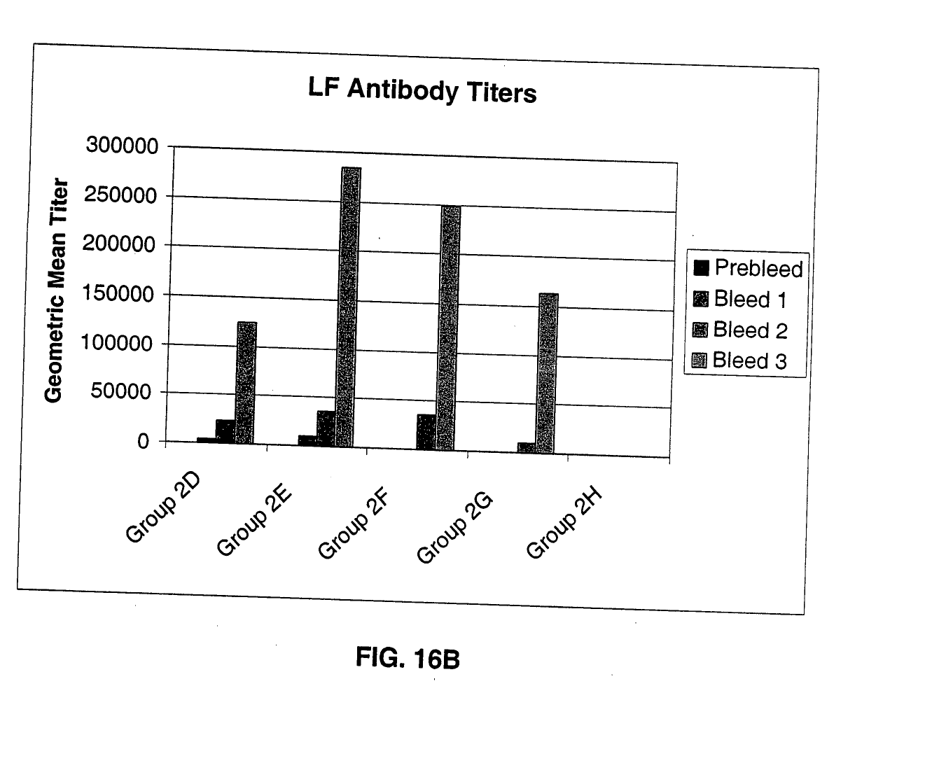

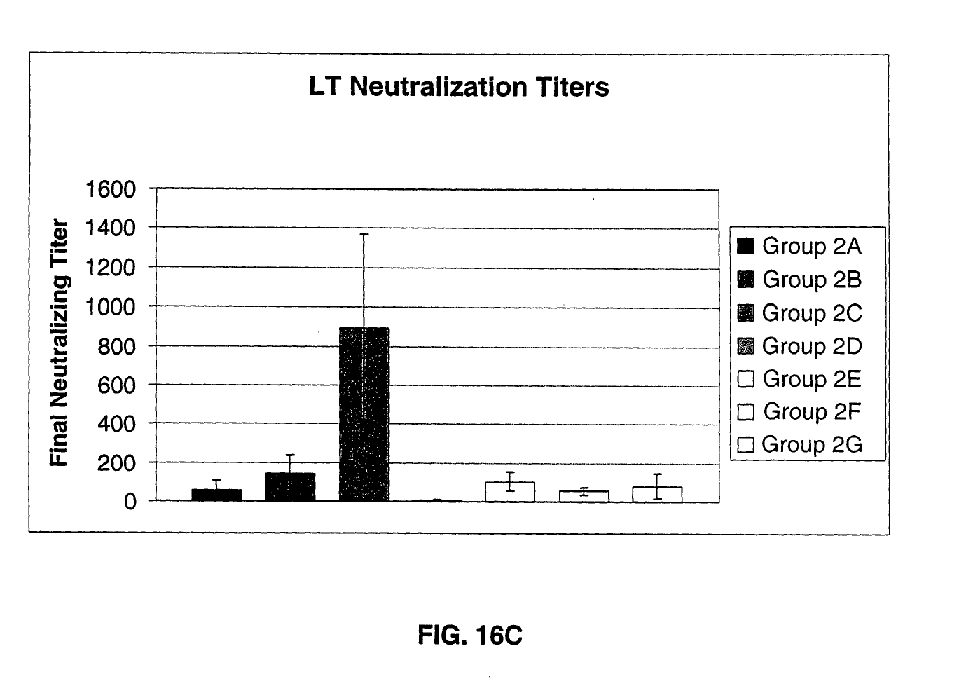

Mice were bled for serum on days 0 (prebleed), 20 (bleed 1), and 41 (bleed 2), and 62 (bleed 3). PA antibodies were measured in each of Groups 2A-2C and 2E-2H, LF antibodies were measured in each of Groups 2D-2H, and LT neutralizing antibodies were measured in each of Groups 2A-2G. All assays were done as outlined in Example 9. The geometric mean of the anti-PA and anti-LF titers were calculated following each bleed. The results are shown in FIGS. 16A and 16B, respectively. In FIG. 16C, the serum from each mouse was tested for LT neutralizing antibody titer after the last DNA immunization (bleed 3) according to the procedure in Example 9. The mean neutralizing titer for each group of mice was calculated and plotted and the error bars represent one standard deviation from the mean.

c) Experiment 3

Four groups of 5 mice each (Groups 3A-3D) were injected bilaterally in the rectus femoris with 50 μl (50 μg) of DNA solution (100 μl (100 μg) total per mouse), adjuvanted with MPL+TDM as described in section 11a, on days 1, 21, and 49 with the following combinations of plasmids:
Group 3A: VR-6292 (50 μg)+VR-1012 (50 μg);
Group 3B: VR-6292 (50 μg)+VR-62952 (50 μg, TPA-LF Domain I-III, FIG. 5, SEQ ID NO:13, prepared as described in Example 4b);
Group 3C: VR-6292 (50 μg)+VR-62951 (50 μg, TPA-LF Domain IB, FIG. 14, SEQ ID NO:39, prepared as described in Example 4d); and
Group 3D: VR-6299 (50 μg, TPA-Sugar minus PA63, FIG. 7, SEQ ID NO:17, prepared as described in Example 5a)+VR-1012 (50 μg).

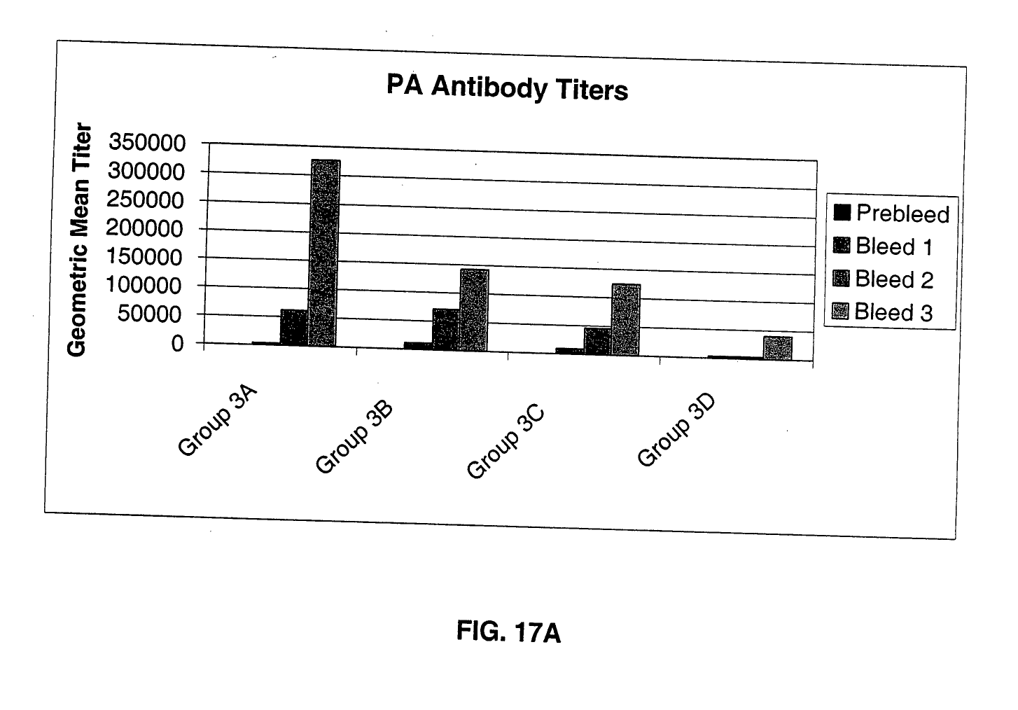

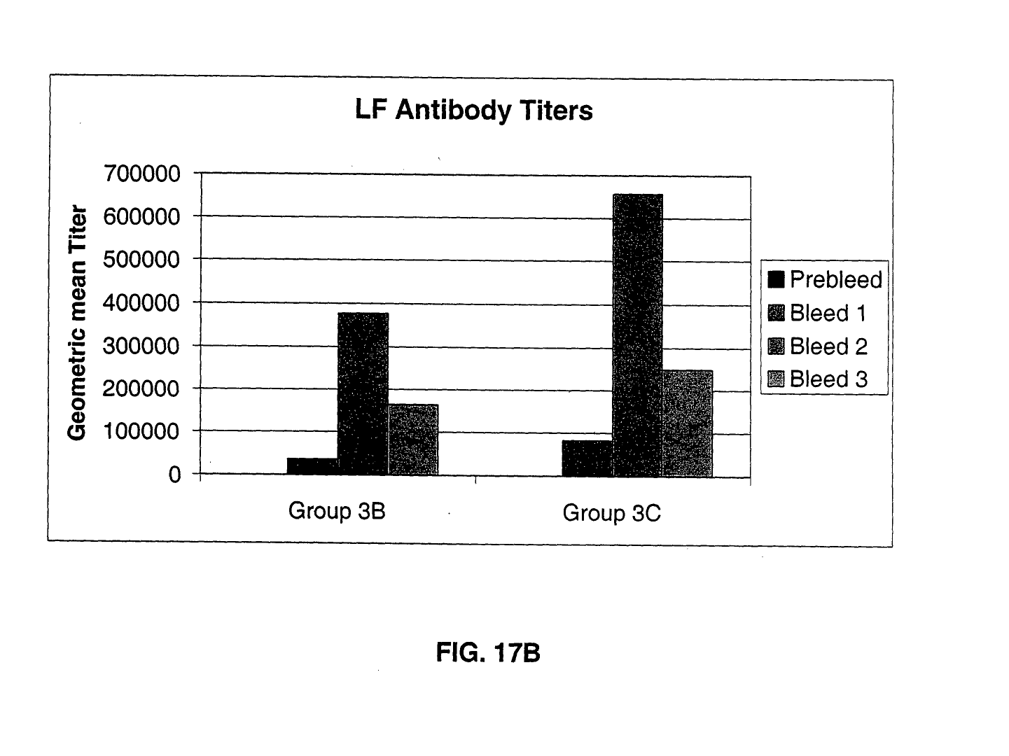

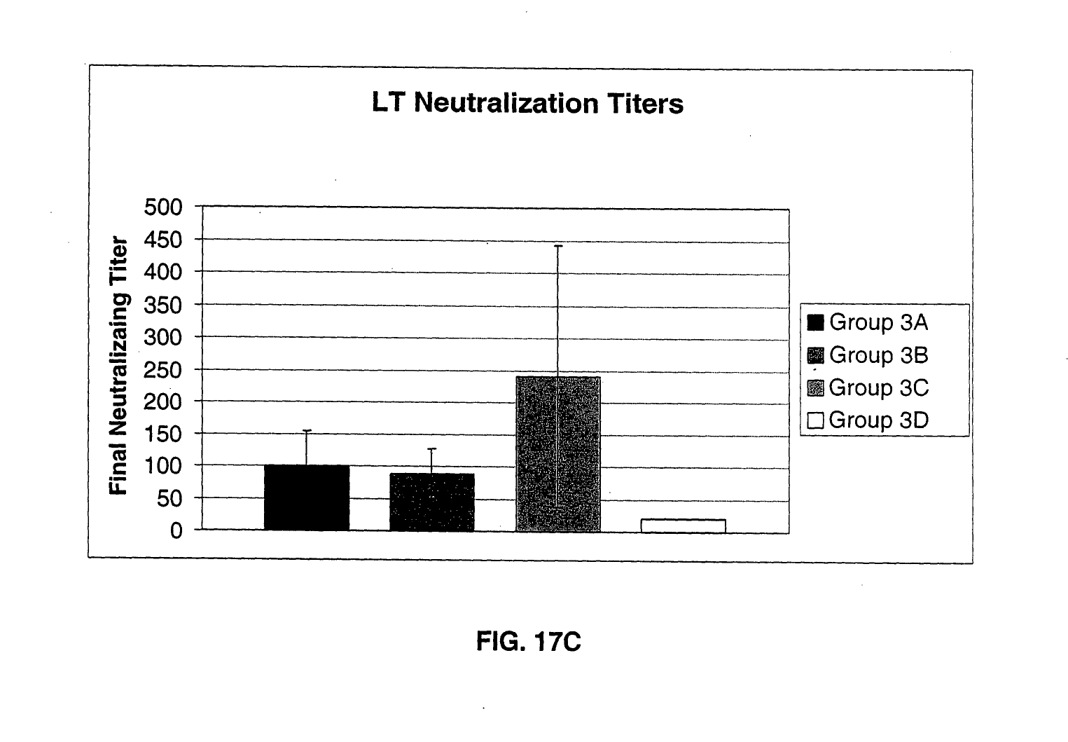

Mice were bled for serum on days 0 (prebleed), 20 (bleed 1), and 41 (bleed 2), and 62 (bleed 3). PA antibodies were measured in each of Groups 3A-3D, LF antibodies were measured in each of Groups 3B and 3C, and LT neutralizing antibodies were measured in each of Groups 3A-3D. All assays were done as outlined in Example 9. The geometric mean of the anti-PA and anti-LF titers were calculated following each bleed. The results are shown in FIGS. 17A and 17B, respectively. In FIG. 17C, the serum from each mouse was tested for LT neutralizing antibody titer after the last DNA immunization (bleed 3) according to the procedure in Example 9. The mean neutralizing titer for each group of mice was calculated and plotted and the error bars represent one standard deviation from the mean.

d) Experiment 4

Four groups of 10 mice each (Groups 4A-4D) were injected bilaterally in the rectus femoris with 50 μl (50 μg) of plasmid VR-6292 (100 μl (100 μg) total per mouse), formulated with various adjuvants, on days 1, 21, and 49, as follows:
Group 4A: VR-6292 formulated with CRL 1005/BAK;
Group 4B: VR-6292 formulated with MPL+TDM, as described in section 11a, supra;
Group 4C: VR-6292 formulated with Vaxfectin™ at a 4:1 DNA:Vaxfectin™ mass ratio; and
Group 4D: VR-6292 formulated with DMRIE:DOPE (1:1 molar ratio) at a 4:1 DNA:lipid mass ratio.

The plasmids in Group 4A were formulated as follows. The poloxamer CRL1005 (from Organichem) and a solution of benzyl-alkonium chloride “BAK” (from Ruger Chemicals) were added sequentially to plasmid solutions in PBS. The initial plasmid/poloxamer formulation was prepared to contain 5 mg/mL plasmid DNA, 7.5 mg/mL CRL1005 and 0.3 mM BAK. These initial preparations were diluted 1:1 (vol:vol) with PBS, then cold sterile filtered. Further dilution with sterile PBS was done just prior to use to provide the final working concentration of 1 mg/mL pDNA, 1.5 mg/mL CRL1005 and 0.06 mM BAK.

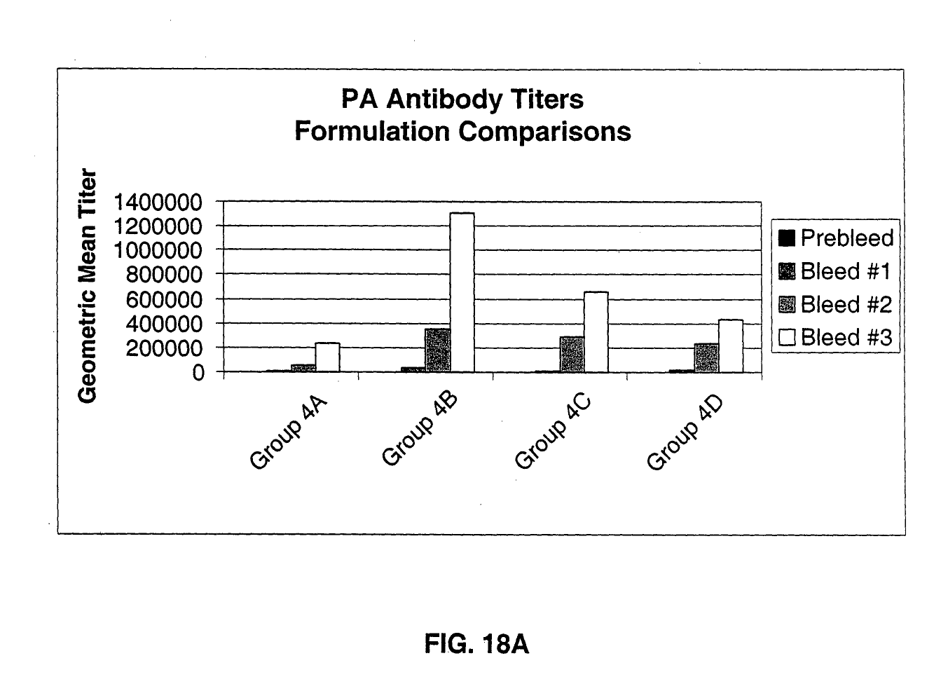

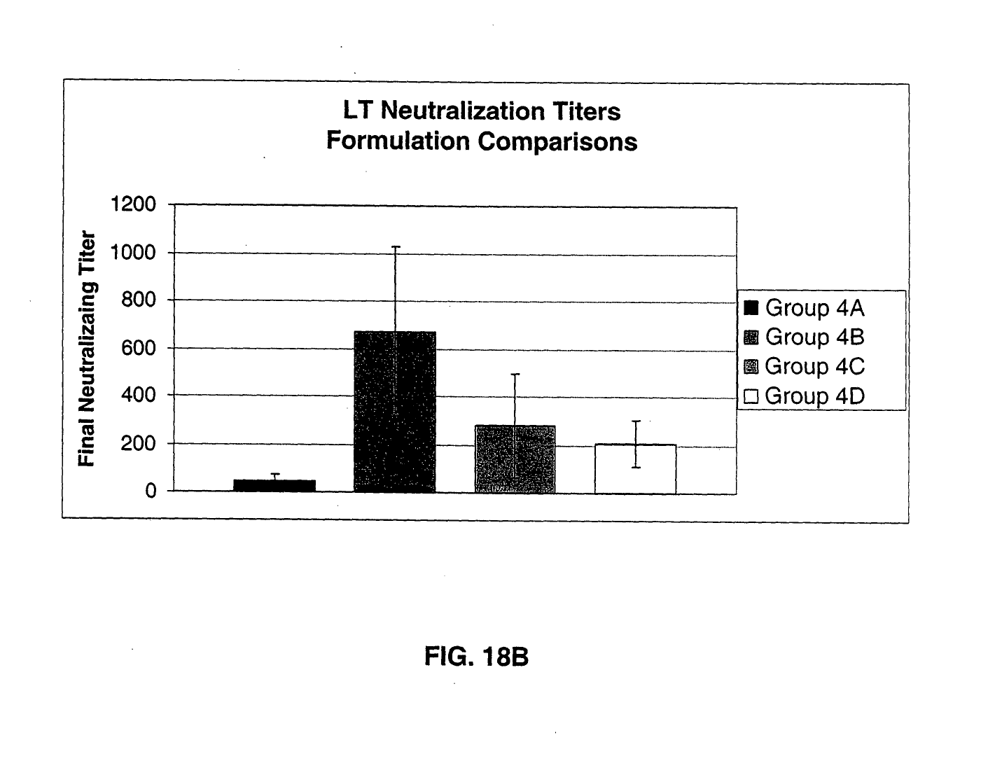

Mice were bled for serum on days 0 (prebleed), 20 (bleed 1), and 41 (bleed 2), and 62 (bleed 3). PA antibodies were measured in each of Groups 4A-4D, and LT neutralizing antibodies were measured in each of Groups 4A-4D. All assays were done as outlined in Example 9. The geometric mean of the anti-PA titers were calculated following each bleed. The results are shown in FIG. 18A. In FIG. 18B, the serum from each mouse was tested for LT neutralizing antibody titer after the last DNA immunization (bleed 3) according to the procedure in Example 9. The mean neutralizing titer for each group of mice was calculated and plotted and the error bars represent one standard deviation from the mean.

e) Experiment 5

Six groups of 10 mice each (Groups 5A-5F) were injected bilaterally in the rectus femoris with 50 μl (50 μg) of plasmid VR-6292 (100 μl (100 μg) total per mouse), formulated with various adjuvants, on days 1, 14, and 28, as follows:
Group 5A: VR-6292 formulated with MPL+TDM, as described in section 11a, supra;
Group 5B: VR-6292 formulated with MPL-A aqueous 1000 μg/mL (Corixa) mixed 1:1 (v/v) with DNA;
Group 5C: VR-6292 formulated with CRL 1005/BAK, as described in section 11d, supra;
Group 5D: VR-6292 formulated with Vaxfectin™ at a 4:1 DNA:Vaxfectin™ mass ratio;
Group 5E: VR-6292 formulated with DMRIE:DOPE (1:1 molar ratio) at a 4:1 DNA:lipid mass ratio; and
Group 5F: VR-6292 formulated with 1×PBS.

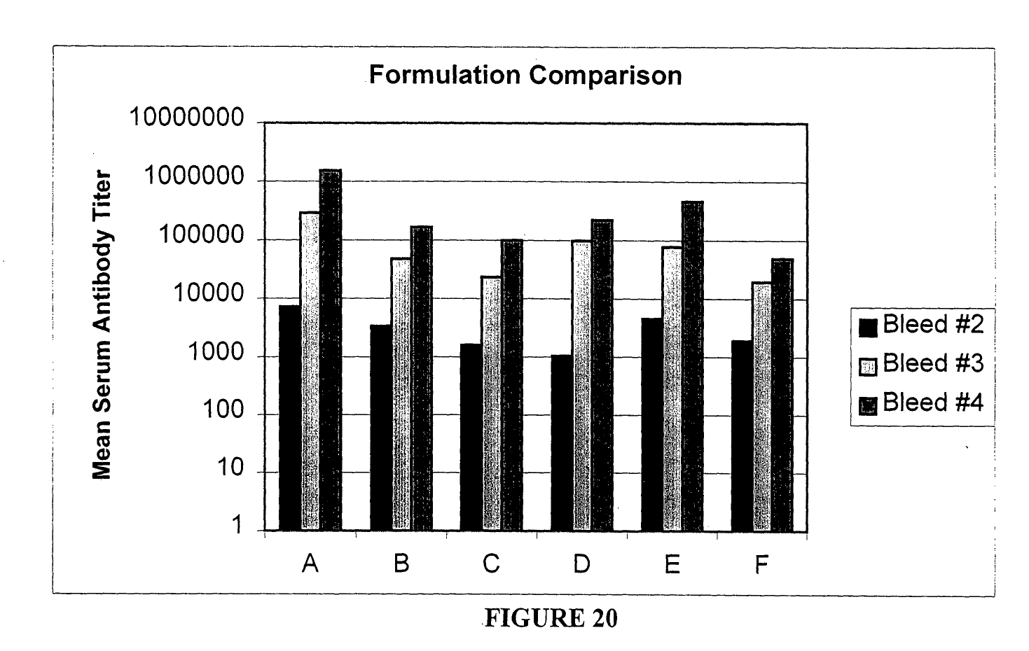

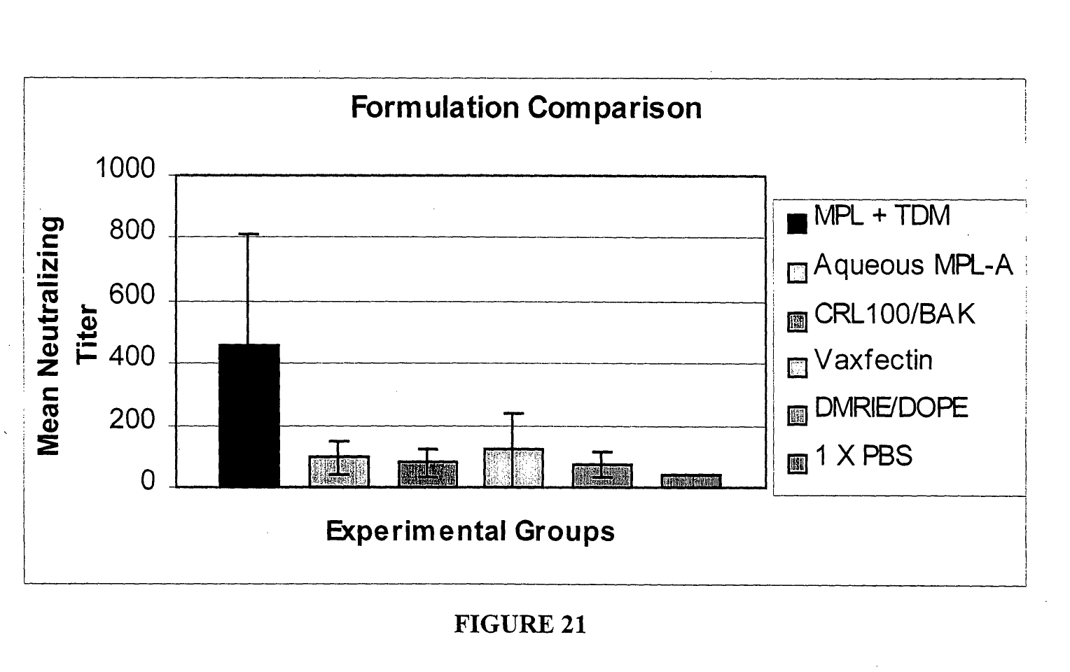

Mice were bled for serum on days 0 (prebleed), 13 (bleed 1), and 27 (bleed 2), and 56 (bleed 3). PA antibodies were measured in each of Groups 5A-5F after each bleed, and LT neutralizing antibodies were measured in each of Groups 5A-5F after bleed 3. All assays were done as outlined in Example 9. The geometric mean of the anti-PA titers were calculated following each bleed. The results are shown in FIG. 20. In FIG. 21, the serum from each mouse was tested for LT neutralizing antibody titer after the last DNA immunization (bleed 3) according to the procedure in Example 9. The mean neutralizing titer for each group of mice was calculated and plotted and the error bars represent one standard deviation from the mean.

### Example 12

### Immunization of Rabbits Using Codon-Optimized B. anthracis DNA Vaccines

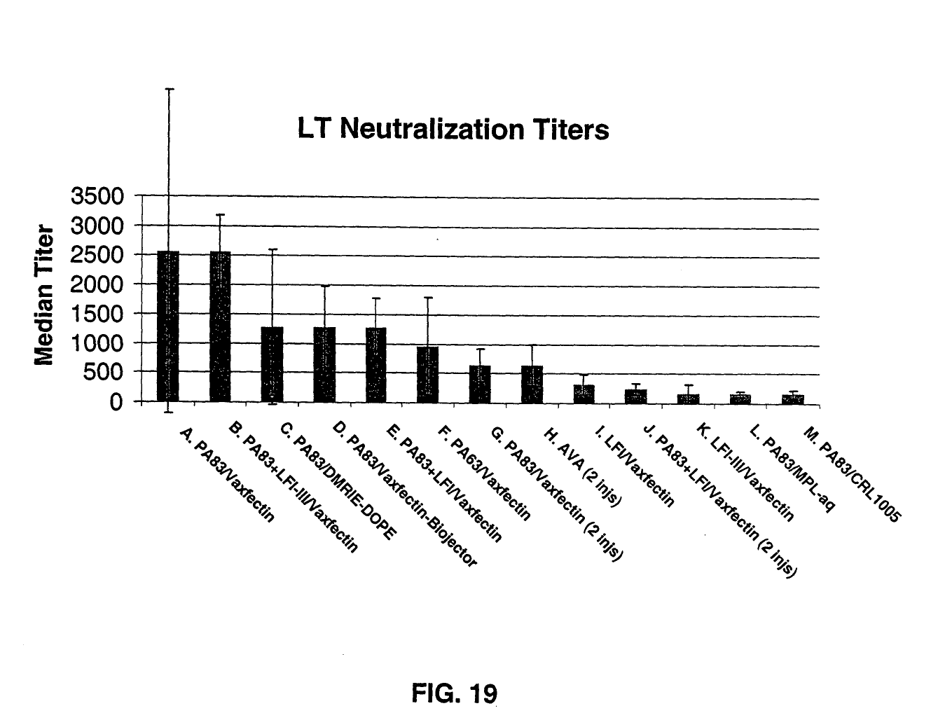

Twelve (12) groups of 10 rabbits each (Groups A-G and I-M, for DNA vaccinations) and one group of 4 rabbits (Group H, for the AVA vaccination) (Oryctolagus cuniculus, New Zealand albino rabbits, 2-5 kg each at onset of treatment) were used in this experiment. The rabbits in Groups A-G and I-M received a 500 μg intramuscular injection in each quadricep muscle (bilateral) for a total of 1 mg of plasmid DNA per rabbit per immunization. Injection of the formulated plasmid DNA took place on days 0, 28, and 56. Some animals received only the first two plasmids injections on days 0 and 28 (denoted 2 injs in FIG. 19). All rabbits were prebled two days before the first immunization and bled again on days 14, 42, and 70.

Unless noted, the various formulations were administered by a bilateral intramuscular injection into the quadriceps muscles on Days 0, 28, and 56 with a needle. The dose volume to be administered is 500 μl/muscle, 1 ml/animal. The rabbits in Group D were vaccinated using a Biojector, as follows. Animals were anesthetized using ketamine/xylazine. The skin over the injection site was shaved, and the dose volume administered was 500 μl/muscle, 1 ml/animal. The vaccination groups were as follows:
Group A: VR6292 formulated with Vaxfectin™ at a 4:1 DNA:Vaxfectin™ ratio;
Group B: VR6292 (500 μg)+VR-62952 (500 μg) formulated with Vaxfectin™ at a 4:1 DNA:Vaxfectin™ ratio;
Group C: VR6292 formulated with DMRIE/DOPE at a 4:1 DNA:lipid ratio;
Group D: VR6292 formulated with Vaxfectin™ at a 4:1 DNA:Vaxfectin™ ratio, delivered by Biojector;
Group E: VR6292 (500 μg)+VR-62951 (500 μg) formulated with Vaxfectin™ at a 4:1 DNA:Vaxfectin™ ratio;
Group F: VR6290 formulated with Vaxfectin™ at a 4:1 DNA:Vaxfectin™ ratio;
Group G: VR6292 formulated with Vaxfectin™ at a 4:1 DNA:Vaxfectin™ ratio (two injections only);
Group H: Commercial anthrax vaccine AVA, 50 μl, delivered on day 28 and 56 by a single IM injection;
Group I: VR-62951 formulated with Vaxfectin™ at a 4:1 DNA:Vaxfectin™ ratio;
Group J: VR6292 (500 μg)+VR-62951 (500 μg) formulated with Vaxfectin™ at a 4:1 DNA:Vaxfectin™ ratio (two injections only);
Group K: VR-62952 formulated with Vaxfectin™ at a 4:1 DNA:Vaxfectin™ ratio;
Group L: VR6292 formulated with MPL-A aqueous 1000 μg/mL (Corixa) mixed 1:1 (v/v) with DNA;
Group M: VR6292 formulated with CRL1005/BAK, formulated as described in Example 11d, supra.

The LT neutralization assay was performed on all rabbit sera from the day 70 bleed. The median titer±one standard deviation is shown for each group in FIG. 19.

### Example 13

### Immunization and Challenge of Rabbits Using Codon-Optimized B. anthracis DNA Vaccines

Ten groups of rabbits (Oryctolagus cuniculus, New Zealand albino rabbits, 2-5 kg each at onset of treatment, ten (10) animals per group unless otherwise noted) were used in this experiment. These included selected groups of animals described in Example 12, as noted below. The various plasmid DNA formulations were administered by a bilateral intramuscular injection into the quadriceps muscles on Days 0, 28, and 56 with a needle. The dose volume to be administered is 500 μl/muscle, 1 ml/animal. The vaccination groups were as follows:
Group 1: VR6292 formulated with Vaxfectin™ at a 4:1 DNA:Vaxfectin™ ratio (Group A from Example 12);
Group 2: VR6292 formulated with Vaxfectin™ at a 4:1 DNA:Vaxfectin™ ratio (two injections only) (Group G from Example 12);
Group 3: VR6292 formulated with DMRIE/DOPE at a 4:1 DNA:lipid ratio (Group C from Example 12);
Group 4: VR6292 (500 μg)+VR-62951 (500 μg) formulated with Vaxfectin™ at a 4:1 DNA:Vaxfectin™ ratio (two injections only) (Group J from Example 12);
Group 5: VR6292 (500 μg)+VR-62952 (500 μg) formulated with Vaxfectin™ at a 4:1 DNA:Vaxfectin™ ratio (Group B from Example 12);
Group 6: VR-62952 formulated with Vaxfectin™ at a 4:1 DNA:Vaxfectin™ ratio (two animals) (Group K from Example 12);
Group 7: VR1012 formulated with Vaxfectin™ at a 4:1 DNA:Vaxfectin™ ratio (four animals);
Group 8: VR1012 formulated with DMRIE/DOPE at a 4:1 DNA:lipid ratio (two animals);
Group 9: Commercial anthrax vaccine AVA, 50 μl, delivered on 28 and 56 by a single IM injection (Group I from Example 12); and
Group 10: Twelve unvaccinated animals.

The rabbits in Groups 1-8 received a 500 μg intramuscular injection in each quadricep muscle (bilateral) for a total of 1 mg of plasmid DNA per rabbit per immunization. In groups 1, 3, 5, 6, 7, 8, and 9, three injections of the formulated plasmid DNA took place on days 0, 28, and 56. In groups 2 and 4, two injections of the formulated plasmid DNA took place on days 0 and 28. In group 19 commercial anthrax vaccine AVA, 50 μl, was injected intramuscularly on days 28 and 56. All rabbits were prebled two days before the first immunization and bled again on days 14, 42, and 70.

Over a four-day period on or around day 70 (indicated in Table 17 as “challenge days” C1-C4), the rabbits were challenged by aerosol administration of B. anthracis (Ames strain) spores by standard methods. See, e.g., Henderson, D W J. Hygiene 50:53-68 (1952)). Challenge doses ranged from about 50 LD50 equivalents to about 250 LD50 equivalents as noted in Table 17 below. The animals were monitored for morbidity and mortality at regular intervals out to days 19-22 (depending on the challenge day) following challenge. The results are shown in Table 17, and are summarized in Table 18. “NC” denotes “not challenged.”

TABLE 17

| | | | |
|---|---|---|---|
| | CHALLENGE | AMES LD50 | |
| ANIMAL ID | DAY | EQUIVALENT | SURVIVAL |

Group 1

| | | | |
|---|---|---|---|
| 1.1 | C3 | 123.5 | Y |
| 1.2 | NC | | |
| 1.3 | C4 | 113.5 | Y |
| 1.4 | C1 | 56.4 | Y |
| 1.5 | C3 | 92.7 | Y |
| 1.6 | C4 | 66.3 | Y |
| 1.7 | C1 | 103 | Y |
| 1.8 | C2 | 127.3 | Y |
| 1.9 | NC | | |
| 1.10 | C2 | 128.8 | Y |

Group 2

| | | | |
|---|---|---|---|
| 2.1 | C1 | 76 | Y |
| 2.2 | C4 | 70.5 | Y |
| 2.3 | NC | | |
| 2.4 | C4 | 52.1 | Y |
| 2.5 | C1 | 252.1 | Y |
| 2.6 | C2 | 119.1 | Y |
| 2.7 | C3 | 52.4 | Y |
| 2.8 | NC | | |
| 2.9 | C2 | 71.9 | Y |
| 2.10 | C3 | 195.1 | Y |

Group 3

| | | | |
|---|---|---|---|
| 3.1 | C2 | 55.7 | Y |
| 3.2 | C3 | 238.3 | Y |
| 3.3 | C4 | 110 | Y |
| 3.4 | C1 | 208.1 | Y |
| 3.5 | NC | | |
| 3.6 | C1 | 142.9 | Y |
| 3.7 | C3 | 169 | Y |
| 3.8 | NC | | |
| 3.9 | C4 | 57.5 | Y |
| 3.10 | C2 | 74.7 | Y |

Group 4

| | | | |
|---|---|---|---|
| 4.1 | C3 | 87.3 | Y |
| 4.2 | C4 | 90.2 | Y |
| 4.3 | C1 | 81.6 | Y |
| 4.4 | NC | | |
| 4.5 | C2 | 100 | Y |
| 4.6 | C2 | 72 | Y |
| 4.7 | C1 | 76.1 | Y |
| 4.8 | C4 | 92.8 | Y |
| 4.9 | NC | | |
| 4.10 | C3 | 205 | Y |

Group 5

| | | | |
|---|---|---|---|
| 5.1 | NC | | |
| 5.2 | C1 | 192.2 | Y |
| 5.3 | C2 | 152.6 | Y |
| 5.4 | C4 | 66.6 | Y |
| 5.5 | C3 | 135.7 | Y |
| 5.6 | C2 | 65.1 | Y |
| 5.7 | C4 | 79 | Y |
| 5.8 | C1 | 126.6 | Y |
| 5.9 | C3 | 154.7 | Y |
| 5.10 | NC | | |

Group 6

| | | | |
|---|---|---|---|
| 6.1 | C4 | 117.7 | Y |
| 6.2 | C3 | 241.4 | N (D4) |
| 6.3 | NC | | |
| 6.4 | C1 | 107.3 | Y |
| 6.5 | C4 | 58.7 | N (D4) |
| 6.6 | C3 | 121 | Y |
| 6.7 | C3 | 160.8 | Y |
| 6.8 | C2 | 46.1 | N (D7) |
| 6.9 | C1 | 195.2 | N (D6) |
| 6.10 | C2 | 94.5 | Y |

Group 7

| | | | |
|---|---|---|---|
| 7.1 | C3 | 101.9 | N (D3) |
| 7.2 | C4 | 144.1 | N (D2) |
| 7.3 | NC | | |
| 7.4 | C1 | 108.2 | N (D2) |

Group 8

| | | | |
|---|---|---|---|
| 8.1 | C2 | 63 | N (D3) |
| 8.2 | C4 | 58.2 | N (D3) |

Group 9

| | | | |
|---|---|---|---|
| 9.1 | C2 | 113.4 | Y |
| 9.2 | C1 | 106.9 | Y |
| 9.3 | C3 | 157.6 | Y |
| 9.4 | C4 | 175.6 | Y |

Group 10

| | | | |
|---|---|---|---|
| 10.1 | C4 | 76.7 | N (D2) |
| 10.2 | C3 | 207.6 | N (D3) |
| 10.3 | C2 | 91.5 | N (D2) |
| 10.4 | C4 | 176 | N (D2) |
| 10.5 | C2 | 123 | N (D3) |
| 10.6 | C2 | 95.4 | N (D3) |
| 10.7 | C4 | 91.5 | N (D3) |
| 10.8 | C1 | 165.2 | N (D2) |
| 10.9 | C1 | 57.3 | N (D3) |
| 10.10 | C3 | 163.8 | N (D4) |
| 10.11 | C3 | 114.2 | N (D2) |
| 10.12 | C1 | 62.3 | N (D2) |

TABLE 18

| Group | Survival |
|---|---|
| 1 | 8/8 (100%) |
| 2 | 8/8 (100%) |
| 3 | 8/8 (100%) |
| 4 | 8/8 (100%) |
| 5 | 8/8 (100%) |
| 6 | 5/9 (56%) |
| 7 | 0/2 (0%) |
| 8 | 0/3 |
| 9 | 4/4 (100%) |
| 10 | 0/12 (0%) |

### Example 14

### Immunization of Mice Using Single Vial Formulations

Single vial formulations were prepared by reconstituting bulk DMRIE and DOPE liquids to form multi-lamellar vesicles (MLV). These vesicles were then further processed to produce small DMRIE and DOPE liposomes (SUV) and sterile filtered through a 0.2 μm membrane. The formulations were prepared aseptically at room temperature by adding sterile plasmid DNA and sterile DMRIE:DOPE SUV liposomes into separate feed lines and then combining into a third vessel via in-line mixing. Moderate rates of addition and moderate in-vessel mixing were used to form a lipid/plasmid DNA complex. Preparation of lipids and lipid/plasmid DNA complexes is described in Zelphati et al. Gene Therapy 5: 1277-1282 (1998) which is incorporated herein by reference in its entirety. The formulations described below contain final molar ratios 4:1 or 2:1 plasmid DNA to DMRIE.

Eight groups of mice, containing 10 mice in each group, were injected bilaterally in the rectus femoris muscle with the various formulations described below. Each injection contained 50 μg of purified plasmid VR6292 (PA83Δfurin) in a volume of 0.1 ml. At 0, 2 and 4 weeks the groups were injected with the following formulations, all containing 50 μg of VR6292 plasmid DNA (prepared as described in the plasmid DNA purification section prior to Example 1).
Group A: Unextruded MLV, in a 4:1 molar ratio of plasmid DNA to DMRIE, in PBS (pH 7.2). The formulation was freshly prepared just prior to injection.
Group B: Unextruded MLV, in a 4:1 molar ratio of plasmid DNA to DMRIE, in 10% sucrose and 10 mM sodium phosphate, pH 7.2. The formulation was freshly prepared just prior to injection.
Group C: 0.2 μm filter extruded (SUV) liposomes, in a 4:1 molar ratio of plasmid DNA to DMRIE, in 10% sucrose and 10 mM sodium phosphate (pH 7.2). The formulation was stored overnight at 2-8° C. prior to inoculation.
Group D: SUV liposomes, in a 4:1 ratio plasmid DNA to DMRIE, in 10% sucrose and 10 mM sodium phosphate, pH 7.2. The formulation was frozen prior to inoculation.
Group E: SUV liposomes, in a 4:1 molar ratio of plasmid DNA to DMRIE, in 10% sucrose and 10 mM sodium phosphate, pH 7.2. The formulation was lyophilized prior to inoculation.
Group F: Unextruded MLV, containing cholesterol in place of DOPE, in a 4:1 molar ratio of plasmid DNA to DMRIE, in 10% sucrose and 10 mM sodium phosphate, pH 7.2. The formulation was freshly prepared just prior to injection.
Group G: Unextruded MLV, in a 2:1 molar ratio of plasmid DNA to DMRIE, in PBS, pH 7.2. The formulation was freshly prepared just prior to injection.
Group H: SUV liposomes, in a 2:1 molar ratio of plasmid DNA to DMRIE, in 10% sucrose and 10 mM sodium phosphate, pH 7.2. The formulation was stored overnight at 2-8° C. prior to injection.

Mice were bled for serum prior to each DNA immunization at week 0 (Prebleed), week 2 (Bleed 1), week 4 (Bleed 2) and four weeks post the last injection (Bleed 3). Anti-PA IgG antibody titers and neutralization of lethal toxin (Letx) titers were performed as described in Example 9. The antibody titers and neutralization results for each bleed and every formulation tested are shown in Tables 19 and 20.

TABLE 19
Anti - PA IgG Titer

| | | Group | | | |
|---|---|---|---|---|---|
| | | A | B | C | D |
| Geometric | Prebleed | 80 | 80 | 80 | 80 |
| Mean | | | | | |
| | Bleed 1 | 10975 | 4165 | 7760 | 4457 |
| | Bleed 2 | 305736 | 66540 | 108094 | 62084 |
| | Bleed 3 | 1616014 | 376405 | 376405 | 655627 |
| Std. Dev. | Prebleed | 0 | 0 | 0 | 0 |
| | Bleed 1 | 11372 | 7630 | 23983 | 12421 |
| | Bleed 2 | 215705 | 58765 | 84998 | 51642 |
| | Bleed 3 | 639310 | 370406 | 343674 | 1200361 |
| | | Group | | | |
| | | E | F | G | H |
| Geometric | Prebleed | 80 | 80 | 80 | 80 |
| Mean | | | | | |
| | Bleed 1 | 7760 | 5487 | 9554 | 4457 |
| | Bleed 2 | 81920 | 71316 | 327680 | 76434 |
| | Bleed 3 | 1310720 | 266159 | 1310987 | 351199 |
| Std. Dev. | Prebleed | 0 | 0 | 0 | 0 |
| | Bleed 1 | 15017 | 8172 | 23498 | 26219 |
| | Bleed 2 | 116014 | 55555 | 205073 | 53970 |
| | Bleed 3 | 0 | 197337 | 678738 | 221840 |

TABLE 20
Letx Neutralizing Antibody Titers

Group

| | A | B | C | D |
|---|---|---|---|---|
| Mean | 184 | 226 | 160 | 149 |
| Std. Dev. | 217 | 372 | 178 | 390 |

Group

| | E | F | G | H |
|---|---|---|---|---|
| Mean | 92 | 86 | 211 | 35 |
| Std. Dev | 111 | 89 | 212 | 46 |

### Example 15

### Immunization of Non-Human Primates Using Codon-Optimized B. anthracis DNA Vaccines

Three groups of cynomologous macaques (M. fasicularis), containing three monkeys in each group, were used in this experiment. The animals were immunized unilaterally, intramuscularly, in the deltoid muscle with a Bioinjector device. Varying amounts of purified VR6292 (PA83Δfurin) plasmid DNA (prepared as described in the plasmid DNA purification section prior to Example 1) formulated with Vaxfectin™, in a 4:1 molar ratio of plasmid DNA to lipid, was used in all inoculations in this study. All animals received injections at month 0, 1 month, and 2 months. Group 1 received 20 μg of plasmid DNA at each inoculation. Group 2 received 100 μg of plasmid DNA at each inoculation. Group 3 received 200 μg of plasmid DNA at each inoculation.

The monkeys were bled for serum prior to each DNA immunization at month 0 (Bleed 1), month 1 (Bleed 2), month 2 (Bleed 3) and at four weeks after the last injection (Bleed 4). Anti-PA IgG antibody titers and neutralization of lethal toxin (Letx) titers were performed as described in Example 9. The antibody titers and neutralization results for each group of animals are shown in Tables 21, 22 and 23.

2 out of 3 animals in Group 1 generated an anti-PA IgG titer. One of the animals generated a sizable titer (20,000) after three injections. This titer is comparable to the titers of the animals in groups receiving higher doses of plasmid DNA (Groups 2 and 3). None of the animals in Group 1 generated any measurable neutralization activity of Letx at the lowest dilutions tested (serum diluted 1:20).

The animals in Groups 2 and 3 generated similar immune responses to the inoculations. All monkeys in both groups developed anti-PA IgG titers. Letx neutralization titers were generated in 2 out of 3 monkeys in both groups. The remaining animal in each group had measurable neutralization activity, but below the level needed to score a titer.

TABLE 21
Group 1 - Anti-PA IgG and LetX Neutralizing Titers

| | Animal # | | |
|---|---|---|---|
| | 1001 | 1002 | 1003 |
| Bleed 2 | 80 | 160 | 640 |
| Bleed 3 | 80 | 640 | 10240 |
| Bleed 4 | | 2560 | 20480 |
| Letx Neutralizing | | | |
| Titer | | | |
| Bleed 3 | 0 | 0 | 0 |
| Bleed 4 | 0 | 0 | 0 |

TABLE 22
Group 2 - Anti-PA IgG and LetX Neutralizing Titers

| Animal # | | 2001 | 2002 | 2003 |
|---|---|---|---|---|
| | Anti-PA IgG | | | |
| | Bleed 2 | 640 | 10240 | 5120 |
| | Bleed 3 | 10240 | 20480 | 40960 |
| | Bleed 4 | 40960 | 40960 | 81920 |
| | Letx | | | |
| | Neutralization | | | |
| | Titer | | | |
| | Bleed 3 | 0 | *** | *** |
| | Bleed 4 | *** | 40 | 80 |

*** denotes a detectable low level of neutralization activity

TABLE 23
Group 3 - Anti-PA IgG and LetX Neutralizing Titers

| Animal # | | 3001 | 3002 | 3003 |
|---|---|---|---|---|
| | Anti-PA IgG | | | |
| | Bleed 2 | 640 | 320 | 640 |
| | Bleed 3 | 5120 | 20480 | 10240 |
| | Bleed 4 | 20480 | 20480 | 81920 |
| | Letx Neutralizing | | | |
| | Titer | | | |
| | Bleed 3 | 0 | 40 | *** |
| | Bleed 4 | *** | 160 | 80 |

*** denotes a detectable low level of neutralization activity

### Example 16

### Immunization Challenge of Rabbits Using Codon-Optimized B. anthracis DNA Vaccines

a) Long-Term Immune Response in DNA Immunized Rabbits

10 rabbits immunized, as described in Example 12, Group D (Immunized three times with VR6292), were followed long-term for anti-PA antibody titer, LetX neutralization titer and protective immune response to an anthrax spore challenge. Anti-PA IgG antibody titers and LetX neutralization titers were performed as described in Example 9. The results of the titers and neutralization assays are shown in Table 24. Rabbits were bled twelve times on the weeks indicated in Table 24.

On week 39 of the experiment, rabbits were challenged by aerosol administration of B. anthracis (Ames strain) spores by standard methods as described in Example 13. All rabbits survived. Control animals that were not vaccinated did not survive challenge.

TABLE 24

| | | |
|---|---|---|
| Week | | |
| (post first injection) | Geometric Mean | Std. Dev. |

Anti-PA IgG Serum Antibody Titer

| | | |
|---|---|---|
| 2 | 20480 | 22744 |
| 6 | 2622775 | 1635799 |
| 10 | 12909485 | 6079129 |
| 14 | 6456057 | 8244196 |
| 18 | 4565122 | 7496131 |
| 22 | 2810448 | 2931335 |
| 26 | 1311120 | 1508050 |
| 30 | 1311120 | 1508050 |
| 34 | 1405367 | 888676 |
| 39 | 1064744 | 1475391 |
| 40 | 1505928 | 1809833 |
| 42 | 2129704 | 1865167 |

Letx Neutralization Titer

| | | |
|---|---|---|
| 6 | 1576 | 843 |
| 10 | 4457 | 2956 |
| 14 | 2560 | 1602 |
| 18 | 1372 | 1441 |
| 22 | 1194 | 1455 |
| 26 | 970 | 607 |
| 30 | 905 | 641 |
| 35 | 905 | 641 |
| 40 | 844 | 843 |
| 41 | 1040 | 911 |
| 43 | 1194 | 955 |

b) Rabbit Immunization Dosing with Intended Human Vaccine Product

Sixty New Zealand White rabbits (30 males and 30 females), approximately 10-12 weeks old, were used for this study. Ten animals per sex were injected with the formulations described below. The plasmids were formulation with DMRIE/DOPE in a 4:1 DNA to lipid mass ratio in PBS, as described in Example 8b.
Group 1: 1.0 ml of PBS
Group 2: 0.1 mg of plasmid VR-6292 and 0.1 mg of plasmid VR 62952.
Group 3: 1.0 mg of plasmids VR-6292 and 1.0 mg VR-62952.
   All animals in the study received unilateral intramuscular injections into the vastus lateralus muscle at 0, 2, 4 and 8 weeks.

Serum samples were taken from all study animals once during the pretreatment period and once during weeks 2, 4, 6, 8, 10 and 12. Anti-PA and LF antibody titers and Letx neutralizing antibody titers were evaluated using serum samples taken prior to immunization and at 8 weeks prior to the fourth DNA immunization. All immunological assays were performed as described in Example 9. Anti-PF and LF antibody titers and Letx neutralizing antibody results for the bleeds taken at week 8 are shown in Tables 25 and 26.

TABLE 25
Anti-PA and Anti-LF Antibody Titers (Geometric Mean)

Group

| | 2 | 3 |
|---|---|---|
| Anti-PA | 163840 | 514211 |
| Anti-LF | 163840 | 678540 |

Std. Dev.

| | | |
|---|---|---|
| Anti-PA | 230686 | 595754 |
| Anti-LF | 386254 | 730464 |

TABLE 26
Letx Neutralization Titers (Geometric Mean)

Group

| | 2 | 3 |
|---|---|---|
| Letx titer | 889 | 2840 |
| Std Dev | 510 | 1518 |

c) Post Challenge Immune Responses in Aerosolized Spore Challenged Rabbits.

Six groups of rabbits, with 10 individuals in each group, were immunized as described for Groups A-C, G, H and K in Example 12. 39 weeks after the last immunization, these rabbits were challenged with anthrax aerosolized spores, as described in Example 13. Control animals that had not been immunized were also challenged as described in Example 13. No control animal survived the challenge.

At one day prior to challenge, and at 7 and 21 days post challenge, animals were bled for serum. Anti-PA and LF IgG antibody and LetX neutralizing titers were performed as described in Example 9. It should be noted that except as described below, immunized animals had developed protective immunity since they survived challenge.

The immune responses post challenge could be divided into two groups: rabbits that showed no increase in immune response after challenge (lack of boosting) and rabbits that were boosted in their response to PA and/or LF after spore challenge.

All rabbits immunized as described for Groups A-C, in Example 12 (immunized with VR6292 or VR6292+VR62952, three times), demonstrated a lack of boosting. Two rabbits immunized as described for Group G, in Example 12 (immunized with VR6292 twice), had the lowest anti-PA titers pre-challenge and demonstrated a small post-challenge boost in anti-PA titer and the generation of an anti-LF response.

Several rabbits immunized as described for Group K, in Example 12 (immunized with VR62952 (LF[I-III])), did not survive anthrax spore challenge. The five surviving rabbits all had significant anti-PA titers post challenge. Additionally rabbits immunized as described for Group H in Example 12 (immunized twice with the commercial anthrax vaccine AVA), had no measurable anti-LF response pre-challenge. After challenge all rabbits showed a boosted anti-PA titer and the generation of a strong anti-LF response.

In summary, all rabbits immunized two or three times with plasmids encoding PA or PA+LF generated strong immune responses and were able to survive anthrax spore challenge. Almost all of these rabbits showed a lack of immune response boosting post-challenge, which is consistent with sterilizing immunity. In contrast, rabbits immunized twice with 50 μl of AVA exhibited a strong anti-LF response and a boosted anti-PA titer.

### Example 17

### Mucosal Vaccination and Electrically Assisted Plasmid Delivery

a) Mucosal DNA Vaccination

Plasmid constructs comprising codon-optimized and non-codon-optimized coding regions encoding LF, PA or various fragments, variants or derivatives, as described herein, are delivered to BALB/c mice at 0, 2 and 4 weeks via i.m., intranasal (i.n.), intravenous (i.v.), intravaginal (i.vag.), intrarectal (i.r.) or oral routes. The DNA is delivered unformulated or formulated with the cationic lipids DMRIE/DOPE (DD), DMRIE/Cholesterol or Vaxfectin™. Serum IgG titers against the various LF and PA antigens are measured as described in Example 9, as well as Letx neutralization titers.

b) Electrically-Assisted Plasmid Delivery

In vivo gene delivery may be enhanced through the application of brief electrical pulses to injected tissues, a procedure referred to herein as electrically-assisted plasmid delivery. See, e.g., Aihara, H. & Miyazaki, J. Nat. Biotechnol. 16:867-70 (1998); Mir, L. M. et al., Proc. Natl. Acad. Sci. USA 96:4262-67 (1999); Hartikka, J. et al., Mol. Ther. 4:407-15 (2001); and Mir, L. M. et al.; Rizzuto, G. et al., Hum Gene Ther 11:1891-900 (2000); Widera, G. et al, J. of Immuno. 164: 4635-4640 (2000). The use of electrical pulses for cell electropermeabilization has been used to introduce foreign DNA into prokaryotic and eukaryotic cells in vitro. Cell permeabilization can also be achieved locally, in vivo, using electrodes and optimal electrical parameters that are compatible with cell survival.

The electroporation procedure can be performed with various electroporation devices. These devices include external plate type electrodes or invasive needle/rod electrodes and can possess two electrodes or multiple electrodes placed in an array. Distances between the plate or needle electrodes can vary depending upon the number of electrodes, size of target area and treatment subject.

The TriGrid needle array, used in examples described herein, is a three electrode array comprising three elongate electrodes in the approximate shape of a geometric triangle. Needle arrays may include single, double, three, four, five, six or more needles arranged in various array formations. The electrodes are connected through conductive cables to a high voltage switching device that is connected to a power supply.

The electrode array is placed into the muscle tissue, around the site of nucleic acid injection, to a depth of approximately 3 mm to 3 cm. The depth of insertion varies depending upon the target tissue and size of patient receiving electroporation. After injection of foreign nucleic acid, such as plasmid DNA, and a period of time sufficient for distribution of the nucleic acid, square wave electrical pulses are applied to the tissue. The amplitude of each pulse ranges from about 100 volts to about 1500 volts, e.g., about 100 volts, about 200 volts, about 300 volts, about 400 volts, about 500 volts, about 600 volts, about 700 volts, about 800 volts, about 900 volts, about 1000 volts, about 1100 volts, about 1200 volts, about 1300 volts, about 1400 volts, or about 1500 volts or about 1-1.5 kV/cm, based on the spacing between electrodes. Each pulse has a duration of about 1 μs to about 1000 μs, e.g., about 1 μs, about 10 μs, about 50 μs, about 100 μs, about 200 μs, about 300 μs, about 400 μs, about 500 μs, about 600 μs, about 700 μs, about 800 μs, about 900 μs, or about 1000 μs, and a pulse frequency on the order of about 1-10 Hz. The polarity of the pulses may be reversed during the electroporation procedure by switching the connectors to the pulse generator. Pulses are repeated multiple times. The electroporation parameters (e.g. voltage amplitude, duration of pulse, number of pulses, depth of electrode insertion and frequency) will vary based on target tissue type, number of electrodes used and distance of electrode spacing, as would be understood by one of ordinary skill in the art.

Immediately after completion of the pulse regimen, subjects receiving electroporation can be optionally treated with membrane stabilizing agents to prolong cell membrane permeability as a result of the electroporation. Examples of membrane stabilizing agents include, but are not limited to, steroids (e.g. dexamethasone, methylprednisone and progesterone), angiotensin II and vitamin E. A single dose of dexamethasone, approximately 0.1 mg per kilogram of body weight, should be sufficient to achieve a beneficial affect.

EAPD techniques such as electroporation can also be used for plasmids contained in liposome formulations. The liposome-plasmid suspension is administered to the animal or patient and the site of injection is treated with a safe but effective electrical field generated, for example, by a TriGrid needle array. The electroporation may aid in plasmid delivery to the cell by destabilizing the liposome bilayer so that membrane fusion between the liposome and the target cellular structure occurs. Electroporation may also aid in plasmid delivery to the cell by triggering the release of the plasmid, in high concentrations, from the liposome at the surface of the target cell so that the plasmid is driven across the cell membrane by a concentration gradient via the pores created in the cell membrane as a result of the electroporation.

Female BALB/c mice aged 8-10 weeks are anesthetized with inhalant isoflurane and maintained under anesthesia for the duration of the electroporation procedure. The legs are shaved prior to treatment. Plasmid constructs comprising codon-optimized and non-codon-optimized coding regions which encode LF, PA or various fragments, variants or derivatives, as described herein, are administered to BALB/c mice (n=10) via unilateral injection in the quadriceps, with 50 μg total of a plasmid DNA per mouse, using an 0.3 cc insulin syringe and a 26 gauge, ½ length needle fitted with a plastic collar to regulate injection depth. Approximately one minute after injection, electrodes are applied. Modified caliper electrodes are used to apply the electrical pulse. See Hartikka J. et al. Mol Ther 188:407-415 (2001). The caliper electrode plates are coated with conductivity gel and applied to the sides of the injected muscle before closing to a gap of 3 mm for administration of pulses. EAPD is applied using a square pulse type at 1-10 Hz with a field strength of 100-500 V/cm, 1-10 pulses, of 10-100 ms each.

Mice are vaccinated ±EAPD at 0, 2 and 4 weeks. As endpoints, serum IgG titers against the various LF and PA antigens are measured as described in Example 9, as well as Letx neutralization titers.

Rabbits (n=3) are given bilateral injections in the quadriceps muscle with plasmid constructs comprising codon-optimized and non-codon-optimized coding regions which encode LF, PA or various fragments, variants or derivatives, as described herein. The implantation area is shaved and the TriGrid electrode array is implanted into the target region of the muscle. 3.0 mg of plasmid DNA is administered per dose through the injection port of the electrode array. An injection collet is used to control the depth of injection. Electroporation begins approximately one minute after injection of the plasmid DNA is complete. Electroporation is administered with a TriGrid needle array, with electrodes evenly spaced 7 mm apart, using an Ichor TGP-2 pulse generator. The array is inserted into the target muscle to a depth of about 1 to 2 cm. 4-8 pulses are administered. Each pulse has a duration of about 50-100 μs, an amplitude of about 1-1.2 kV/cm and a pulse frequency of 1 Hz. The injection and electroporation may be repeated.

Sera are collected from vaccinated rabbits at various time point. As endpoints, serum IgG titers against the various LF and PA antigens are measured as described in Example 9, as well as Letx neutralization titers.

To test the effect of electroporation on therapeutic protein expression in non-human primates, male or female cynomonologous macques are given either 2 or 6 i.m. injections of plasmid constructs comprising codon-optimized and non-codon-optimized coding regions which encode LF, PA or various fragments, variants or derivatives, as described herein, (0.1 to 10 mg DNA total per animal). Target muscle groups include, but are not limited to, bilateral rectus fermoris, cranial tibialis, biceps, gastrocenemius or deltoid muscles. The target area is shaved and a needle array, comprising between 4 and 10 electrodes, spaced between 0.5-1.5 cm apart, is implanted into the target muscle. Once injections are complete, a sequence of brief electrical pulses are applied to the electrodes implanted in the target muscle using an Ichor TGP-2 pulse generator. The pulses have an amplitude of approximately 120-200V. The pulse sequence is completed within one second. During this time, the target muscle may make brief contractions or twitches. The injection and electroporation may be repeated.

Sera are collected from vaccinated monkeys at various time points. As endpoints, serum IgG titers against the various LF and PA antigens are measured as described in Example 9, as well as Letx neutralization titers.

The present invention is not to be limited in scope by the specific embodiments described which are intended as single illustrations of individual aspects of the invention, and any compositions or methods which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims.

All publications and patent applications mentioned in this specification are herein incorporated by reference to the same extent as if each individual publication or patent application was specifically and individually indicated to be incorporated by reference.

1-214. (canceled)

215. A method to prevent anthrax infection in a vertebrate comprising: administering to a vertebrate in need thereof a composition comprising a carrier, (±)-N-(3-aminopropyl)-N,N-dimethyl-2,3-bis(syn-9-tetradecenyloxy)-1-propanaminium bromide (GAP-DMORIE), a co-lipid and an isolated polynucleotide comprising a nucleic acid fragment which encodes a polypeptide at least 97% identical to amino acids 199 to 764 of SEQ ID NO:4,
wherein said composition elicits an immune response to said polypeptide;
and wherein said nucleic acid fragment is a variant fragment of an optimized coding region for the polypeptide of SEQ ID NO:4;
wherein about 11 of the 24 phenylalanine codons in said coding region are TTT and about 13 of said phenylalanine codons are TTC;
wherein about 5 of the 62 leucine codons in said coding region are TTA, about 8 of said leucine codons are TTG, about 8 of said leucine codons are CTT, about 12 of said leucine codons are CTC, about 4 of said leucine codons are CTA, and about 25 of said leucine codons are CTG;
wherein about 20 of the 57 isoleucine codons in said coding region are ATT, about 28 of said isoleucine codons are ATC, and about 9 of said isoleucine codons are ATA;
wherein the 10 methionine codons in said coding region are ATG;
wherein about 8 of the 43 valine codons in said coding region are GTT, about 10 of said valine codons are GTG, about 5 of said valine codons are GTA, and about 20 of said valine codons are GTG;
wherein about 13 of the 72 serine codons in said coding region are TCT, about 16 of said serine codons are TCC, about 11 of said serine codons are TCA, about 4 of said serine codons are TCG, about 11 of said serine codons are AGT, and about 17 of said serine codons are AGC;
wherein about 8 of the 29 proline codons in said coding region are CCT, about 10 of said proline codons are CCC, about 8 of said proline codons are CCA, and about 3 of said proline codons are CCG;
wherein about 14 of the 58 threonine codons in said coding region are ACT, about 21 of said threonine codons are ACC, about 16 of said threonine codons are ACA, and about 7 of said threonine codons are ACG;
wherein about 11 of the 41 alanine codons in said coding region are GGT, about 17 of said alanine codons are GCC, about 9 of said alanine codons are GCA, and about 4 of said alanine codons are GCG;
wherein about 12 of the 28 tyrosine codons in said coding region are TAT and about 16 of said tyrosine codons are TAC;
wherein about 4 of the 10 histidine codons in said coding region are CAT and about 6 of said histidine codons are CAC;
wherein about 8 of the 31 glutamine codons in said coding region are CAA and about 23 of said glutamine codons are CAG;
wherein about 32 of the 69 asparagine codons in said coding region are AAT and about 37 of said asparagine codons are AAC;
wherein about 25 of the 60 lysine codons in said coding region are AAA and about 35 of said lysine codons are AAG;
wherein about 22 of the 47 aspartic acid codons in said coding region are GAT and about 25 of said aspartic acid codons are GAC;
wherein about 21 of the 51 glutamic acid codons in said coding region are GAA and about 30 of said glutamic acid codons are GAG;
wherein the 7 tryptophan codons in said coding region are TGG;
wherein about 2 of the 29 arginine codons in said coding region are CGT, about 6 of said arginine codons are CGC, about 3 of said arginine codons are CGA, about 6 of said arginine codons are CGG, about 6 of said arginine codons are AGA, and about 6 of said arginine codons are AGG; and
wherein about 6 of the 36 glycine codons in said coding region are GGT, about 12 of said glycine codons are GGC, about 9 of said glycine codons are GGA, and about 9 of said glycine codons are GGG.

216. The method of claim 215, wherein the amino acids of said polypeptide corresponding to amino acids 342 and 343 of SEQ ID NO:4 have been deleted.

217. The method of claim 215, wherein said polypeptide comprises amino acids 199 to 764 of SEQ ID NO:4.

218. The method of claim 215, wherein said nucleic acid fragment encodes a polypeptide at least 97% identical to amino acids 30 to 764 of SEQ ID NO:4.

219. The method of claim 217, wherein said polypeptide comprises amino acids 30 to 764 of SEQ ID NO:4.

220. The method of claim 218, wherein the amino acids of said polypeptide corresponding to amino acids 192 to 197 of SEQ ID NO:4 have been deleted.

221. The method of claim 220, wherein said polypeptide is SEQ ID NO:8.

222. The method of claim 215, wherein said nucleic acid fragment is ligated to a heterologous nucleic acid.

223. The method of claim 222, wherein said heterologous nucleic acid encodes a heterologous polypeptide fused to the polypeptide encoded by said nucleic acid fragment.

224. The method of claim 223, wherein said heterologous polypeptide is a secretory signal peptide.

225. The method of claim 224, wherein said signal peptide is a human tissue plasminogen activator (hTPA) signal peptide.

226. The method of claim 215, wherein said co-lipid is selected from the group consisting of:
1,2-dioleoyl-sn-glycero-3-phosphoethanolamine (DOPE),
1,2-diphytanoyl-sn-glycero-3-phosphoethanolamine (DPyPE), and
1,2-dimyristoyl-glycer-3-phosphoethanolamine (DMPE).

227. The method of claim 226, wherein said co-lipid is DPyPE.

228. The method of claim 227, wherein said GAP-DMORIE and DPyPE are in a 1:1 molar ratio.

229. The method of claim 228, wherein said polypeptide is SEQ ID NO:8.

230. The method of claim 215, wherein said composition further comprises a nucleic acid molecule which encodes a polypeptide comprising an amino acid sequence selected from the group consisting of: amino acids 30 to 764 of SEQ ID NO:4, SEQ ID NO:6; SEQ ID NO:8; and a combination of two or more of said amino acid sequences.

231. A method to treat anthrax infection in a vertebrate comprising: administering to a vertebrate in need thereof a composition comprising a carrier, (±)-N-(3-aminopropyl)-N,N-dimethyl-2,3-bis(syn-9-tetradecenyloxy)-1-propanaminium bromide (GAP-DMORIE), a co-lipid and an isolated polynucleotide comprising a nucleic acid fragment which encodes a polypeptide at least 97% identical to amino acids 199 to 764 of SEQ ID NO:4,
wherein said composition elicits an immune response to said polypeptide;
and wherein said nucleic acid fragment is a variant fragment of an optimized coding region for the polypeptide of SEQ ID NO:4;
wherein about 11 of the 24 phenylalanine codons in said coding region are TTT and about 13 of said phenylalanine codons are TTC;
wherein about 5 of the 62 leucine codons in said coding region are TTA, about 8 of said leucine codons are TTG, about 8 of said leucine codons are CTT, about 12 of said leucine codons are CTC, about 4 of said leucine codons are CTA, and about 25 of said leucine codons are CTG;
wherein about 20 of the 57 isoleucine codons in said coding region are ATT, about 28 of said isoleucine codons are ATC, and about 9 of said isoleucine codons are ATA;
wherein the 10 methionine codons in said coding region are ATG;
wherein about 8 of the 43 valine codons in said coding region are GTT, about 10 of said valine codons are GTG, about 5 of said valine codons are GTA, and about 20 of said valine codons are GTG;
wherein about 13 of the 72 serine codons in said coding region are TCT, about 16 of said serine codons are TCC, about 11 of said serine codons are TCA, about 4 of said serine codons are TCG, about 11 of said serine codons are AGT, and about 17 of said serine codons are AGC;
wherein about 8 of the 29 proline codons in said coding region are CCT, about 10 of said proline codons are CCC, about 8 of said proline codons are CCA, and about 3 of said proline codons are CCG;
wherein about 14 of the 58 threonine codons in said coding region are ACT, about 21 of said threonine codons are ACC, about 16 of said threonine codons are ACA, and about 7 of said threonine codons are ACG;
wherein about 11 of the 41 alanine codons in said coding region are GGT, about 17 of said alanine codons are GCC, about 9 of said alanine codons are GCA, and about 4 of said alanine codons are GCG;
wherein about 12 of the 28 tyrosine codons in said coding region are TAT and about 16 of said tyrosine codons are TAC;
wherein about 4 of the 10 histidine codons in said coding region are CAT and about 6 of said histidine codons are CAC;
wherein about 8 of the 31 glutamine codons in said coding region are CAA and about 23 of said glutamine codons are CAG;
wherein about 32 of the 69 asparagine codons in said coding region are AAT and about 37 of said asparagine codons are AAC;
wherein about 25 of the 60 lysine codons in said coding region are AAA and about 35 of said lysine codons are AAG;
wherein about 22 of the 47 aspartic acid codons in said coding region are GAT and about 25 of said aspartic acid codons are GAC;
wherein about 21 of the 51 glutamic acid codons in said coding region are GAA and about 30 of said glutamic acid codons are GAG;
wherein the 7 tryptophan codons in said coding region are TGG;
wherein about 2 of the 29 arginine codons in said coding region are CGT, about 6 of said arginine codons are CGC, about 3 of said arginine codons are CGA, about 6 of said arginine codons are CGG, about 6 of said arginine codons are AGA, and about 6 of said arginine codons are AGG; and
wherein about 6 of the 36 glycine codons in said coding region are GGT, about 12 of said glycine codons are GGC, about 9 of said glycine codons are GGA, and about 9 of said glycine codons are GGG.

232. The method of claim 231, wherein the amino acids of said polypeptide corresponding to amino acids 342 and 343 of SEQ ID NO:4 have been deleted.

233. The method of claim 231, wherein said polypeptide comprises amino acids 199 to 764 of SEQ ID NO:4.

234. The method of claim 231, wherein said nucleic acid fragment encodes a polypeptide at least 97% identical to amino acids 30 to 764 of SEQ ID NO:4.

235. The method of claim 233, wherein said polypeptide comprises amino acids 30 to 764 of SEQ ID NO:4.

236. The method of claim 234, wherein the amino acids of said polypeptide corresponding to amino acids 192 to 197 of SEQ ID NO:4 have been deleted.

237. The method of claim 236, wherein said polypeptide is SEQ ID NO:8.

238. The method of claim 231, wherein said nucleic acid fragment is ligated to a heterologous nucleic acid.

239. The method of claim 238, wherein said heterologous nucleic acid encodes a heterologous polypeptide fused to the polypeptide encoded by said nucleic acid fragment.

240. The method of claim 239, wherein said heterologous polypeptide is a secretory signal peptide.

241. The polynucleotide of claim 240, wherein said signal peptide is a human tissue plasminogen activator (hTPA) signal peptide.

242. The method of claim 231, wherein said co-lipid is selected from the group consisting of:
1,2-dioleoyl-sn-glycero-3-phosphoethanolamine (DOPE),
1,2-diphytanoyl-sn-glycero-3-phosphoethanolamine (DPyPE), and
1,2-dimyristoyl-glycer-3-phosphoethanolamine (DMPE).

243. The method of claim 242, wherein said co-lipid is DPyPE.

244. The method of claim 231, wherein said composition further comprises a nucleic acid molecule which encodes a polypeptide comprising an amino acid sequence selected from the group consisting of: amino acids 30 to 764 of SEQ ID NO:4, SEQ ID NO:6, SEQ ID NO:8 and a combination of two or more of said amino acid sequences.

245. A method to prevent anthrax infection in a vertebrate comprising: administering to a vertebrate in need thereof a composition comprising a carrier, (±)-N-(2-hydroxyethyl)-N,N-dimethyl-2,3-bis(tetradecyloxy)-1-propanaminium bromide (DMRIE), a co-lipid and an isolated polynucleotide comprising a nucleic acid fragment which encodes a polypeptide at least 97% identical to amino acids 30 to 764 of SEQ ID NO:4;
wherein said composition elicits an immune response to said polypeptide;
and wherein said nucleic acid fragment is a variant fragment of an optimized coding region for the polypeptide of SEQ ID NO:4;
wherein about 11 of the 24 phenylalanine codons in said coding region are TTT and about 13 of said phenylalanine codons are TTC;
wherein about 5 of the 62 leucine codons in said coding region are TTA, about 8 of said leucine codons are TTG, about 8 of said leucine codons are CTT, about 12 of said leucine codons are CTC, about 4 of said leucine codons are CTA, and about 25 of said leucine codons are CTG;
wherein about 20 of the 57 isoleucine codons in said coding region are ATT, about 28 of said isoleucine codons are ATC, and about 9 of said isoleucine codons are ATA;
wherein the 10 methionine codons in said coding region are ATG;
wherein about 8 of the 43 valine codons in said coding region are GTT, about 10 of said valine codons are GTG, about 5 of said valine codons are GTA, and about 20 of said valine codons are GTG;
wherein about 13 of the 72 serine codons in said coding region are TCT, about 16 of said serine codons are TCC, about 11 of said serine codons are TCA, about 4 of said serine codons are TCG, about 11 of said serine codons are AGT, and about 17 of said serine codons are AGC;
wherein about 8 of the 29 proline codons in said coding region are CCT, about 10 of said proline codons are CCC, about 8 of said proline codons are CCA, and about 3 of said proline codons are CCG;
wherein about 14 of the 58 threonine codons in said coding region are ACT, about 21 of said threonine codons are ACC, about 16 of said threonine codons are ACA, and about 7 of said threonine codons are ACG;
wherein about 11 of the 41 alanine codons in said coding region are GGT, about 17 of said alanine codons are GCC, about 9 of said alanine codons are GCA, and about 4 of said alanine codons are GCG;
wherein about 12 of the 28 tyrosine codons in said coding region are TAT and about 16 of said tyrosine codons are TAC;
wherein about 4 of the 10 histidine codons in said coding region are CAT and about 6 of said histidine codons are CAC;
wherein about 8 of the 31 glutamine codons in said coding region are CAA and about 23 of said glutamine codons are CAG;
wherein about 32 of the 69 asparagine codons in said coding region are AAT and about 37 of said asparagine codons are AAC;
wherein about 25 of the 60 lysine codons in said coding region are AAA and about 35 of said lysine codons are AAG;
wherein about 22 of the 47 aspartic acid codons in said coding region are GAT and about 25 of said aspartic acid codons are GAC;
wherein about 21 of the 51 glutamic acid codons in said coding region are GAA and about 30 of said glutamic acid codons are GAG;
wherein the 7 tryptophan codons in said coding region are TGG;
wherein about 2 of the 29 arginine codons in said coding region are CGT, about 6 of said arginine codons are CGC, about 3 of said arginine codons are CGA, about 6 of said arginine codons are CGG, about 6 of said arginine codons are AGA, and about 6 of said arginine codons are AGG; and
wherein about 6 of the 36 glycine codons in said coding region are GGT, about 12 of said glycine codons are GGC, about 9 of said glycine codons are GGA, and about 9 of said glycine codons are GGG.

246. The method of claim 245, wherein the amino acids of said polypeptide corresponding to amino acids 342 and 343 of SEQ ID NO:4 have been deleted.

247. The method of claim 245, wherein said polypeptide comprises amino acids 199 to 764 of SEQ ID NO:4.

248. The method of claim 245, wherein said nucleic acid fragment encodes a polypeptide at least 97% identical to amino acids 30 to 764 of SEQ ID NO:4.

249. The method of claim 247, wherein said polypeptide comprises amino acids 30 to 764 of SEQ ID NO:4.

250. The method of claim 248, wherein said the amino acids of said polypeptide corresponding to amino acids 192 to 197 of SEQ ID NO:4 have been deleted.

251. The method of claim 250, wherein said polypeptide is SEQ ID NO:8.

252. The method of claim 245, wherein said nucleic acid fragment is ligated to a heterologous nucleic acid.

253. The method of claim 252, wherein said heterologous nucleic acid encodes a heterologous polypeptide fused to the polypeptide encoded by said nucleic acid fragment.

254. The method of claim 253, wherein said heterologous polypeptide is a secretory signal peptide.

255. The method of claim 254, wherein said signal peptide is a human tissue plasminogen activator (hTPA) signal peptide.

256. The method of claim 245, wherein said co-lipid is selected from the group consisting of:
1,2-dioleoyl-sn-glycero-3-phosphoethanolamine (DOPE),
1,2-diphytanoyl-sn-glycero-3-phosphoethanolamine (DPyPE), and
1,2-dimyristoyl-glycer-3-phosphoethanolamine (DMPE).

257. The method of claim 256, wherein said co-lipid is 1,2-dioleoyl-sn-glycero-3-phosphoethanolamine (DOPE).

258. The method of claim 257, wherein said DMRIE and DOPE are in a 1:1 molar ratio.

259. The method of claim 258, wherein said polypeptide is SEQ ID NO:8.

260. The method of claim 245, wherein said composition further comprises a nucleic acid molecule which encodes a polypeptide comprising an amino acid sequence selected from the group consisting of: amino acids 30-764 of SEQ ID NO:4, SEQ ID NO:6, SEQ ID NO:8 and a combination of two or more of said amino acid sequences.

261. A method to treat anthrax infection in a vertebrate comprising: administering to a vertebrate in need thereof a composition comprising a carrier, (±)-N-(2-hydroxyethyl)-N,N-dimethyl-2,3-bis(tetradecyloxy)-1-propanaminium bromide (DMRIE), a co-lipid and an isolated polynucleotide comprising a nucleic acid fragment which encodes a polypeptide at least 97% identical to amino acids 30 to 764 of SEQ ID NO:4;
wherein said composition elicits an immune response to said polypeptide;
and wherein said nucleic acid fragment is a variant fragment of an optimized coding region for the polypeptide of SEQ ID NO:4;
wherein about 11 of the 24 phenylalanine codons in said coding region are TTT and about 13 of said phenylalanine codons are TTC;
wherein about 5 of the 62 leucine codons in said coding region are TTA, about 8 of said leucine codons are TTG, about 8 of said leucine codons are CTT, about 12 of said leucine codons are CTC, about 4 of said leucine codons are CTA, and about 25 of said leucine codons are CTG;
wherein about 20 of the 57 isoleucine codons in said coding region are ATT, about 28 of said isoleucine codons are ATC, and about 9 of said isoleucine codons are ATA;
wherein the 10 methionine codons in said coding region are ATG;
wherein about 8 of the 43 valine codons in said coding region are GTT, about 10 of said valine codons are GTG, about 5 of said valine codons are GTA, and about 20 of said valine codons are GTG;
wherein about 13 of the 72 serine codons in said coding region are TCT, about 16 of said serine codons are TCC, about 11 of said serine codons are TCA, about 4 of said serine codons are TCG, about 11 of said serine codons are AGT, and about 17 of said serine codons are AGC;
wherein about 8 of the 29 proline codons in said coding region are CCT, about 10 of said proline codons are CCC, about 8 of said proline codons are CCA, and about 3 of said proline codons are CCG;
wherein about 14 of the 58 threonine codons in said coding region are ACT, about 21 of said threonine codons are ACC, about 16 of said threonine codons are ACA, and about 7 of said threonine codons are ACG;
wherein about 11 of the 41 alanine codons in said coding region are GGT, about 17 of said alanine codons are GCC, about 9 of said alanine codons are GCA, and about 4 of said alanine codons are GCG;
wherein about 12 of the 28 tyrosine codons in said coding region are TAT and about 16 of said tyrosine codons are TAC;
wherein about 4 of the 10 histidine codons in said coding region are CAT and about 6 of said histidine codons are CAC;
wherein about 8 of the 31 glutamine codons in said coding region are CAA and about 23 of said glutamine codons are CAG;
wherein about 32 of the 69 asparagine codons in said coding region are AAT and about 37 of said asparagine codons are AAC;
wherein about 25 of the 60 lysine codons in said coding region are AAA and about 35 of said lysine codons are AAG;
wherein about 22 of the 47 aspartic acid codons in said coding region are GAT and about 25 of said aspartic acid codons are GAC;
wherein about 21 of the 51 glutamic acid codons in said coding region are GAA and about 30 of said glutamic acid codons are GAG;
wherein the 7 tryptophan codons in said coding region are TGG;
wherein about 2 of the 29 arginine codons in said coding region are CGT, about 6 of said arginine codons are CGC, about 3 of said arginine codons are CGA, about 6 of said arginine codons are CGG, about 6 of said arginine codons are AGA, and about 6 of said arginine codons are AGG; and
wherein about 6 of the 36 glycine codons in said coding region are GGT, about 12 of said glycine codons are GGC, about 9 of said glycine codons are GGA, and about 9 of said glycine codons are GGG.

262. The method of claim 261, wherein the amino acids of said polypeptide corresponding to amino acids 342 and 343 of SEQ ID NO:4 have been deleted.

263. The method of claim 261, wherein said polypeptide comprises amino acids 199 to 764 of SEQ ID NO:4.

264. The method of claim 261, wherein said nucleic acid fragment encodes a polypeptide at least 97% identical to amino acids 30 to 764 of SEQ ID NO:4.

265. The method of claim 263, wherein said polypeptide comprises amino acids 30 to 764 of SEQ ID NO:4.

266. The method of claim 264, wherein the amino acids of said polypeptide corresponding to amino acids 192 to 197 of SEQ ID NO:4 have been deleted.

267. The method of claim 266, wherein said polypeptide is SEQ ID NO:8.

268. The method of claim 261, wherein said nucleic acid fragment is ligated to a heterologous nucleic acid.

269. The method of claim 268, wherein said heterologous nucleic acid encodes a heterologous polypeptide fused to the polypeptide encoded by said nucleic acid fragment.

270. The method of claim 269, wherein said heterologous polypeptide is a secretory signal peptide.

271. The method of claim 270, wherein said signal peptide is a human tissue plasminogen activator (hTPA) signal peptide.

272. The method of claim 261, wherein said co-lipid is selected from the group consisting of:
1,2-dioleoyl-sn-glycero-3-phosphoethanolamine (DOPE),
1,2-diphytanoyl-sn-glycero-3-phosphoethanolamine (DPyPE), and
1,2-dimyristoyl-glycer-3-phosphoethanolamine (DMPE).

273. The method of claim 272, wherein said co-lipid is DPyPE.

274. The method of claim 261, wherein said composition further comprises a nucleic acid molecule which encodes a polypeptide comprising an amino acid sequence selected from the group consisting of: amino acids 30 to 764 of SEQ ID NO:4, SEQ ID NO:6, SEQ ID NO:8 and a combination of two or more of said amino acid sequences.

275. A composition comprising a carrier, a lipid selected from the group consisting of: (±)-N-(3-aminopropyl)-N,N-dimethyl-2,3-bis(syn-9-tetradecenyloxy)-1-propanaminium bromide (GAP-DMORIE), (±)-N-(2-hydroxyethyl)-N,N-dimethyl-2,3-bis(tetradecyloxy)-1-propanaminium bromide (DMRIE) and a combination thereof, a co-lipid, and an isolated polynucleotide comprising a nucleic acid fragment which encodes a polypeptide at least 97% identical to amino acids 199 to 764 of SEQ ID NO:4,
wherein said composition elicits an immune response to said polypeptide;
and wherein said nucleic acid fragment is a variant fragment of an optimized coding region for the polypeptide of SEQ ID NO:4;
wherein about 11 of the 24 phenylalanine codons in said coding region are TTT and about 13 of said phenylalanine codons are TTC;
wherein about 5 of the 62 leucine codons in said coding region are TTA, about 8 of said leucine codons are TTG, about 8 of said leucine codons are CTT, about 12 of said leucine codons are CTC, about 4 of said leucine codons are CTA, and about 25 of said leucine codons are CTG;
wherein about 20 of the 57 isoleucine codons in said coding region are ATT, about 28 of said isoleucine codons are ATC, and about 9 of said isoleucine codons are ATA;
wherein the 10 methionine codons in said coding region are ATG;
wherein about 8 of the 43 valine codons in said coding region are GTT, about 10 of said valine codons are GTG, about 5 of said valine codons are GTA, and about 20 of said valine codons are GTG;
wherein about 13 of the 72 serine codons in said coding region are TCT, about 16 of said serine codons are TCC, about 11 of said serine codons are TCA, about 4 of said serine codons are TCG, about 11 of said serine codons are AGT, and about 17 of said serine codons are AGC;
wherein about 8 of the 29 proline codons in said coding region are CCT, about 10 of said proline codons are CCC, about 8 of said proline codons are CCA, and about 3 of said proline codons are CCG;
wherein about 14 of the 58 threonine codons in said coding region are ACT, about 21 of said threonine codons are ACC, about 16 of said threonine codons are ACA, and about 7 of said threonine codons are ACG;
wherein about 11 of the 41 alanine codons in said coding region are GGT, about 17 of said alanine codons are GCC, about 9 of said alanine codons are GCA, and about 4 of said alanine codons are GCG;
wherein about 12 of the 28 tyrosine codons in said coding region are TAT and about 16 of said tyrosine codons are TAC;
wherein about 4 of the 10 histidine codons in said coding region are CAT and about 6 of said histidine codons are CAC;
wherein about 8 of the 31 glutamine codons in said coding region are CAA and about 23 of said glutamine codons are CAG;
wherein about 32 of the 69 asparagine codons in said coding region are AAT and about 37 of said asparagine codons are AAC;
wherein about 25 of the 60 lysine codons in said coding region are AAA and about 35 of said lysine codons are AAG;
wherein about 22 of the 47 aspartic acid codons in said coding region are GAT and about 25 of said aspartic acid codons are GAC;
wherein about 21 of the 51 glutamic acid codons in said coding region are GAA and about 30 of said glutamic acid codons are GAG;
wherein the 7 tryptophan codons in said coding region are TGG;
wherein about 2 of the 29 arginine codons in said coding region are CGT, about 6 of said arginine codons are CGC, about 3 of said arginine codons are CGA, about 6 of said arginine codons are CGG, about 6 of said arginine codons are AGA, and about 6 of said arginine codons are AGG; and
wherein about 6 of the 36 glycine codons in said coding region are GGT, about 12 of said glycine codons are GGC, about 9 of said glycine codons are GGA, and about 9 of said glycine codons are GGG.

276. The composition of claim 275, wherein the amino acids of said polypeptide corresponding to amino acids 342 and 343 of SEQ ID NO:4 have been deleted.

277. The composition of claim 275, wherein said polypeptide comprises amino acids 199 to 764 of SEQ ID NO:4.

278. The composition of claim 275, wherein said nucleic acid fragment encodes a polypeptide at least 97% identical to amino acids 30 to 764 of SEQ ID NO:4.

279. The composition of claim 277, wherein said polypeptide comprises amino acids 30 to 764 of SEQ ID NO:4.

280. The composition of claim 278, wherein the amino acids of said polypeptide corresponding to amino acids 192 to 197 of SEQ ID NO:4 have been deleted.

281. The composition of claim 280, wherein said polypeptide is SEQ ID NO:8.

282. The composition of claim 275, wherein said nucleic acid fragment is ligated to a heterologous nucleic acid.

283. The composition of claim 282, wherein said heterologous nucleic acid encodes a heterologous polypeptide fused to the polypeptide encoded by said nucleic acid fragment.

284. The composition of claim 283, wherein said heterologous polypeptide is a secretory signal peptide.

285. The composition of claim 284, wherein said signal peptide is a human tissue plasminogen activator (hTPA) signal peptide.

286. The composition of claim 275, wherein said co-lipid is selected from the group consisting of:
1,2-dioleoyl-sn-glycero-3-phosphoethanolamine (DOPE),
1,2-diphytanoyl-sn-glycero-3-phosphoethanolamine (DPyPE), and
1,2-dimyristoyl-glycer-3-phosphoethanolamine (DMPE).

287. The composition of claim 286, wherein said lipid is GAP-DMORIE.

288. The composition of claim 286, wherein said lipid is DMRIE.

289. The composition of claim 287, wherein said co-lipid is DPyPE.

290. The composition of claim 288, wherein said co-lipid is DOPE.

291. The composition of claim 289, wherein said polypeptide is SEQ ID NO:8.

292. The composition of claim 290, wherein said polypeptide is SEQ ID NO:8.


SEQUENCE LISTING (76 sequences)

SEQ ID NO: 1 - DNA, 1788 bp, Artificial Sequence
  Synthetic coding region for Human TPA/B. anthracis antigen fusion protein
  CDS (13)..(1779)
gatatcgccaccatggatgcaatgaagagagggctctgctgtgtgctgctgctgtgtggagcagtcttcgtttcgcccagcagcgctgggccaactgtgcccgacagagacaatgatggaatccctgatagtctagaggttgagggatacacggtagatgtcaagaacaaaaggacttttctctcgccttggatctcaaatatccatgagaagaaggggcttaccaagtacaagtcctcccccgagaagtggtctaccgcttccgatccatatagcgatttcgagaaggtcacaggccggatcgataaaaatgtgtctccagaggctagacaccccctggtagcagcctacccgattgtacacgtggacatggagaacatcattctaagcaaaaacgaggaccagtccacacaaaacactgactccgagacccgcaccatatctaaaaacaccagtacttcaaggacccacacctctgaagtgcacggcaatgcggaagtccatgcatcgtttttcgatattggtggctccgtgtcagccggctttagcaatagcaactcctcgacggttgccattgaccactcactgtcattagcaggtgagaggacttgggctgaaactatgggtctgaataccgccgatacggcccggctcaacgcaaatattcggtacgtcaacacagggactgctcctatatataacgtgctgcctacgacaagtcttgtcctgggcaaaaatcagaccctcgcaaccattaaggcaaaggaaaatcagctgagccagatcctcgcccctaacaactattatccatccaaaaatttagcccccatagccctgaacgcccaggacgacttttcctctacccccataactatgaattacaatcagttcctggagctggaaaagacgaagcagctgagactagacaccgatcaggtgtatggaaacatagcgacatataactttgagaacggccgcgtgcgcgtcgacactgggtcaaactggtctgaagttctgccgcaaattcaagagacaaccgccagaattatctttaatgggaaggacttgaaccttgtcgaacgtagaattgccgccgtgaaccccagtgatccactcgagacgactaaaccggatatgacactgaaagaggctctgaagattgccttcggattcaacgaacctaatggcaatttgcagtatcaggggaaagacatcacagagtttgatttcaatttcgatcagcagacttcccaaaatatcaaaaatcagttggcagagctgaatgccaccaatatctacacggttctcgataaaatcaaacttaacgccaagatgaacatattgattcgagacaaacgcttccactacgaccgcaacaatatagccgtaggcgctgatgagtctgtcgtcaaggaggctcatagggaagttatcaacagcagtactgaagggctgttacttaatatcgacaaggacattcggaagatcctgtccgggtatatcgtggagatcgaggataccgagggcctgaaggaagtcattaacgaccgctatgatatgctgaacatttccagcttacgacaggacggtaagacatttattgactttaaaaagtataacgacaagctacccctgtacatttccaacccaaattacaaagttaatgtgtatgctgtaaccaaggagaacacaatcatcaatccaagcgagaacggcgataccagcacaaatggaatcaaaaagatccttatatttagtaaaaaaggctacgagatcggttgaggatcc

SEQ ID NO: 2 - protein, 589 aa, Artificial Sequence
  Human TPA/ B. anthracis antigen fusion protein
MDAMKRGLCCVLLLCGAVFVSPSSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSETRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 3 - DNA, 4235 bp, Bacillus anthracis
  CDS (1804)..(4098)
aagcttctgtcattcgtaaatttcaaatagaacgtaaatttagacttctcatcattaaaaatgaaaaatcttatctttttgattctattgtatatttttattaaggtgtttaatagttagaaaagacagttgatgctattactccagataaaatatagctaaccataaatttattaaagaaaccttgttgttctaaataatgattttgtggattccggaatagatactggtgagttagctctaattttatagtgatttaactaacaatttataaagcagcataattcaaattttttaattgatttttcctgaagcatagtataaaagagtcaaggtcttctagacttgactcttggaatcattaggaattaacaatatatataatgcgctagacagaatcaaattaaatgcaaaaatgaatattttagtaagagatccatatcattatgataataacggtaatattgtaggggttgatgattcatatttaaaaaacgcatataagcaaatacttaattggtcaagcgatggagtttctttaaatctagatgaagatgtaaatcaagcactatctggatatatgcttcaaataaaaaaaccttcaaaccacctaacaaacagcccagttacaattacattagcaggcaaggacagtggtgttggagaattgtatagagtattatcagatggagcaggattcctggatttcaataagtttgatgaaaattggcgatcattagtagatcctggtgatgatgtttatgtgtatgctgttactaaagaagattttaatgcagttactcgagatgaaaatggtaatatagcgaataaattaaaaaacaccttagttttatcgggtaaaataaaagaaataaacataaaaactacaaatattaatatatttgtagtttttatgtttattatatacctcctattttatattattagtagcacagtttttgcaaatcatgtaattgtatacttatctatgtagaggtatcacaacttatgaatagtgtattttattgaacgttggttagcttggacagttgtatggatatgcatactttataacgtataaaatttcacgcaccacaataaaactaatttaacaaaaacaaaaacacacctaagatcattcagttcttttaataaggagctgcccaccaagctaaacctaaataatctttgtttcacataaggtttttttctaaatatacagtgtaagttattgtgaatttaaccagtatatattaaaaatgttttatgttaacaaattaaattgtaaaacccctcttaagcatagttaagaggggtaggttttaaattttttgttgaaattagaaaaaataataaaaaaacaaacctattttctttcaggttgtttttgggttacaaaacaaaaagaaaacatgtttcaaggtacaataattatggttctttagctttctgtaaaacagccttaatagttggatttatgactattaaagttagtatacagcatacacaatctattgaaggatatttataatgcaattccctaaaaatagttttgtataaccagttcttttatccgaactgatacacgtattttagcataatttttaatgtatcttcaaaaacagcttctgtgtccttttctattaaacatataaattcttttttatgttatatatttataaaagttctgtttaaaaagccaaaaataaataattatctctttttatttatattatattgaaactaaagtttattaatttcaatataatataaatttaattttatacaaaaaggagaacgtatatgaaaaaacgaaaagtgttaataccattaatggcattgtctacgatattagtttcaagcacaggtaatttagaggtgattcaggcagaagttaaacaggagaaccggttattaaatgaatcagaatcaagttcccaggggttactaggatactattttagtgatttgaattttcaagcacccatggtggttacctcttctactacaggggatttatctattcctagttctgagttagaaaatattccatcggaaaaccaatattttcaatctgctatttggtcaggatttatcaaagttaagaagagtgatgaatatacatttgctacttccgctgataatcatgtaacaatgtgggtagatgaccaagaagtgattaataaagcttctaattctaacaaaatcagattagaaaaaggaagattatatcaaataaaaattcaatatcaacgagaaaatcctactgaaaaaggattggatttcaagttgtactggaccgattctcaaaataaaaaagaagtgatttctagtgataacttacaattgccagaattaaaacaaaaatcttcgaactcaagaaaaaagcgaagtacaagtgctggacctacggttccagaccgtgacaatgatggaatccctgattcattagaggtagaaggatatacggttgatgtcaaaaataaaagaacttttctttcaccatggatttctaatattcatgaaaagaaaggattaaccaaatataaatcatctcctgaaaaatggagcacggcttctgatccgtacagtgatttcgaaaaggttacaggacggattgataagaatgtatcaccagaggcaagacacccccttgtggcagcttatccgattgtacatgtagatatggagaatattattctctcaaaaaatgaggatcaatccacacagaatactgatagtgaaacgagaacaataagtaaaaatacttctacaagtaggacacatactagtgaagtacatggaaatgcagaagtgcatgcgtcgttctttgatattggtgggagtgtatctgcaggatttagtaattcgaattcaagtacggtcgcaattgatcattcactatctctagcaggggaaagaacttgggctgaaacaatgggtttaaataccgctgatacagcaagattaaatgccaatattagatatgtaaatactgggacggctccaatctacaacgtgttaccaacgacttcgttagtgttaggaaaaaatcaaacactcgcgacaattaaagctaaggaaaaccaattaagtcaaatacttgcacctaataattattatccttctaaaaacttggcgccaatcgcattaaatgcacaagacgatttcagttctactccaattacaatgaattacaatcaatttcttgagttagaaaaaacgaaacaattaagattagatacggatcaagtatatgggaatatagcaacatacaattttgaaaatggaagagtgagggtggatacaggctcgaactggagtgaagtgttaccgcaaattcaagaaacaactgcacgtatcatttttaatggaaaagatttaaatctggtagaaaggcggatagcggcggttaatcctagtgatccattagaaacgactaaaccggatatgacattaaaagaagcccttaaaatagcatttggatttaacgaaccgaatggaaacttacaatatcaagggaaagacataaccgaatttgattttaatttcgatcaacaaacatctcaaaatatcaagaatcagttagcggaattaaacgcaactaacatatatactgtattagataaaatcaaattaaatgcaaaaatgaatattttaataagagataaacgttttcattatgatagaaataacatagcagttggggcggatgagtcagtagttaaggaggctcatagagaagtaattaattcgtcaacagagggattattgttaaatattgataaggatataagaaaaatattatcaggttatattgtagaaattgaagatactgaagggcttaaagaagttataaatgacagatatgatatgttgaatatttctagtttacggcaagatggaaaaacatttatagattttaaaaaatataatgataaattaccgttatatataagtaatcccaattataaggtaaatgtatatgctgttactaaagaaaacactattattaatcctagtgagaatggggatactagtaccaacgggatcaagaaaattttaatcttttctaaaaaaggctatgagataggataaggtaattctaggtgatttttaaattatctaaaaaacagtaaaattaaaacatactctttttgtaagaaatacaaggagagtatgttttaaacagtaatctaaatcatcataatcctttgagattgtttgtaggatcc

SEQ ID NO: 4 - protein, 764 aa, Bacillus anthracis
MKKRKVLIPLMALSTILVSSTGNLEVIQAEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSETRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 5 - DNA, 1782 bp, Artificial Sequence
  Synthetic coding region for Human TPA/synthetic antigen fusion protein
  CDS (13)..(1773)
gatatcgccaccatggatgcaatgaagagagggctctgctgtgtgctgctgctgtgtggagcagtcttcgtttcgcccagcagcgctgggccaactgtgcccgacagagacaatgatggaatccctgatagtctagaggttgagggatacacggtagatgtcaagaacaaaaggacttttctctcgccttggatctcaaatatccatgagaagaaggggcttaccaagtacaagtcctcccccgagaagtggtctaccgcttccgatccatatagcgatttcgagaaggtcacaggccggatcgataaaaatgtgtctccagaggctagacaccccctggtagcagcctacccgattgtacacgtggacatggagaacatcattctaagcaaaaacgaggaccagtccacacaaaacactgactccgagacccgcaccatatctaaaaacaccagtacttcaaggacccacacctctgaagtgcacggcaatgcggaagtccatgcatcggatattggtggctccgtgtcagccggctttagcaatagcaactcctcgacggttgccattgaccactcactgtcattagcaggtgagaggacttgggctgaaactatgggtctgaataccgccgatacggcccggctcaacgcaaatattcggtacgtcaacacagggactgctcctatatataacgtgctgcctacgacaagtcttgtcctgggcaaaaatcagaccctcgcaaccattaaggcaaaggaaaatcagctgagccagatcctcgcccctaacaactattatccatccaaaaatttagcccccatagccctgaacgcccaggacgacttttcctctacccccataactatgaattacaatcagttcctggagctggaaaagacgaagcagctgagactagacaccgatcaggtgtatggaaacatagcgacatataactttgagaacggccgcgtgcgcgtcgacactgggtcaaactggtctgaagttctgccgcaaattcaagagacaaccgccagaattatctttaatgggaaggacttgaaccttgtcgaacgtagaattgccgccgtgaaccccagtgatccactcgagacgactaaaccggatatgacactgaaagaggctctgaagattgccttcggattcaacgaacctaatggcaatttgcagtatcaggggaaagacatcacagagtttgatttcaatttcgatcagcagacttcccaaaatatcaaaaatcagttggcagagctgaatgccaccaatatctacacggttctcgataaaatcaaacttaacgccaagatgaacatattgattcgagacaaacgcttccactacgaccgcaacaatatagccgtaggcgctgatgagtctgtcgtcaaggaggctcatagggaagttatcaacagcagtactgaagggctgttacttaatatcgacaaggacattcggaagatcctgtccgggtatatcgtggagatcgaggataccgagggcctgaaggaagtcattaacgaccgctatgatatgctgaacatttccagcttacgacaggacggtaagacatttattgactttaaaaagtataacgacaagctacccctgtacatttccaacccaaattacaaagttaatgtgtatgctgtaaccaaggagaacacaatcatcaatccaagcgagaacggcgataccagcacaaatggaatcaaaaagatccttatatttagtaaaaaaggctacgagatcggttgaggatcc

SEQ ID NO: 6 - protein, 587 aa, Artificial Sequence
  Human TPA/synthetic antigen fusion protein
MDAMKRGLCCVLLLCGAVFVSPSSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSETRTISKNTSTSRTHTSEVHGNAEVHASDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 7 - DNA, 2277 bp, Artificial Sequence
  Synthetic coding region for Human TPA/synthetic antigen fusion protein
  CDS (13)..(2268)
gatatcgccaccatggatgcaatgaagagagggctctgctgtgtgctgctgctgtgtggagcagtcttcgtttcgcccagcgaagtgaagcaagaaaatcgacttctgaacgagagcgaaagttcatcacagggtcttctcggatactacttcagtgacttgaatttccaagcaccaatggtggtgactagtagcaccaccggcgatttgagcattcccagctctgagttggagaacattcccagcgaaaatcagtacttccagtctgctatctggtccggattcattaaggttaaaaagtccgacgaatatacatttgctacctcggcggataaccatgtgacaatgtgggtggacgaccaggaagtgatcaacaaggcttcaaactctaataaaatccggctcgagaaggggaggctctaccagatcaaaattcagtaccagcgggaaaaccctacagaaaaaggactcgatttcaagctgtactggacagatagccaaaacaagaaagaagttatcagctcagacaatctgcagttacccgagctcaagcagaagagttctaatacaagcgctgggccaactgtgcccgacagagacaatgatggaatccctgatagtctagaggttgagggatacacggtagatgtcaagaacaaaaggacttttctctcgccttggatctcaaatatccatgagaagaaggggcttaccaagtacaagtcctcccccgagaagtggtctaccgcttccgatccatatagcgatttcgagaaggtcacaggccggatcgataaaaatgtgtctccagaggctagacaccccctggtagcagcctacccgattgtacacgtggacatggagaacatcattctaagcaaaaacgaggaccagtccacacaaaacactgactccgagacccgcaccatatctaaaaacaccagtacttcaaggacccacacctctgaagtgcacggcaatgcggaagtccatgcatcgtttttcgatattggtggctccgtgtcagccggctttagcaatagcaactcctcgacggttgccattgaccactcactgtcattagcaggtgagaggacttgggctgaaactatgggtctgaataccgccgatacggcccggctcaacgcaaatattcggtacgtcaacacagggactgctcctatatataacgtgctgcctacgacaagtcttgtcctgggcaaaaatcagaccctcgcaaccattaaggcaaaggaaaatcagctgagccagatcctcgcccctaacaactattatccatccaaaaatttagcccccatagccctgaacgcccaggacgacttttcctctacccccataactatgaattacaatcagttcctggagctggaaaagacgaagcagctgagactagacaccgatcaggtgtatggaaacatagcgacatataactttgagaacggccgcgtgcgcgtcgacactgggtcaaactggtctgaagttctgccgcaaattcaagagacaaccgccagaattatctttaatgggaaggacttgaaccttgtcgaacgtagaattgccgccgtgaaccccagtgatccactcgagacgactaaaccggatatgacactgaaagaggctctgaagattgccttcggattcaacgaacctaatggcaatttgcagtatcaggggaaagacatcacagagtttgatttcaatttcgatcagcagacttcccaaaatatcaaaaatcagttggcagagctgaatgccaccaatatctacacggttctcgataaaatcaaacttaacgccaagatgaacatattgattcgagacaaacgcttccactacgaccgcaacaatatagccgtaggcgctgatgagtctgtcgtcaaggaggctcatagggaagttatcaacagcagtactgaagggctgttacttaatatcgacaaggacattcggaagatcctgtccgggtatatcgtggagatcgaggataccgagggcctgaaggaagtcattaacgaccgctatgatatgctgaacatttccagcttacgacaggacggtaagacatttattgactttaaaaagtataacgacaagctacccctgtacatttccaacccaaattacaaagttaatgtgtatgctgtaaccaaggagaacacaatcatcaatccaagcgagaacggcgataccagcacaaatggaatcaaaaagatccttatatttagtaaaaaaggctacgagatcggttgaggatcc

SEQ ID NO: 8 - protein, 752 aa, Artificial Sequence
  Human TPA/synthetic antigen fusion protein
MDAMKRGLCCVLLLCGAVFVSPSEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSETRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 9 - DNA, 2418 bp, Artificial Sequence
  Synthetic coding region for Human TPA/synthetic antigen fusion protein
  CDS (13)..(2409)
gatatcgccaccatggatgcaatgaagagagggctctgctgtgtgctgctgctgtgtggagcagtcttcgtttcgcccagcgccggcgggcatggggacgttggcatgcatgtgaaagaaaaggagaaaaacaaggacgaaaacaagcgtaaagacgaagaacgtaataaaacacaggaggaacacttaaaggagatcatgaagcacatagtaaagattgaggtaaaaggcgaagaggctgtaaagaaggaggcagcagaaaaactgttggagaaggtgccttctgacgtcttagagatgtataaggccatcggcggtaagatctatatcgtggacggagacatcactaaacacatatctctcgaagctctctccgaggacaagaaaaagattaaagacatctacgggaaggatgccttattgcacgagcactacgtttacgcaaaggagggctatgagcccgtgctcgttattcagagtagtgaggactacgtcgagaataccgagaaagctctgaatgtgtattacgagatcggaaagattctgtcccgggacatcctgtccaaaatcaaccagccataccagaaattccttgatgttcttaacacaatcaaaaacgcgtcagatagcgacgggcaggatcttctgtttacaaatcaactcaaggaacaccccactgatttcagcgtggagttcctcgagcagaattctaacgaagtccaggaggtgttcgccaaggcatttgcgtactatatcgaaccccagcatcgcgatgtgctccagctgtacgccccggaggcatttaactacatggacaaattcaatgaacaggagattaatctgtctctggaggaactgaaagaccagaggatgctctcccggtatgaaaagtgggaaaagatcaaacagcattaccagcattggtccgactccctgtcagaagaggggcgcggcctgttgaaaaagttgcagattcccatcgagcctaagaaagatgatataatacactctctaagccaggaggagaaggaactcctgaagcggatacaaatcgactcatccgatttccttagcacagaagagaaggagtttctaaaaaaacttcagatagatattagagattcactgagcgaggaagagaaggagctgctcaaccgaattcaagtcgatagttcgaaccccttgtcagaaaaagagaaggaattcctgaaaaagttgaagctcgacatccagccgtacgatattaatcagcggctacaagacaccggcggtctgattgatagccccagcatcaaccttgacgtacggaagcaatataagcgcgacattcaaaatatcgacgccctattacatcaatccataggctccacgctatacaataaaatctatctatacgaaaacatgaatattaacaatctcaccgctacactgggagcggacctggtcgatagtacagacaacacaaagataaacagaggtattttcaacgaattcaaaaagaactttaagtattcgatcagcagtaactatatgattgttgacatcaatgaacggcccgcattagacaatgagaggttgaagtggagaattcaactgagtcctgatactagggccggctatctggagaacgggaaactgatcttacagcgaaacatcgggctggagatcaaggatgtgcagattatcaagcagagcgaaaaagaatacattcgcatcgacgccaaggtggtgcctaagtcaaagatcgataccaagatccaggaagctcagctcaacattaaccaggagtggaataaagctcttggtctgccaaaatacaccaaacttatcacctttaatgtgcacaacaggtatgcctctaatatcgtcgagtcagcatacctgattctcaatgaatggaagaacaatattcagtctgacctgatcaagaaggtcacgaattatctggtggacggaaatggcagattcgtgttcaccgacataactttgccaaacattgccgagcaatacactcatcaggatgaaatttacgagcaagtccactccaaaggtctgtatgttccagagtcaagatcgattctgctccatggtccatccaaaggggttgagcttcgaaacgattctgagggatttatcgctgactttggagccgctgtggatgactacgccggatacctgttggataagaatcagtctgatctcgtgacaaatagcaaaaaattcatagatattttcaaggaggaagggagtaacctgacttcctatggccgcacgaacgaggctgaattttttgcggaagcctttagacttatgcacagcaccgaccatgctgaaaggttgaaggtgcaaaagaatgcccctaaaaccttccagttcataaatgaccagatcaagttcatcatcaactcttgaggatcc

SEQ ID NO: 10 - protein, 799 aa, Artificial Sequence
  Human TPA/synthetic antigen fusion protein
MDAMKRGLCCVLLLCGAVFVSPSAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRMLSRYEKWEKIKQHYQHWSDSLSEEGRGLLKKLQIPIEPKKDDIIHSLSQEEKELLKRIQIDSSDFLSTEEKEFLKKLQIDIRDSLSEEEKELLNRIQVDSSNPLSEKEKEFLKKLKLDIQPYDINQRLQDTGGLIDSPSINLDVRKQYKRDIQNIDALLHQSIGSTLYNKIYLYENMNINNLTATLGADLVDSTDNTKINRGIFNEFKKNFKYSISSNYMIVDINERPALDNERLKWRIQLSPDTRAGYLENGKLILQRNIGLEIKDVQIIKQSEKEYIRIDAKVVPKSKIDTKIQEAQLNINQEWNKALGLPKYTKLITFNVHNRYASNIVESAYLILNEWKNNIQSDLIKKVTNYLVDGNGRFVFTDITLPNIAEQYTHQDEIYEQVHSKGLYVPESRSILLHGPSKGVELRNDSEGFIADFGAAVDDYAGYLLDKNQSDLVTNSKKFIDIFKEEGSNLTSYGRTNEAEFFAEAFRLMHSTDHAERLKVQKNAPKTFQFINDQIKFIINS

SEQ ID NO: 11 - DNA, 3631 bp, Bacillus anthracis
  CDS (685)..(3111)
gcatcatatggcaattatattcatagcgcgttttttcccgttataaaagataaaaactcgaaaacaggaaaataatataccttttattctatacagcaacctaaatattataatcaacttttccatagaaattaatccttttgttatacatctttattcccttaacattgtcaaatttcagttattcattctggatagtcaataaatagattacggttatgttagtatttttttaaaataatagtattaaatagtggaatgcaaatgataaatgggctttaaacaaaactaatgaaataatctacaaatggaatttctccagttttagattaaaccataccaaaaaaatcacactgtcaagaaaaatgatagaatccctacactaattaacataaccaaattggtagttataggtagaaacttatttatttctataataccatgcaaaaaacaaactaaatattctgttccatactattttagtaaattatttagcaagtaaattttggtgtataaacaaagtttatcttaatataaaaaattactttacttttatacagattaaaatgaaaaattttttatgacaagaaatattgcctttaatttatgaggaaataagtaaaattttctacatactttattttattgttgaaatgttcacttataaaaaaggagagattaaatatgaatataaaaaaagaatttataaaagtaattagtatgtcatgtttagtaacagcaattactttgagtggtcccgtctttatcccccttgtacagggggcgggcggtcatggtgatgtaggtatgcacgtaaaagagaaagagaaaaataaagatgagaataagagaaaagatgaagaacgaaataaaacacaggaagagcatttaaaggaaatcatgaaacacattgtaaaaatagaagtaaaaggggaggaagctgttaaaaaagaggcagcagaaaagctacttgagaaagtaccatctgatgttttagagatgtataaagcaattggaggaaagatatatattgtggatggtgatattacaaaacatatatctttagaagcattatctgaagataagaaaaaaataaaagacatttatgggaaagatgctttattacatgaacattatgtatatgcaaaagaaggatatgaacccgtacttgtaatccaatcttcggaagattatgtagaaaatactgaaaaggcactgaacgtttattatgaaataggtaagatattatcaagggatattttaagtaaaattaatcaaccatatcagaaatttttagatgtattaaataccattaaaaatgcatctgattcagatggacaagatcttttatttactaatcagcttaaggaacatcccacagacttttctgtagaattcttggaacaaaatagcaatgaggtacaagaagtatttgcgaaagcttttgcatattatatcgagccacagcatcgtgatgttttacagctttatgcaccggaagcttttaattacatggataaatttaacgaacaagaaataaatctatccttggaagaacttaaagatcaacggatgctgtcaagatatgaaaaatgggaaaagataaaacagcactatcaacactggagcgattctttatctgaagaaggaagaggacttttaaaaaagctgcagattcctattgagccaaagaaagatgacataattcattctttatctcaagaagaaaaagagcttctaaaaagaatacaaattgatagtagtgattttttatctactgaggaaaaagagtttttaaaaaagctacaaattgatattcgtgattctttatctgaagaagaaaaagagcttttaaatagaatacaggtggatagtagtaatcctttatctgaaaaagaaaaagagtttttaaaaaagctgaaacttgatattcaaccatatgatattaatcaaaggttgcaagatacaggagggttaattgatagtccgtcaattaatcttgatgtaagaaagcagtataaaagggatattcaaaatattgatgctttattacatcaatccattggaagtaccttgtacaataaaatttatttgtatgaaaatatgaatatcaataaccttacagcaaccctaggtgcggatttagttgattccactgataatactaaaattaatagaggtattttcaatgaattcaaaaaaaatttcaaatatagtatttctagtaactatatgattgttgatataaatgaaaggcctgcattagataatgagcgtttgaaatggagaatccaattatcaccagatactcgagcaggatatttagaaaatggaaagcttatattacaaagaaacatcggtctggaaataaaggatgtacaaataattaagcaatccgaaaaagaatatataaggattgatgcgaaagtagtgccaaagagtaaaatagatacaaaaattcaagaagcacagttaaatataaatcaggaatggaataaagcattagggttaccaaaatatacaaagcttattacattcaacgtgcataatagatatgcatccaatattgtagaaagtgcttatttaatattgaatgaatggaaaaataatattcaaagtgatcttataaaaaaggtaacaaattacttagttgatggtaatggaagatttgtttttaccgatattactctccctaatatagctgaacaatatacacatcaagatgagatatatgagcaagttcattcaaaagggttatatgttccagaatcccgttctatattactccatggaccttcaaaaggtgtagaattaaggaatgatagtgagggttttatacacgaatttggacatgctgtggatgattatgctggatatctattagataagaaccaatctgatttagttacaaattctaaaaaattcattgatatttttaaggaagaagggagtaatttaacttcgtatgggagaacaaatgaagcggaattttttgcagaagcctttaggttaatgcattctacggaccatgctgaacgtttaaaagttcaaaaaaatgctccgaaaactttccaatttattaacgatcagattaagttcattattaactcataagtaatgtattaaaaattttcaaatggatttaataataataataataataataataacgggaccagccattatgaagcaactaattctagacttgatagtaattcttgggaagcaccagatagtgtaaaaggtggcattgccagaatgatattttatgtgttcgttagatatgaaggcaaaaacaatgatcctgacctagaacttaatgataatgttattaataatttaatgccttttataggaatattagtaaaagtgccgaaaagatcctgttgcaaagcttttaaagaacatattattctatcaagtggctgtatattttgttaattttcaataaattttgtaattaagcatacgtcaaaaaaccgaaatctgagctcagatttcggttttttgacgtatcttatacagatttgcaggttttaatttcatttgctcaatattacaaggatcagtttaaatgcaagtatgatgcacatacatgttgttaaagacaataagacgaaaaatatttctc

SEQ ID NO: 12 - protein, 809 aa, Bacillus anthracis
MNIKKEFIKVISMSCLVTAITLSGPVFIPLVQGAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRMLSRYEKWEKIKQHYQHWSDSLSEEGRGLLKKLQIPIEPKKDDIIHSLSQEEKELLKRIQIDSSDFLSTEEKEFLKKLQIDIRDSLSEEEKELLNRIQVDSSNPLSEKEKEFLKKLKLDIQPYDINQRLQDTGGLIDSPSINLDVRKQYKRDIQNIDALLHQSIGSTLYNKIYLYENMNINNLTATLGADLVDSTDNTKINRGIFNEFKKNFKYSISSNYMIVDINERPALDNERLKWRIQLSPDTRAGYLENGKLILQRNIGLEIKDVQIIKQSEKEYIRIDAKVVPKSKIDTKIQEAQLNINQEWNKALGLPKYTKLITFNVHNRYASNIVESAYLILNEWKNNIQSDLIKKVTNYLVDGNGRFVFTDITLPNIAEQYTHQDEIYEQVHSKGLYVPESRSILLHGPSKGVELRNDSEGFIHEFGHAVDDYAGYLLDKNQSDLVTNSKKFIDIFKEEGSNLTSYGRTNEAEFFAEAFRLMHSTDHAERLKVQKNAPKTFQFINDQIKFIINS

SEQ ID NO: 13 - DNA, 1740 bp, Artificial Sequence
  Synthetic coding region for Human TPA/B. anthracis antigen fusion protein
  CDS (13)..(1731)
gatatcgccaccatggatgcaatgaagagagggctctgctgtgtgctgctgctgtgtggagcagtcttcgtttcgcccagcgccggcgggcatggggacgttggcatgcatgtgaaagaaaaggagaaaaacaaggacgaaaacaagcgtaaagacgaagaacgtaataaaacacaggaggaacacttaaaggagatcatgaagcacatagtaaagattgaggtaaaaggcgaagaggctgtaaagaaggaggcagcagaaaaactgttggagaaggtgccttctgacgtcttagagatgtataaggccatcggcggtaagatctatatcgtggacggagacatcactaaacacatatctctcgaagctctctccgaggacaagaaaaagattaaagacatctacgggaaggatgccttattgcacgagcactacgtttacgcaaaggagggctatgagcccgtgctcgttattcagagtagtgaggactacgtcgagaataccgagaaagctctgaatgtgtattacgagatcggaaagattctgtcccgggacatcctgtccaaaatcaaccagccataccagaaattccttgatgttcttaacacaatcaaaaacgcgtcagatagcgacgggcaggatcttctgtttacaaatcaactcaaggaacaccccactgatttcagcgtggagttcctcgagcagaattctaacgaagtccaggaggtgttcgccaaggcatttgcgtactatatcgaaccccagcatcgcgatgtgctccagctgtacgccccggaggcatttaactacatggacaaattcaatgaacaggagattaatctgtctctggaggaactgaaagaccagaggatgctctcccggtatgaaaagtgggaaaagatcaaacagcattaccagcattggtccgactccctgtcagaagaggggcgcggcctgttgaaaaagttgcagattcccatcgagcctaagaaagatgatataatacactctctaagccaggaggagaaggaactcctgaagcggatacaaatcgactcatccgatttccttagcacagaagagaaggagtttctaaaaaaacttcagatagatattagagattcactgagcgaggaagagaaggagctgctcaaccgaattcaagtcgatagttcgaaccccttgtcagaaaaagagaaggaattcctgaaaaagttgaagctcgacatccagccgtacgatattaatcagcggctacaagacaccggcggtctgattgatagccccagcatcaaccttgacgtacggaagcaatataagcgcgacattcaaaatatcgacgccctattacatcaatccataggctccacgctatacaataaaatctatctatacgaaaacatgaatattaacaatctcaccgctacactgggagcggacctggtcgatagtacagacaacacaaagataaacagaggtattttcaacgaattcaaaaagaactttaagtattcgatcagcagtaactatatgattgttgacatcaatgaacggcccgcattagacaatgagaggttgaagtggagaattcaactgagtcctgatactagggccggctatctggagaacgggaaactgatcttacagcgaaacatcgggctggagatcaaggatgtgcagattatcaagcagagcgaaaaagaatacattcgcatcgacgccaaggtggtgtagggatcc

SEQ ID NO: 14 - protein, 573 aa, Artificial Sequence
  Human TPA/B. anthracis antigen fusion protein
MDAMKRGLCCVLLLCGAVFVSPSAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRMLSRYEKWEKIKQHYQHWSDSLSEEGRGLLKKLQIPIEPKKDDIIHSLSQEEKELLKRIQIDSSDFLSTEEKEFLKKLQIDIRDSLSEEEKELLNRIQVDSSNPLSEKEKEFLKKLKLDIQPYDINQRLQDTGGLIDSPSINLDVRKQYKRDIQNIDALLHQSIGSTLYNKIYLYENMNINNLTATLGADLVDSTDNTKINRGIFNEFKKNFKYSISSNYMIVDINERPALDNERLKWRIQLSPDTRAGYLENGKLILQRNIGLEIKDVQIIKQSEKEYIRIDAKVV

SEQ ID NO: 15 - DNA, 753 bp, Artificial Sequence
  Synthetic coding region for Human TPA/B. anthracis antigen fusion protein
  CDS (13)..(744)
gatatcgccaccatggatgcaatgaagagagggctctgctgtgtgctgctgctgtgtggagcagtcttcgtttcgcccagcgccggcgggcatggggacgttggcatgcatgtgaaagaaaaggagaaaaacaaggacgaaaacaagcgtaaagacgaagaacgtaataaaacacaggaggaacacttaaaggagatcatgaagcacatagtaaagattgaggtaaaaggcgaagaggctgtaaagaaggaggcagcagaaaaactgttggagaaggtgccttctgacgtcttagagatgtataaggccatcggcggtaagatctatatcgtggacggagacatcactaaacacatatctctcgaagctctctccgaggacaagaaaaagattaaagacatctacgggaaggatgccttattgcacgagcactacgtttacgcaaaggagggctatgagcccgtgctcgttattcagagtagtgaggactacgtcgagaataccgagaaagctctgaatgtgtattacgagatcggaaagattctgtcccgggacatcctgtccaaaatcaaccagccataccagaaattccttgatgttcttaacacaatcaaaaacgcgtcagatagcgacgggcaggatcttctgtttacaaatcaactcaaggaacaccccactgatttcagcgtggagttcctcgagcagaattctaacgaagtccaggaggtgttcgccaaggcattttgaggatcc

SEQ ID NO: 16 - protein, 244 aa, Artificial Sequence
  Human TPA/B. anthracis antigen fusion protein
MDAMKRGLCCVLLLCGAVFVSPSAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAF

SEQ ID NO: 17 - DNA, 1788 bp, Artificial Sequence
  Synthetic coding region for Human TPA/synthetic antigen fusion protein
  CDS (13)..(1779)
gatatcgccaccatggatgcaatgaagagagggctctgctgtgtgctgctgctgtgtggagcagtcttcgtttcgcccagcagcgctgggccaactgtgcccgacagagacaatgatggaatccctgatagtctagaggttgagggatacacggtagatgtcaagaacaaaaggacttttctctcgccttggatctcaaatatccatgagaagaaggggcttaccaagtacaagtcctcccccgagaagtggtctaccgcttccgatccatatagcgatttcgagaaggtcacaggccggatcgataaacaggtgtctccagaggctagacaccccctggtagcagcctacccgattgtacacgtggacatggagaacatcattctaagcaaaaacgaggaccagtccacacaaaacactgactccgagacccgcaccatatctaaacagaccagtacttcaaggacccacacctctgaagtgcacggcaatgcggaagtccatgcatcgtttttcgatattggtggctccgtgtcagccggctttagcaatagccagtcctcgacggttgccattgaccactcactgtcattagcaggtgagaggacttgggctgaaactatgggtctgaataccgccgatacggcccggctcaacgcaaatattcggtacgtcaacacagggactgctcctatatataacgtgctgcctacgacaagtcttgtcctgggcaaacagcagaccctcgcaaccattaaggcaaaggaaaatcagctgagccagatcctcgcccctaacaactattatccatccaaaaatttagcccccatagccctgaacgcccaggacgacttttcctctacccccataactatgaattacaatcagttcctggagctggaaaagacgaagcagctgagactagacaccgatcaggtgtatggaaacatagcgacatataactttgagaacggccgcgtgcgcgtcgacactgggtcacagtggtctgaagttctgccgcaaattcaagagacaaccgccagaattatctttaatgggaaggacttgaaccttgtcgaacgtagaattgccgccgtgcagcccagtgatccactcgagacgactaaaccggatatgacactgaaagaggctctgaagattgccttcggattcaacgaacctaatggcaatttgcagtatcaggggaaagacatcacagagtttgatttcaatttcgatcagcagacttcccaaaatatcaaaaatcagttggcagagctgcaggccaccaatatctacacggttctcgataaaatcaaacttaacgccaagatgaacatattgattcgagacaaacgcttccactacgaccgcaacaatatagccgtaggcgctgatgagtctgtcgtcaaggaggctcatagggaagttatccagagcagtactgaagggctgttacttaatatcgacaaggacattcggaagatcctgtccgggtatatcgtggagatcgaggataccgagggcctgaaggaagtcattaacgaccgctatgatatgctgcagatttccagcttacgacaggacggtaagacatttattgactttaaaaagtataacgacaagctacccctgtacatttccaacccaaattacaaagttaatgtgtatgctgtaaccaaggagaacacaatcatccagccaagcgagaacggcgataccagcacaaatggaatcaaaaagatccttatatttagtaaaaaaggctacgagatcggttgaggatcc

SEQ ID NO: 18 - protein, 589 aa, Artificial Sequence
  Human TPA/synthetic antigen fusion protein
MDAMKRGLCCVLLLCGAVFVSPSSAGPTVPDRDNDGIPDSLEVEGYTVDVKNKRTFLSPWISNIHEKKGLTKYKSSPEKWSTASDPYSDFEKVTGRIDKQVSPEARHPLVAAYPIVHVDMENIILSKNEDQSTQNTDSETRTISKQTSTSRTHTSEVHGNAEVHASFFDIGGSVSAGFSNSQSSTVAIDHSLSLAGERTWAETMGLNTADTARLNANIRYVNTGTAPIYNVLPTTSLVLGKQQTLATIKAKENQLSQILAPNNYYPSKNLAPIALNAQDDFSSTPITMNYNQFLELEKTKQLRLDTDQVYGNIATYNFENGRVRVDTGSQWSEVLPQIQETTARIIFNGKDLNLVERRIAAVQPSDPLETTKPDMTLKEALKIAFGFNEPNGNLQYQGKDITEFDFNFDQQTSQNIKNQLAELQATNIYTVLDKIKLNAKMNILIRDKRFHYDRNNIAVGADESVVKEAHREVIQSSTEGLLLNIDKDIRKILSGYIVEIEDTEGLKEVINDRYDMLQISSLRQDGKTFIDFKKYNDKLPLYISNPNYKVNVYAVTKENTIIQPSENGDTSTNGIKKILIFSKKGYEIG

SEQ ID NO: 19 - DNA, 2418 bp, Artificial Sequence
  Synthetic coding region for Human TPA/synthetic antigen fusion protein
  CDS (13)..(2409)
gatatcgccaccatggatgcaatgaagagagggctctgctgtgtgctgctgctgtgtggagcagtcttcgtttcgcccagcgccggcgggcatggggacgttggcatgcatgtgaaagaaaaggagaaaaacaaggacgaaaacaagcgtaaagacgaagaacgtcagaaaacacaggaggaacacttaaaggagatcatgaagcacatagtaaagattgaggtaaaaggcgaagaggctgtaaagaaggaggcagcagaaaaactgttggagaaggtgccttctgacgtcttagagatgtataaggccatcggcggtaagatctatatcgtggacggagacatcactaaacacatatctctcgaagctctctccgaggacaagaaaaagattaaagacatctacgggaaggatgccttattgcacgagcactacgtttacgcaaaggagggctatgagcccgtgctcgttattcagagtagtgaggactacgtcgagaataccgagaaagctctgaatgtgtattacgagatcggaaagattctgtcccgggacatcctgtccaaaatcaaccagccataccagaaattccttgatgttcttaacacaatcaaacaggcgtcagatagcgacgggcaggatcttctgtttacaaatcaactcaaggaacaccccactgatttcagcgtggagttcctcgagcagaattctaacgaagtccaggaggtgttcgccaaggcatttgcgtactatatcgaaccccagcatcgcgatgtgctccagctgtacgccccggaggcatttaactacatggacaaattcaatgaacaggagattcagctgtctctggaggaactgaaagaccagaggatgctctcccggtatgaaaagtgggaaaagatcaaacagcattaccagcattggtccgactccctgtcagaagaggggcgcggcctgttgaaaaagttgcagattcccatcgagcctaagaaagatgatataatacactctctaagccaggaggagaaggaactcctgaagcggatacaaatcgactcatccgatttccttagcacagaagagaaggagtttctaaaaaaacttcagatagatattagagattcactgagcgaggaagagaaggagctgctcaaccgaattcaagtcgatagttcgaaccccttgtcagaaaaagagaaggaattcctgaaaaagttgaagctcgacatccagccgtacgatattaatcagcggctacaagacaccggcggtctgattgatagccccagcatcaaccttgacgtacggaagcaatataagcgcgacattcaaaatatcgacgccctattacatcaatccataggctccacgctatacaataaaatctatctatacgaaaacatgaatattaaccagctcaccgctacactgggagcggacctggtcgatagtacagacaacacaaagataaacagaggtattttcaacgaattcaaaaagaactttaagtattcgatcagcagtaactatatgattgttgacatcaatgaacggcccgcattagacaatgagaggttgaagtggagaattcaactgagtcctgatactagggccggctatctggagaacgggaaactgatcttacagcgaaacatcgggctggagatcaaggatgtgcagattatcaagcagagcgaaaaagaatacattcgcatcgacgccaaggtggtgcctaagtcaaagatcgataccaagatccaggaagctcagctcaacattaaccaggagtggaataaagctcttggtctgccaaaatacaccaaacttatcacctttaatgtgcacaacaggtatgcctctaatatcgtcgagtcagcatacctgattctcaatgaatggaagaacaatattcagtctgacctgatcaagaaggtcacgaattatctggtggacggaaatggcagattcgtgttcaccgacataactttgccaaacattgccgagcaatacactcatcaggatgaaatttacgagcaagtccactccaaaggtctgtatgttccagagtcaagatcgattctgctccatggtccatccaaaggggttgagcttcgacaggattctgagggatttatcgctgactttggagccgctgtggatgactacgccggatacctgttggataagcagcagtctgatctcgtgacaaatagcaaaaaattcatagatattttcaaggaggaagggagtcagctgacttcctatggccgcacgaacgaggctgaattttttgcggaagcctttagacttatgcacagcaccgaccatgctgaaaggttgaaggtgcaaaagaatgcccctaaaaccttccagttcataaatgaccagatcaagttcatcatcaactcttgaggatcc

SEQ ID NO: 20 - protein, 799 aa, Artificial Sequence
  Human TPA/synthetic antigen fusion protein
MDAMKRGLCCVLLLCGAVFVSPSAGGHGDVGMHVKEKEKNKDENKRKDEERQKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKQASDSDGQDLLFTNQLKEHPTDFSVEFLEQNSNEVQEVFAKAFAYYIEPQHRDVLQLYAPEAFNYMDKFNEQEIQLSLEELKDQRMLSRYEKWEKIKQHYQHWSDSLSEEGRGLLKKLQIPIEPKKDDIIHSLSQEEKELLKRIQIDSSDFLSTEEKEFLKKLQIDIRDSLSEEEKELLNRIQVDSSNPLSEKEKEFLKKLKLDIQPYDINQRLQDTGGLIDSPSINLDVRKQYKRDIQNIDALLHQSIGSTLYNKIYLYENMNINQLTATLGADLVDSTDNTKINRGIFNEFKKNFKYSISSNYMIVDINERPALDNERLKWRIQLSPDTRAGYLENGKLILQRNIGLEIKDVQIIKQSEKEYIRIDAKVVPKSKIDTKIQEAQLNINQEWNKALGLPKYTKLITFNVHNRYASNIVESAYLILNEWKNNIQSDLIKKVTNYLVDGNGRFVFTDITLPNIAEQYTHQDEIYEQVHSKGLYVPESRSILLHGPSKGVELRQDSEGFIADFGAAVDDYAGYLLDKQQSDLVTNSKKFIDIFKEEGSQLTSYGRTNEAEFFAEAFRLMHSTDHAERLKVQKNAPKTFQFINDQIKFIINS

SEQ ID NO: 21 - DNA, 2292 bp, Artificial Sequence
  Synthetic coding region
atgaagaagaggaaggtgctgatccccctgatggccctgagcaccatcctggtgagcagcaccggcaacctggaggtgatccaggccgaggtgaagcaggagaacaggctgctgaacgagagcgagagcagcagccagggcctgctgggctactacttcagcgacctgaacttccaggcccccatggtggtgaccagcagcaccaccggcgacctgagcatccccagcagcgagctggagaacatccccagcgagaaccagtacttccagagcgccatctggagcggcttcatcaaggtgaagaagagcgacgagtacaccttcgccaccagcgccgacaaccacgtgaccatgtgggtggacgaccaggaggtgatcaacaaggccagcaacagcaacaagatcaggctggagaagggcaggctgtaccagatcaagatccagtaccagagggagaaccccaccgagaagggcctggacttcaagctgtactggaccgacagccagaacaagaaggaggtgatcagcagcgacaacctgcagctgcccgagctgaagcagaagagcagcaacagcaggaagaagaggagcaccagcgccggccccaccgtgcccgacagggacaacgacggcatccccgacagcctggaggtggagggctacaccgtggacgtgaagaacaagaggaccttcctgagcccctggatcagcaacatccacgagaagaagggcctgaccaagtacaagagcagccccgagaagtggagcaccgccagcgacccctacagcgacttcgagaaggtgaccggcaggatcgacaagaacgtgagccccgaggccaggcaccccctggtggccgcctaccccatcgtgcacgtggacatggagaacatcatcctgagcaagaacgaggaccagagcacccagaacaccgacagcgagaccaggaccatcagcaagaacaccagcaccagcaggacccacaccagcgaggtgcacggcaacgccgaggtgcacgccagcttcttcgacatcggcggcagcgtgagcgccggcttcagcaacagcaacagcagcaccgtggccatcgaccacagcctgagcctggccggcgagaggacctgggccgagaccatgggcctgaacaccgccgacaccgccaggctgaacgccaacatcaggtacgtgaacaccggcaccgcccccatctacaacgtgctgcccaccaccagcctggtgctgggcaagaaccagaccctggccaccatcaaggccaaggagaaccagctgagccagatcctggcccccaacaactactaccccagcaagaacctggcccccatcgccctgaacgcccaggacgacttcagcagcacccccatcaccatgaactacaaccagttcctggagctggagaagaccaagcagctgaggctggacaccgaccaggtgtacggcaacatcgccacctacaacttcgagaacggcagggtgagggtggacaccggcagcaactggagcgaggtgctgccccagatccaggagaccaccgccaggatcatcttcaacggcaaggacctgaacctggtggagaggaggatcgccgccgtgaaccccagcgaccccctggagaccaccaagcccgacatgaccctgaaggaggccctgaagatcgccttcggcttcaacgagcccaacggcaacctgcagtaccagggcaaggacatcaccgagttcgacttcaacttcgaccagcagaccagccagaacatcaagaaccagctggccgagctgaacgccaccaacatctacaccgtgctggacaagatcaagctgaacgccaagatgaacatcctgatcagggacaagaggttccactacgacaggaacaacatcgccgtgggcgccgacgagagcgtggtgaaggaggcccacagggaggtgatcaacagcagcaccgagggcctgctgctgaacatcgacaaggacatcaggaagatcctgagcggctacatcgtggagatcgaggacaccgagggcctgaaggaggtgatcaacgacaggtacgacatgctgaacatcagcagcctgaggcaggacggcaagaccttcatcgacttcaagaagtacaacgacaagctgcccctgtacatcagcaaccccaactacaaggtgaacgtgtacgccgtgaccaaggagaacaccatcatcaaccccagcgagaacggcgacaccagcaccaacggcatcaagaagatcctgatcttcagcaagaagggctacgagatcggc

SEQ ID NO: 22 - DNA, 2427 bp, Artificial Sequence
  Synthetic coding region
atgaacatcaagaaggagttcatcaaggtgatcagcatgagctgcctggtgaccgccatcaccctgagcggccccgtgttcatccccctggtgcagggcgccggcggccacggcgacgtgggcatgcacgtgaaggagaaggagaagaacaaggacgagaacaagaggaaggacgaggagaggaacaagacccaggaggagcacctgaaggagatcatgaagcacatcgtgaagatcgaggtgaagggcgaggaggccgtgaagaaggaggccgccgagaagctgctggagaaggtgcccagcgacgtgctggagatgtacaaggccatcggcggcaagatctacatcgtggacggcgacatcaccaagcacatcagcctggaggccctgagcgaggacaagaagaagatcaaggacatctacggcaaggacgccctgctgcacgagcactacgtgtacgccaaggagggctacgagcccgtgctggtgatccagagcagcgaggactacgtggagaacaccgagaaggccctgaacgtgtactacgagatcggcaagatcctgagcagggacatcctgagcaagatcaaccagccctaccagaagttcctggacgtgctgaacaccatcaagaacgccagcgacagcgacggccaggacctgctgttcaccaaccagctgaaggagcaccccaccgacttcagcgtggagttcctggagcagaacagcaacgaggtgcaggaggtgttcgccaaggccttcgcctactacatcgagccccagcacagggacgtgctgcagctgtacgcccccgaggccttcaactacatggacaagttcaacgagcaggagatcaacctgagcctggaggagctgaaggaccagaggatgctgagcaggtacgagaagtgggagaagatcaagcagcactaccagcactggagcgacagcctgagcgaggagggcaggggcctgctgaagaagctgcagatccccatcgagcccaagaaggacgacatcatccacagcctgagccaggaggagaaggagctgctgaagaggatccagatcgacagcagcgacttcctgagcaccgaggagaaggagttcctgaagaagctgcagatcgacatcagggacagcctgagcgaggaggagaaggagctgctgaacaggatccaggtggacagcagcaaccccctgagcgagaaggagaaggagttcctgaagaagctgaagctggacatccagccctacgacatcaaccagaggctgcaggacaccggcggcctgatcgacagccccagcatcaacctggacgtgaggaagcagtacaagagggacatccagaacatcgacgccctgctgcaccagagcatcggcagcaccctgtacaacaagatctacctgtacgagaacatgaacatcaacaacctgaccgccaccctgggcgccgacctggtggacagcaccgacaacaccaagatcaacaggggcatcttcaacgagttcaagaagaacttcaagtacagcatcagcagcaactacatgatcgtggacatcaacgagaggcccgccctggacaacgagaggctgaagtggaggatccagctgagccccgacaccagggccggctacctggagaacggcaagctgatcctgcagaggaacatcggcctggagatcaaggacgtgcagatcatcaagcagagcgagaaggagtacatcaggatcgacgccaaggtggtgcccaagagcaagatcgacaccaagatccaggaggcccagctgaacatcaaccaggagtggaacaaggccctgggcctgcccaagtacaccaagctgatcaccttcaacgtgcacaacaggtacgccagcaacatcgtggagagcgcctacctgatcctgaacgagtggaagaacaacatccagagcgacctgatcaagaaggtgaccaactacctggtggacggcaacggcaggttcgtgttcaccgacatcaccctgcccaacatcgccgagcagtacacccaccaggacgagatctacgagcaggtgcacagcaagggcctgtacgtgcccgagagcaggagcatcctgctgcacggccccagcaagggcgtggagctgaggaacgacagcgagggcttcatccacgagttcggccacgccgtggacgactacgccggctacctgctggacaagaaccagagcgacctggtgaccaacagcaagaagttcatcgacatcttcaaggaggagggcagcaacctgaccagctacggcaggaccaacgaggccgagttcttcgccgaggccttcaggctgatgcacagcaccgaccacgccgagaggctgaaggtgcagaagaacgcccccaagaccttccagttcatcaacgaccagatcaagttcatcatcaacagc

SEQ ID NO: 23 - DNA, 2295 bp, Artificial Sequence
  Synthetic coding region
atgaaaaagcgtaaggtgctgatccctctcatggcgctgtcaactatcttggtgagtagcaccggcaacctggaagtgatccaagccgaagtgaagcaagaaaatcgacttctgaacgagagcgaaagttcatcacagggtcttctcggatactacttcagtgacttgaatttccaagcaccaatggtggtgactagtagcaccaccggcgatttgagcattcccagctctgagttggagaacattcccagcgaaaatcagtacttccagtctgctatctggtccggattcattaaggttaaaaagtccgacgaatatacatttgctacctcggcggataaccatgtgacaatgtgggtggacgaccaggaagtgatcaacaaggcttcaaactctaataaaatccggctcgagaaggggaggctctaccagatcaaaattcagtaccagcgggaaaaccctacagaaaaaggactcgatttcaagctgtactggacagatagccaaaacaagaaagaagttatcagctcagacaatctgcagttacccgagctcaagcagaagagttctaattctaggaagaaaagatctacatccgcagggccaactgtgcccgacagagacaatgatggaatccctgatagtctagaggttgagggatacacggtagatgtcaagaacaaaaggacttttctctcgccttggatatcaaatatccatgagaagaaggggcttaccaagtacaagtcctcccccgagaagtggtctaccgcttccgatccatatagcgatttcgagaaggtcacaggccggatcgataaaaatgtgtctccagaggctagacaccccctggtagcagcctacccgattgtacacgtggacatggagaacatcattctaagcaaaaacgaggaccagtccacacaaaacactgactccgagacccgcaccatatctaaaaacaccagtacttcaaggacccacacctctgaagtgcacggcaatgcggaagtccatgcatcgtttttcgatattggtggatccgtgtcagccggctttagcaatagcaactcctcgacggttgccattgaccactcactgtcattagcaggtgagaggacttgggctgaaactatgggtctgaataccgccgatacggcccggctcaacgcaaatattcggtacgtcaacacagggactgctcctatatataacgtgctgcctacgacaagtcttgtcctgggcaaaaatcagaccctcgcaaccattaaggcaaaggaaaatcagctgagccagatcctcgcccctaacaactattatccatccaaaaatttagcccccatagccctgaacgcccaggacgacttttcctctacccccataactatgaattacaatcagttcctggagctggaaaagacgaagcagctgagactagacaccgatcaggtgtatggaaacatagcgacatataactttgagaacggccgcgtgcgcgtcgacactgggtcaaactggtctgaagttctgccgcaaattcaagagacaaccgccagaattatctttaatgggaaggacttgaaccttgtcgaacgtagaattgccgccgtgaaccccagtgatccactcgagacgactaaaccggatatgacactgaaagaggctctgaagattgccttcggattcaacgaacctaatggcaatttgcagtatcaggggaaagacatcacagagtttgatttcaatttcgatcagcagacttcccaaaatatcaaaaatcagttggcagagctgaatgccaccaatatctacacggttctcgataaaatcaaacttaacgccaagatgaacatattgattcgagacaaacgcttccactacgaccgcaacaatatagccgtaggcgctgatgagtctgtcgtcaaggaggctcatagggaagttatcaacagcagtactgaagggctgttacttaatatcgacaaggacattcggaagatcctgtccgggtatatcgtggagatcgaggataccgagggcctgaaggaagtcattaacgaccgctatgatatgctgaacatttccagcttacgacaggacggtaagacatttattgactttaaaaagtataacgacaagctacccctgtacatttccaacccaaattacaaagttaatgtgtatgctgtaaccaaggagaacacaatcatcaatccaagcgagaacggcgataccagcacaaatggaatcaaaaagatccttatatttagtaaaaaaggctacgagatcggttga

SEQ ID NO: 24 - DNA, 2292 bp, Artificial Sequence
  Synthetic coding region
atgaaaaagaggaaggtgctgatccctctcatggccctgtctaccatcctggttagtagcacagggaacctggaagtgattcaggccgaggttaagcaagagaataggctgctcaacgagtcagaatcttcgtcacagggattattgggttactatttttcggacctgaatttccaggccccaatggtcgttacaagctccacaacaggcgacctgtctatccccagctccgaattggagaacatccctagcgagaaccaatactttcaaagcgctatttggtcaggcttcataaaagtgaagaagtctgacgaatacacgtttgcaacatctgccgataaccacgtcactatgtgggtcgatgaccaggaagtcatcaacaaggctagtaatagcaacaaaatcagactggagaaagggagattgtaccagatcaagatccagtaccaacgggaaaacccaacagagaagggcctcgattttaaactgtattggactgactctcagaataagaaggaagtgattagcagcgacaatttacaattacccgagttgaaacagaagagctctaattcaaggaaaaagagatctacctccgccggaccaacagttccagatagggataatgatggaatccctgactcactggaggtcgagggttacaccgtggacgtgaaaaacaaacgcactttcctatcaccctggatctccaacattcacgagaagaagggtctgactaagtacaaatccagcccagagaaatggagcaccgcaagtgatccttatagtgacttcgagaaggtgacgggccggatagacaagaacgtatcacccgaagctcgtcatcctctggtcgccgcctaccctattgtgcatgtggacatggaaaacatcatcctgagtaagaacgaagaccagagcactcagaacaccgactccgagacacgaacgatatctaagaatacatccacctcacgcactcataccagcgaagtgcacggtaacgctgaagtgcacgcgtccttcttcgacatcggcgggtccgtgtccgctggattttccaactccaactcttcgaccgtagctattgaccacagcctgagccttgccggagaaaggacatgggcggagactatgggcctgaatacggctgatacggcacggctcaatgccaacatcagatacgtgaacaccgggacagcccctatttacaatgtgctcccaaccacatcactcgtactgggaaaaaaccagaccctagctactattaaagcgaaagaaaatcagttgtcacagatactggcacccaacaattattatccaagcaaaaacctggcacccatcgcactcaatgcgcaggatgactttagtagtacacccattacaatgaactacaatcagttccttgagctcgagaagaccaagcaactgagactcgacactgaccaggtgtatggtaatatcgccacctacaacttcgaaaacggaagggtgcgggtagatacgggctccaactggtcggaggtgttacctcagatccaggaaaccaccgcccgcattattttcaacggcaaggatctaaatctcgttgagcgccggatagcagctgtcaatcccagtgatccgctcgaaaccaccaagcccgacatgactttgaaggaagctcttaagattgcctttgggtttaacgagcccaatgggaatctgcagtaccagggcaaagatattaccgagtttgattttaactttgatcagcagacaagccagaacattaagaatcaattagccgagctgaatgccactaacatctatactgttttggacaagattaaacttaatgcaaagatgaatatactaatacgagataagcgcttccattatgatcgaaataacatcgcagttggcgccgacgaatcagttgtgaaggaggcccacagagaggtcattaattcctctacagagggtcttcttctgaatatcgacaaggacatacgtaagatcctgagcggatatattgtagaaatcgaagatactgaggggctgaaagaggtcatcaacgaccgctatgatatgcttaacatctctagtttgcggcaagatggaaagactttcattgatttcaagaaatacaacgataaactcccgctgtatatctccaacccaaattataaggtgaatgtgtacgctgtcaccaaagagaataccattattaacccgtctgagaatggcgacacctccacgaatgggataaaaaaaatccttatcttcagtaaaaaaggctacgagatcggg

SEQ ID NO: 25 - DNA, 2292 bp, Artificial Sequence
  Synthetic coding region
atgaaaaaaaggaaggtgttgatcccccttatggccctttccaccatcttggtatcctcaaccggcaacctggaggttattcaagccgaagtgaagcaggaaaatagactgctgaacgaatccgaatctagttctcagggtctgctgggctactattttagcgacctcaatttccaggcaccaatggtcgtgacttcgagcaccacaggcgacttgagcattccctcttccgaactcgagaacataccaagcgagaatcagtattttcagtccgcaatctggtcgggttttatcaaagtaaaaaagagcgacgaatacactttcgctacgtcagccgataatcatgtgaccatgtgggtggatgaccaagaggtcatcaataaggcgagtaactctaacaagattcgactggaaaagggacgcctctatcagattaagattcagtaccagcgtgagaaccccactgaaaagggtctggactttaagctgtattggacggatagtcagaataaaaaggaggtgatcagttcagacaacttgcaattgcctgagctgaaacagaagtccagcaactctcggaagaagcgcagtactagcgctggcccaacagtccccgaccgcgacaatgatgggattcccgattctttggaagtggagggatacacagtggacgtgaagaacaagagaacattcctgagtccatggattagtaatatccatgagaaaaaaggtctaaccaaatacaaaagcagcccagagaagtggtcaacagcatcggatccttactccgatttcgagaaagttactggcaggattgacaagaacgtatctccggaggccaggcatcctctcgtcgccgcttacccgatcgtccacgtcgacatggagaacatcatcctgagtaagaatgaggatcaaagcactcagaatactgattccgagacacggacaatcagtaagaatacctcaacgagcaggacacacacctctgaagttcacggcaatgccgaggtgcacgcttcattcttcgatatcggaggatccgtgagcgcgggcttcagcaactctaactcttccactgtagcgatcgatcatagcctctccctagccggagagcggacatgggctgagaccatggggttgaatactgcagatacagcaagactgaacgccaatattaggtatgtgaatacaggtaccgcccccatctacaacgtccttcctaccacctcactggtgttaggcaaaaatcagaccctcgccaccattaaggcaaaagagaatcaactctcacagatactggccccaaacaactattacccatctaagaatttagctcccattgctttaaacgcccaggacgattttagctcaacgcctatcaccatgaattataaccagttcctggaactggaaaaaactaagcagctccgcctggacaccgatcaggtgtatggcaacatcgccacatacaatttcgaaaatgggcgcgttcgggtggacaccgggtccaactggagtgaagtcctaccccaaatccaggaaaccactgctcgaatcatcttcaatggaaaagacctgaatcttgtggagcggcgaatcgccgctgtgaatccttccgaccctctggaaacgacgaagcccgacatgactttgaaagaggcgctaaaaatcgcttttggatttaatgaaccgaacggcaacttacaatatcaagggaaggacattaccgagttcgactttaactttgatcagcagacctcgcagaacataaagaatcagctcgctgagctgaacgcaacgaatatatacaccgtcctggacaaaattaagcttaacgccaagatgaacatcctcattagagacaagagatttcactacgataggaataacattgccgttggagccgatgagtctgtggtgaaagaggcacaccgcgaggtcattaactccagcactgaagggctgctgctgaacattgacaaggatattagaaaaatcctgagcgggtacatcgttgagatcgaagataccgagggacttaaggaagttataaacgaccgttatgacatgttaaacatatcaagcctccggcaggacggtaagacatttatagatttcaagaaatacaacgataagcttcctctttacatctcaaatcccaactataaggtgaatgtttatgcagtaacaaaagaaaatacaattattaatccatccgagaacggcgatacatctactaacgggataaaaaaaatcctcatcttctccaagaaaggctacgagataggg

SEQ ID NO: 26 - DNA, 2430 bp, Artificial Sequence
  Synthetic coding region
atgaacatcaaaaaagagtttataaaggtgattagcatgagctgcctggtcactgccattaccctgagtggcccagtgtttatccctctcgtccagggcgccggcgggcatggggacgttggcatgcatgtgaaagaaaaggagaaaaacaaggacgaaaacaagcgtaaagacgaagaacgtaataaaacacaggaggaacacttaaaggagatcatgaagcacatagtaaagattgaggtaaaaggcgaagaggctgtaaagaaggaggcagcagaaaaactgttggagaaggtgccttctgacgtcttagagatgtataaggccatcggcggtaagatctatatcgtggacggagacatcactaaacacatatctctcgaagctctctccgaggacaagaaaaagattaaagacatctacgggaaggatgccttattgcacgagcactacgtttacgcaaaggagggctatgagcccgtgctcgttattcagagtagtgaggactacgtcgagaataccgagaaagctctgaatgtgtattacgagatcggaaagattctgtcccgggatatcctgtccaaaatcaaccagccataccagaaattccttgatgttcttaacacaatcaaaaacgcgtcagatagcgacgggcaggatcttctgtttacaaatcaactcaaggaacaccccactgatttcagcgtggagttcctcgagcagaattctaacgaagtccaggaggtgttcgccaaggcatttgcgtactatatcgaaccccagcatcgcgatgtgctccagctgtacgccccggaggcatttaactacatggacaaattcaatgaacaggagattaatctgtctctggaggaactgaaagaccagaggatgctctcccggtatgaaaagtgggaaaagatcaaacagcattaccagcattggtccgactccctgtcagaagaggggcgcggcctgttgaaaaagttgcagattcccatcgagcctaagaaagatgatataatacactctctaagccaggaggagaaggaactcctgaagcggatacaaatcgactcatccgatttccttagcacagaagagaaggagtttctaaaaaaacttcagatagatattagagattcactgagcgaggaagagaaggagctgctcaaccgaattcaagtcgatagttcgaaccccttgtcagaaaaagagaaggaattcctgaaaaagttgaagctcgacatccagccgtacgatattaatcagcggctacaagacaccggcggtctgattgatagccccagcatcaaccttgacgtacggaagcaatataagcgcgacattcaaaatatcgacgccctattacatcaatccataggatccacgctatacaataaaatctatctatacgaaaacatgaatattaacaatctcaccgctacactgggagcggacctggtcgatagtacagacaacacaaagataaacagaggtattttcaacgaattcaaaaagaactttaagtattcgatcagcagtaactatatgattgttgacatcaatgaacggcccgcattagacaatgagaggttgaagtggagaattcaactgagtcctgatactagggccggctatctggagaacgggaaactgatcttacagcgaaacatcgggctggagatcaaggatgtgcagattatcaagcagagcgaaaaagaatacattcgcatcgacgccaaggtggtgcctaagtcaaagatcgataccaagatccaggaagctcagctcaacattaaccaggagtggaataaagctcttggtctgccaaaatacaccaaacttatcacctttaatgtgcacaacaggtatgcctctaatatcgtcgagtcagcatacctgattctcaatgaatggaagaacaatattcagtctgacctgatcaagaaggtcacgaattatctggtggacggaaatggcagattcgtgttcaccgacataactttgccaaacattgccgagcaatacactcatcaggatgaaatttacgagcaagtccactccaaaggtctgtatgttccagagtcaagatcgattctgctccatggtccatccaaaggggttgagcttcgaaacgattctgagggatttatccacgagtttggacacgctgtggatgactacgccggatacctgttggataagaatcagtctgatctcgtgacaaatagcaaaaaattcatagatattttcaaggaggaagggagtaacctgacttcctatggccgcacgaacgaggctgaattttttgcggaagcctttagacttatgcacagcaccgaccatgctgaaaggttgaaggtgcaaaagaatgcccctaaaaccttccagttcataaatgaccagatcaagttcatcatcaactcttga

SEQ ID NO: 27 - DNA, 2427 bp, Artificial Sequence
  Synthetic coding region
atgaatattaaaaaggagtttattaaggttatctctatgtcctgcttggtgacagcgataacactgtcaggaccagtgttcataccccttgtccagggggccggcggtcatggcgatgtaggtatgcatgtgaaagagaaggaaaaaaataaagacgagaacaagaggaaggacgaggaaaggaataagacccaagaggagcacctgaaagagatcatgaagcatattgtgaaaatcgaggtgaagggggaagaggccgtgaaaaaagaagcagctgagaagctgctagagaaagtgccttctgacgtcctcgagatgtacaaagcaatcggcggcaagatttacattgttgatggtgacataacaaagcatatttctctggaggccttaagtgaagataagaaaaaaatcaaagacatttacggaaaggacgccctcctgcacgagcattatgtctacgctaaagaaggctacgaacccgtgctcgtcatccagtcatctgaggattatgtggagaacaccgaaaaagctttgaacgtctattacgaaattgggaaaattctgtctagagacattctcagcaagatcaaccagccataccaaaaattcctagacgttctaaatacgatcaagaatgccagtgactccgacgggcaggatctgttgtttacgaaccagcttaaggagcatcctaccgatttttctgtcgaattccttgagcagaactccaatgaagttcaagaggtctttgctaaggctttcgcgtactacatcgagcctcagcaccgggacgtgctgcagctctacgcccctgaagctttcaattatatggacaagttcaatgaacaggaaattaacctgagtttagaagaactgaaagaccaaagaatgttgtccagatacgagaagtgggagaagatcaagcagcactatcagcactggtccgattcccttagcgaagaagggcgcgggctgcttaaaaagctgcagattccgatcgagccgaagaaagacgatataattcattcactgagccaggaggaaaaggagctcctcaaacggatccagatcgactcgtccgatttcctatccacagaggaaaaggaatttcttaaaaaactccagattgatatacgggactcattatctgaggaggaaaaggaactcctgaaccggatccaggtcgatagtagcaaccccctgtcagaaaaggaaaaggagtttctcaaaaaacttaagctggatatccaaccatacgacatcaaccagcgactgcaggatactggaggcttgatagattctccctccataaacctggacgtgaggaagcagtataagagggatatccagaatatcgatgccctgctgcaccaatctatcggaagtactctttacaacaaaatctatctgtatgagaacatgaatattaataacctgactgctaccttgggcgccgacctggtggactcgacggacaacaccaaaatcaaccgggggatcttcaatgaatttaagaaaaatttcaagtactccatttccagtaattatatgatagttgatatcaacgagcggccagcactggacaacgagagattaaagtggcgaattcaactgagtcccgatacacgcgccggttacctcgagaacggtaagttgatcttgcagcgaaacatcggactcgagattaaggatgtacagatcatcaagcagagcgagaaggagtacattcgtatcgacgctaaagtggtaccaaaaagcaaaattgacaccaagatacaggaggcacagctgaacataaatcaagaatggaataaagccctcggtctgcctaagtatactaagctaatcacctttaacgtgcacaatagatatgccagcaatattgtcgagagcgcatacctaattctgaacgaatggaaaaacaatatccaaagcgacctgataaaaaaggtgactaattatctcgttgatggcaatggccgcttcgttttcaccgatattactctccccaacatcgcagaacagtacactcatcaggacgagatttatgaacaggtgcacagtaaggggctgtatgtccctgagagccgctctatccttcttcacggaccctcaaagggcgtagagttaaggaatgacagcgaggggttcattcacgagtttggccacgcagtggatgattacgctggatatctcctggataagaaccagtccgacctggtgacaaactcaaagaagttcatcgatatctttaaggaggaaggcagcaatttgaccagctacggacgcacaaatgaggccgagttttttgccgaagcgttccgtttgatgcattcaaccgaccacgcggaaagactgaaagtgcagaaaaacgccccaaagacattccagtttattaacgaccaaatcaagttcataatcaattcg

SEQ ID NO: 28 - DNA, 2427 bp, Artificial Sequence
  Synthetic coding region
atgaatattaagaaagaattcattaaagtcattagcatgagctgcctagtcaccgccatcaccctctctggcccagtgtttattccactggtacagggcgcaggcgggcatggcgacgtgggaatgcatgttaaggagaaggaaaaaaataaagatgaaaacaaacgcaaggacgaagaacggaacaagacccaggaggagcacctgaaagagatcatgaaacacattgtgaaaatcgaagttaaaggtgaagaggccgtgaagaaggaagccgcggagaaactgctggaaaaggtcccgtcggacgtacttgaaatgtacaaggcaattggtggcaaaatctacattgtggacggggacattaccaagcacataagcctggaagcactcagcgaggacaagaagaaaataaaggacatttacggaaaggacgctctgctccacgagcactatgtctacgcgaaggaggggtacgagcccgtgttggtgatacagagttccgaggactatgttgaaaatactgaaaaagccctcaacgtgtactatgagattggtaagatcttgtctagagacattctcagcaagattaaccagccctaccagaaattcctggatgtcctgaacacgattaagaatgcctcagacagcgatggacaggaccttctgtttaccaatcagcttaaagagcaccctaccgatttctccgtggaattccttgagcagaattcaaatgaggtgcaagaggtcttcgctaaggcctttgcctactatatcgagccccagcatcgagacgtgctacagttgtatgcaccagaagcctttaactacatggacaagttcaatgagcaagagatcaacttatcactggaggagctgaaggatcaacgcatgctgtctcggtatgaaaaatgggagaaaataaagcagcattaccagcattggagcgactccctgtctgaagagggtcgcggcctcctgaaaaagctgcagattcctatcgagcctaaaaaagatgatataattcactcactgtcccaggaagagaaggagctgcttaagcggatccagatagattccagtgacttcttaagcacggaagagaaggaatttctgaaaaaattgcagatcgatatccgtgattccctgtcagaggaggaaaaagaactgctgaaccggattcaggtggattcctccaatccactgtccgaaaaggagaaggagtttttaaagaaactgaagctcgatatccagccttacgatatcaatcaaaggttgcaggacacagggggattgattgactcgcccagtatcaacttggacgtgagaaagcagtacaaacgagacatacagaatatcgatgctctcctgcatcaaagcattgggtctacactttacaacaagatttatttgtacgaaaacatgaatatcaataatttgaccgccactttaggtgctgatctcgtggactccactgataacacaaagattaataggggcattttcaatgagttcaaaaagaatttcaagtattctattagctctaactatatgattgttgatatcaacgaacgaccagccctagacaacgaacggctaaagtggcggatccagctatcaccggacaccagagctggctacctcgaaaatgggaagctcattctccagaggaatatcggactggagataaaggacgttcaaataataaaacaaagcgagaaggagtacataaggatcgatgctaaggtcgtgcccaaaagtaagatcgatactaagattcaagaggctcaactgaatatcaatcaggagtggaataaggctctcgggctgcccaagtacacaaagctgatcacattcaatgttcataacagatacgcgtctaacatcgtcgagtccgcgtatctgatccttaatgaatggaagaacaacatccagtccgatctcatcaagaaagtcactaactacttagtagatgggaacggacgctttgtgtttacagatatcacactcccaaacatcgctgaacagtatacgcaccaagatgaaatctatgagcaggtgcacagtaagggcctgtacgtgcctgagagtagaagtatccttctgcacggccccagcaaaggcgtagagcttcgtaacgattcggagggattcatacatgagtttgggcacgcagtcgacgactatgccggttatcttctcgacaaaaaccagagcgacttggttaccaacagtaagaaatttatcgatatcttcaaagaagagggctctaacctaacatcatatggaaggactaacgaggcagaattctttgccgaagccttccgcctgatgcattcaaccgaccacgcagagagactgaaagtgcagaaaaacgcccctaagactttccagtttattaatgaccagatcaaatttatcatcaactct

SEQ ID NO: 29 - DNA, 26 bp, Artificial Sequence
  Synthetic PCR primer
gagcttgatatcgccaccatggatgc

SEQ ID NO: 30 - DNA, 29 bp, Artificial Sequence
  Synthetic PCR primer
ccaccaatatccgatgcatggacttccgc

SEQ ID NO: 31 - DNA, 28 bp, Artificial Sequence
  Synthetic PCR primer
cttgaaggatcctcaaccgatctcgtag

SEQ ID NO: 32 - DNA, 30 bp, Artificial Sequence
  Syntheric PCR Primer
ccatgcatcggatattggtggctccgtgtc

SEQ ID NO: 33 - DNA, 21 bp, Artificial Sequence
  Synthetic PCR primer
gtggacgaccaggaagtgatc

SEQ ID NO: 34 - DNA, 25 bp, Artificial Sequence
  Synthetic PCR primer
ggctatctgtccagtacagcttgaa

SEQ ID NO: 35 - DNA, 20 bp, Artificial Sequence
  Synthetic PCR primer
ccgtgctcgttattcagagt

SEQ ID NO: 36 - DNA, 20 bp, Artificial Sequence
  Synthetic PCR primer
ccttctcttctgtgctaagg

SEQ ID NO: 37 - DNA, 34 bp, Artificial Sequence
  Synthetic PCR primer
gaacctggatccctacaccaccttggcgtcgatg

SEQ ID NO: 38 - DNA, 35 bp, Artificial Sequence
  Synthetic PCR primer
gctaatggatcctcaaaatgccttggcgaacacct

SEQ ID NO: 39 - DNA, 876 bp, Artificial Sequence
  Synthetic coding region for Human/B. anthracis antigen fusion protein
  CDS (13)..(870)
gatatcgccaccatggatgcaatgaagagagggctctgctgtgtgctgctgctgtgtggagcagtcttcgtttcgcccagcgccggcgggcatggggacgttggcatgcatgtgaaagaaaaggagaaaaacaaggacgaaaacaagcgtaaagacgaagaacgtaataaaacacaggaggaacacttaaaggagatcatgaagcacatagtaaagattgaggtaaaaggcgaagaggctgtaaagaaggaggcagcagaaaaactgttggagaaggtgccttctgacgtcttagagatgtataaggccatcggcggtaagatctatatcgtggacggagacatcactaaacacatatctctcgaagctctctccgaggacaagaaaaagattaaagacatctacgggaaggatgccttattgcacgagcactacgtttacgcaaaggagggctatgagcccgtgctcgttattcagagtagtgaggactacgtcgagaataccgagaaagctctgaatgtgtattacgagatcggaaagattctgtcccgggacatcctgtccaaaatcaaccagccataccagaaattccttgatgttcttaacacaatcaaaaacgcgtcagatagcgacgggcaggatcttctgtttacaaatcaactcaaggaacaccccactgatttcagcgtggagttcctcgagcagaattctaacgaagtccaggaggtgttcgccaaggcatttgcgtactatatcgaaccccagcatcgcgatgtgctccagctgtacgccccggaggcatttaactacatggacaaattcaatgaacaggagattaatctgtctctggaggaactgaaagaccagtgaggatcc

SEQ ID NO: 40 - protein, 285 aa, Artificial Sequence
  Human/B. anthracis antigen fusion protein
MDAMKRGLCCVLLLCGAVFVSPSAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQ

SEQ ID NO: 41 - DNA, 35 bp, Artificial Sequence
  Synthetic PCR primer
ccatacggatcctcactggtctttcagttcctcca

SEQ ID NO: 42 - DNA, 24 bp, Artificial Sequence
  Synthetic PCR primer
ctggagacacctgtttatcgatcc

SEQ ID NO: 43 - DNA, 24 bp, Artificial Sequence
  Synthetic PCR primer
ggatcgataaacaggtgtctccag

SEQ ID NO: 44 - DNA, 26 bp, Artificial Sequence
  Synthetic PCR primer
gaagtactggtctgtttagatatggt

SEQ ID NO: 45 - DNA, 26 bp, Artificial Sequence
  Synthetic PCR primer
accatatctaaacagaccagtacttc

SEQ ID NO: 46 - DNA, 23 bp, Artificial Sequence
  Synthetic PCR primer
cgtcgaggactggctattgctaa

SEQ ID NO: 47 - DNA, 23 bp, Artificial Sequence
  Synthetic PCR primer
ttagcaatagccagtcctcgacg

SEQ ID NO: 48 - DNA, 22 bp, Artificial Sequence
  Synthetic PCR primer
gagggtctgctgtttgcccagg

SEQ ID NO: 49 - DNA, 22 bp, Artificial Sequence
  Synthetic PCR primer
cctgggcaaacagcagaccctc

SEQ ID NO: 50 - DNA, 23 bp, Artificial Sequence
  Synthetic PCR primer
cttcagaccactgtgacccagtg

SEQ ID NO: 51 - DNA, 23 bp, Artificial Sequence
  Synthetic PCR primer
cactgggtcacagtggtctgaag

SEQ ID NO: 52 - DNA, 21 bp, Artificial Sequence
  Synthetic PCR primer
gatcactgggctgcacggcgg

SEQ ID NO: 53 - DNA, 21 bp, Artificial Sequence
  Synthetic PCR primer
ccgccgtgcagcccagtgatc

SEQ ID NO: 54 - DNA, 22 bp, Artificial Sequence
  Synthetic PCR primer
tattggtggcctgcagctctgc

SEQ ID NO: 55 - DNA, 22 bp, Artificial Sequence
  Synthetic PCR primer
gcagagctgcaggccaccaata

SEQ ID NO: 56 - DNA, 24 bp, Artificial Sequence
  Synthetic PCR primer
cagtactgctctggataacttccc

SEQ ID NO: 57 - DNA, 24 bp, Artificial Sequence
  Synthetic PCR primer
gggaagttatccagagcagtactg

SEQ ID NO: 58 - DNA, 25 bp, Artificial Sequence
  Synthetic PCR primer
aagctggaaatctgcagcatatcat

SEQ ID NO: 59 - DNA, 25 bp, Artificial Sequence
  Synthetic PCR primer
atgatatgctgcagatttccagctt

SEQ ID NO: 60 - DNA, 23 bp, Artificial Sequence
  Synthetic PCR primer
ctcgcttggctggatgattgtgt

SEQ ID NO: 61 - DNA, 23 bp, Artificial Sequence
  Synthetic PCR primer
acacaatcatccagccaagcgag

SEQ ID NO: 62 - DNA, 24 bp, Artificial Sequence
  Synthetic PCR primer
tcctgtgttttctgacgttcttcg

SEQ ID NO: 63 - DNA, 24 bp, Artificial Sequence
  Synthetic PCR primer
cgaagaacgtcagaaaacacagga

SEQ ID NO: 64 - DNA, 25 bp, Artificial Sequence
  Synthetic PCR primer
tatctgacgcctgtttgattgtgtt

SEQ ID NO: 65 - DNA, 25 bp, Artificial Sequence
  Synthetic PCR primer
aacacaatcaaacaggcgtcagata

SEQ ID NO: 66 - DNA, 24 bp, Artificial Sequence
  Synthetic PCR primer
ccagagacagctgaatctcctgtt

SEQ ID NO: 67 - DNA, 24 bp, Artificial Sequence
  Synthetic PCR primer
aacaggagattcagctgtctctgg

SEQ ID NO: 68 - DNA, 25 bp, Artificial Sequence
  Synthetic PCR primer
agcggtgagctggttaatattcatg

SEQ ID NO: 69 - DNA, 25 bp, Artificial Sequence
  Synthetic PCR primer
catgaatattaaccagctcaccgct

SEQ ID NO: 70 - DNA, 23 bp, Artificial Sequence
  Synthetic PCR primer
cctcagaatcctgtcgaagctca

SEQ ID NO: 71 - DNA, 23 bp, Artificial Sequence
  Synthetic PCR primer
tgagcttcgacaggattctgagg

SEQ ID NO: 72 - DNA, 24 bp, Artificial Sequence
  Synthetic PCR primer
gatcagactgctgcttatccaaca

SEQ ID NO: 73 - DNA, 24 bp, Artificial Sequence
  Synthetic PCR primer
tgttggataagcagcagtctgatc

SEQ ID NO: 74 - DNA, 23 bp, Artificial Sequence
  Synthetic PCR primer
aggaagtcagctgactcccttcc

SEQ ID NO: 75 - DNA, 23 bp, Artificial Sequence
  Synthetic PCR primer
ggaagggagtcagctgacttcct

SEQ ID NO: 76 - DNA, 21 bp, Artificial Sequence
  Synthetic PCR primer
gcagatctggatcctcaagag